SEQUENCE LISTING (113 sequences)

SEQ ID NO: 1 - protein, 119 aa, Oryctolagus cuniculus
LSLEESGGRLVTPGTPLTLTCTVSGIDLSSYAMGWVRQAPGKGLEYIGIISSSGSAFYASWAKGRFTISKTSSTTVDLKMTSPTTEDTATYFCARHYAGYTTPYYFNFWGPGTLVTVSS

SEQ ID NO: 2 - protein, 110 aa, Oryctolagus cuniculus
AYDMTQTPASVEVTVGGTVTIKCQASENIYSNLAWYQQKPGQPPKVLIYAASDLASGVPSRFKGSGSGTEYTLTISGVECADAATYYCQQGFSSSNVDNAFGGGTEVVVK

SEQ ID NO: 3 - protein, 5 aa, Oryctolagus cuniculus
SYAMG

SEQ ID NO: 4 - protein, 16 aa, Oryctolagus cuniculus
IISSSGSAFYASWAKG

SEQ ID NO: 5 - protein, 13 aa, Oryctolagus cuniculus
HYAGYTTPYYFNF

SEQ ID NO: 6 - protein, 11 aa, Oryctolagus cuniculus
QASENIYSNLA

SEQ ID NO: 7 - protein, 7 aa, Oryctolagus cuniculus
AASDLAS

SEQ ID NO: 8 - protein, 12 aa, Oryctolagus cuniculus
QQGFSSSNVDNA

SEQ ID NO: 9 - protein, 126 aa, Oryctolagus cuniculus
QQLEQSGGGAEGGLVKPGGSLDLCCKASGFSLSSENWICWVRQAPGKGLEWIGCICGANGGSAYHANWVNGRFTLSRDIDQSTDCLHLNSLTASDTAMYYCARAAGVSNAYYFDLWGPGTLVTVSS

SEQ ID NO: 10 - protein, 110 aa, Oryctolagus cuniculus
DVVMTQTPSSVSAAVGGTVTIKCQASQYIYINLAWYQQKPGQRPKLLIYDASDLASGVPSRFKGSRSGTEFTLTISDLECADAATYYCQCTYGGSTYGNAFGGGTEVVVK

SEQ ID NO: 11 - protein, 6 aa, Oryctolagus cuniculus
SENWIC

SEQ ID NO: 12 - protein, 18 aa, Oryctolagus cuniculus
CICGANGGSAYHANWVNG

SEQ ID NO: 13 - protein, 12 aa, Oryctolagus cuniculus
AAGVSNAYYFDL

SEQ ID NO: 14 - protein, 11 aa, Oryctolagus cuniculus
QASQYIYINLA

SEQ ID NO: 15 - protein, 7 aa, Oryctolagus cuniculus
DASDLAS

SEQ ID NO: 16 - protein, 12 aa, Oryctolagus cuniculus
QCTYGGSTYGNA

SEQ ID NO: 17 - protein, 124 aa, Oryctolagus cuniculus
QEQLEESGGGLVKPEGSLTLTCKASGFDLSSAYYMCWVRQAPGKGLEWIGCIATGSGSTYYASWAKGRFTISKTSSTTVTLQATSLTAADTATLFCARAPYYSYDGYDYALNLWGPGTLVTVSS

SEQ ID NO: 18 - protein, 109 aa, Oryctolagus cuniculus
IKMTQTPSSVSAVVGGTVTINCQASEDIVTYLAWYQQKPGHSPNLLIYAASTLASGVPSRFKGSGSGTQFTLTISDVQCDDAATYYCQQTYNYADTDNAFGGGTEVVVK

SEQ ID NO: 19 - protein, 6 aa, Oryctolagus cuniculus
SAYYMC

SEQ ID NO: 20 - protein, 17 aa, Oryctolagus cuniculus
CIATGSGSTYYASWAKG

SEQ ID NO: 21 - protein, 15 aa, Oryctolagus cuniculus
APYYSYDGYDYALNL

SEQ ID NO: 22 - protein, 11 aa, Oryctolagus cuniculus
QASEDIVTYLA

SEQ ID NO: 23 - protein, 7 aa, Oryctolagus cuniculus
AASTLAS

SEQ ID NO: 24 - protein, 12 aa, Oryctolagus cuniculus
QQTYNYADTDNA

SEQ ID NO: 25 - protein, 128 aa, Oryctolagus cuniculus
QSLEESGGDLVKPGASLKLTCTASGFSFSSSYYMCWVRQAPGKGLEWVACFYAGFSGSTYYASWAKGRFTISKTSSTTVTLQMTSLTAADTATYFCAKGASTYGYTGYDYAGDYFILWGPGTLVTVSS

SEQ ID NO: 26 - protein, 112 aa, Oryctolagus cuniculus
ALVMTQTPSSVSAAVGGTVTINCQASQNIYSNLAWYQQKPGQRPKLLIYAASNLASGVSSRFKGSGSGTEYTLTISDLECDDAATYYCQSAYYSSSADSYNAFGGGTEVVVK

SEQ ID NO: 27 - protein, 6 aa, Oryctolagus cuniculus
SSYYMC

SEQ ID NO: 28 - protein, 18 aa, Oryctolagus cuniculus
CFYAGFSGSTYYASWAKG

SEQ ID NO: 29 - protein, 19 aa, Oryctolagus cuniculus
GASTYGYTGYDYAGDYFIL

SEQ ID NO: 30 - protein, 11 aa, Oryctolagus cuniculus
QASQNIYSNLA

SEQ ID NO: 31 - protein, 7 aa, Oryctolagus cuniculus
AASNLAS

SEQ ID NO: 32 - protein, 14 aa, Oryctolagus cuniculus
QSAYYSSSADSYNA

SEQ ID NO: 33 - protein, 113 aa, Oryctolagus cuniculus
QSLEESGGRLVTPGTPLTLTCTASGFSLSSYAMGWVRQAPGKGLEWIGIIIVSGNIYYASWAKGQFTISKTSTTVDLKITSPTTEDTATYFCARGDGYTYGLWGQGTLVTVSS

SEQ ID NO: 34 - protein, 111 aa, Oryctolagus cuniculus
DIVMTQTPASVEAAVGGTVTIKCQASQSVYSRYLAWFQQKPGQPPKLLIYSASTLASGVSSRFKGSGSGTEYTLTISGVQCDDAATYYCQGTFYIPDYIDGFGGGTEVVVK

SEQ ID NO: 35 - protein, 5 aa, Oryctolagus cuniculus
SYAMG

SEQ ID NO: 36 - protein, 16 aa, Oryctolagus cuniculus
IIIVSGNIYYASWAKG

SEQ ID NO: 37 - protein, 8 aa, Oryctolagus cuniculus
GDGYTYGL

SEQ ID NO: 38 - protein, 12 aa, Oryctolagus cuniculus
QASQSVYSRYLA

SEQ ID NO: 39 - protein, 7 aa, Oryctolagus cuniculus
SASTLAS

SEQ ID NO: 40 - protein, 12 aa, Oryctolagus cuniculus
QGTFYIPDYIDG

SEQ ID NO: 41 - protein, 128 aa, Oryctolagus cuniculus
QSLEESGGDLVKPGASLKLTCTASGFSFSSSYYMCWVRQAPGKGLEWIGCFYAGFSGGTYYASWAKGRFTISKTSSTTVTLQMTGLTAADTATYFCAKGANTYGYTGYDYAGDYFTLWGPGTLVTVSS

SEQ ID NO: 42 - protein, 111 aa, Oryctolagus cuniculus
ALVMTQTPSSVSAVVRGTVTINCQASQNIGSNLAWYQQKPGQRPKLLVYAASNLESGVPSRFKGSGSGTEFTLTISDLECDDAATYYCQSAYYSSVDSYNTFGGGTEVVVK

SEQ ID NO: 43 - protein, 6 aa, Oryctolagus cuniculus
SSYYMC

SEQ ID NO: 44 - protein, 18 aa, Oryctolagus cuniculus
CFYAGFSGGTYYASWAKG

SEQ ID NO: 45 - protein, 19 aa, Oryctolagus cuniculus
GANTYGYTGYDYAGDYFTL

SEQ ID NO: 46 - protein, 11 aa, Oryctolagus cuniculus
QASQNIGSNLA

SEQ ID NO: 47 - protein, 7 aa, Oryctolagus cuniculus
AASNLES

SEQ ID NO: 48 - protein, 13 aa, Oryctolagus cuniculus
QSAYYSSVDSYNT

SEQ ID NO: 49 - protein, 120 aa, Oryctolagus cuniculus
QSVEESGGRLVTPGTPLTLTCTASGFSLSDYAMTWVRQAPGKGLEYIGIISAGGYTYYASWATGRFTISKTSTTVDLKIPSPTAEDAATYFCARVGYADSSDIYKGFYLWGPGTLVTVSS

SEQ ID NO: 50 - protein, 120 aa, Oryctolagus cuniculus
QSVEESGGRLVTPGTPLTLTCTASGFSLSDYAMTWVRQAPGKGLEYIGIISAGGYTYYASWATGRFTISKTSTTVDLKIPSPTAEDAATYFCARVGYADSSDIYKGFYLWGPGTLATVSS

SEQ ID NO: 51 - protein, 116 aa, Oryctolagus cuniculus
QSLEESGGRLVTPGTPLTLTCTASGFFLNNYYMSWVRQAPGKGLEWIGLIYTSGSTLYANWAKGRFTISRTSTTVDLKITSPTTEDTATYFCARYDNNVDSWLDLWGQGTLVTVSS

SEQ ID NO: 52 - protein, 116 aa, Oryctolagus cuniculus
QSLEESGGHLVTPGTPPTLTCTASGFFLNNYYMSWVRQAPGKGLEWIGLIYTSGSTLYANWAKGRFTISRTSTTVDLKITSPTTEDTATYFCARYDNNVDSWLDLWGQGTLVTVSS

SEQ ID NO: 53 - protein, 118 aa, Oryctolagus cuniculus
QSLEESGGRLVTPGTPLTLTCTVSGIDLSSYAMAWVRQAPGEGLEWIGTISSSGHTYYPTWVKGRFTISKPSSTTVDLRMTSPTTEDTATYFCARGHYGYFGTVFALWGPGTLVTVSS

SEQ ID NO: 54 - protein, 118 aa, Oryctolagus cuniculus; identical to SEQ ID NO: 53

SEQ ID NO: 55 - protein, 123 aa, Oryctolagus cuniculus
QEQLVESGGGLVQPGGSLKLSCKASGFGFSSYGVSWVRQDPGKGLEWIGYIDPVLGSAAYATWVNGRFTISSHNAQNTLYLQLNSLTAADTATYFCARAGYPGYGYATNFNLWGPGTLVTVSS

SEQ ID NO: 56 - protein, 123 aa, Oryctolagus cuniculus
QEQLVESGGGLVQPGGSLKLSCKAPGFDFSSYGVSWVRQDPGKGLEWIGYIDPVLGSAAYATWVNGRFTISSHNAQNTLYLQLNSLTAADTATYFCARAGYPGYGYATNFNLWGPGTLVTVSS

SEQ ID NO: 57 - skipped

SEQ ID NO: 58 - protein, 118 aa, Oryctolagus cuniculus
QSVEESGGRLVTPGTTLTLTCTVSGFSLSLYYMSWVRQAPGKGLEWIGMTYTGGSAYYASWAKGRFTISQTSTTVDLKMTTLTVADTATYFCVRGYLADYYGDRDNLWGPGTLVTVSS

SEQ ID NO: 59 - protein, 109 aa, Oryctolagus cuniculus
QSVEESGGRLVTPGTPLTLTCTVSGFSLSSYWMSWVRQAPGEGLEWIGTIYTDGHTYYAKWAKGRFTISKTSTTVDLKMTSLTTADTATYFCARPWGVWGPGTLVTVSL

SEQ ID NO: 60 - protein, 116 aa, Oryctolagus cuniculus
QSVEESGGRLVTPGTPLTLTCTVSGFSLISYWMIWVRQAPGEGLEWIGFINTGGSAYYASWAKGRFTISRTSTTVDLKMTSLTTEDTATYFCARARYDSYITGDLWGPGTLVTVSS

SEQ ID NO: 61 - protein, 113 aa, Oryctolagus cuniculus
QSVEESGGRLVTPGTPLTLTCTVSGFSLSSANMGWVRQAPGEGLEYIGLIDTDGTTYYASWAKGRFTISRTSTTVDLKMTSLTTEDTATYFCARLRTGYGDLWGPGTLVAVSS

SEQ ID NO: 62 - protein, 120 aa, Oryctolagus cuniculus; identical to SEQ ID NO: 49

SEQ ID NO: 63 - protein, 114 aa, Oryctolagus cuniculus
QSVEESGGRLVTPGTPLTLTCTVSGFSLSSHAMSWVRQAPGKGLEWIGIIWTGGNTYYANWAKGRFTISKTSTTVDLKITSPIMEDTATYFCARYLNVEIGDVWGPGTLVTVSS

SEQ ID NO: 64 - protein, 113 aa, Oryctolagus cuniculus
QSVEESGGRLVAPGTPLTLTCAVSGFSLSSYDMSWVRQAPGKGLEYIGVIGTGSTGPYYTSWAKGRFTISKTSTTVDLKITSPTTEDTATYFCARGWVKLDLWGPGTPVTVSS

SEQ ID NO: 65 - protein, 117 aa, Oryctolagus cuniculus
QSVEESGGRLVTPGTPLTLACTVSGIDLSTNVMSWVRQAPGKGLEWIGIIFDSDITYYANWAKGRFTISKTSTTVDLKFTSPTTEDTATYFCARYDTYGYSYSIDLWGPGTLVTVSS

SEQ ID NO: 66 - protein, 116 aa, Oryctolagus cuniculus
QSLEESGGRLVTPGTPLTLTCTVSGIDLSDYAMGWVRQAPGKGLEYIAVISPIAYTYYAPWARGRFTISRASTTVDLKVTSPTTEDTATYFCARWAGTGWGYFNLWGPGTLVTVSS

SEQ ID NO: 67 - protein, 122 aa, Oryctolagus cuniculus
QELVESGGGLVQPGESLKLSCKASGFDFSSYGVSWVRQAPGKGLEWVGYIDPVLGSAAHASWVNGRFTISSHNAQNTLYLQLNSLTAADTATYFCARAGYAGYGYGTGFNLWGPGTLVTVSS

SEQ ID NO: 68 - protein, 126 aa, Oryctolagus cuniculus
QELVESGGGLVQAGESLKLSCKASGIDFSSYGISWVRQAPGKGLEWIAYIYPGNDITDYAHSVKGRFTISSDNAQNTVFLQMTSLTASDTATYFCARDAGYAGYGYPTGTYYFTLWGPGTLVTVSS

SEQ ID NO: 69 - protein, 126 aa, Oryctolagus cuniculus
QELVESGGGLVQAGESLKLSCKASGIDFSSYGISWVRQAPGKGLGWIAYIYPGNDITDYANSVKGRFTISSDNAQNTVFLQMTSLTASDTATYFCARDAGYAGDGYPTGTYYFTLWGPGTLVTVSS

SEQ ID NO: 70 - protein, 108 aa, Oryctolagus cuniculus
DVVMTQTPASVSEPVGGTVTIKCQASESISNYLAWYQQKPGQPPKLLIYLASTLASGVPSRFEGSRSGTEFTLTISDLECADAATYYCHCTDSIINYNFGGGTEVVVK

SEQ ID NO: 71 - protein, 111 aa, Oryctolagus cuniculus
AYDMTQTPASVEVAVGGTVTIKCQASQSISSSYLAWYQQKPGQPPKLLIYVTSTLASGVSSRFKGSGSGTQFTLTISGVECADAATYYCQQGYSVSNIDNVFGGGTEVVVK

SEQ ID NO: 72 - protein, 113 aa, Oryctolagus cuniculus
ADIVMTQTPSSVSAAVGGTVTINCQASESIQTYLAWYQQKPGQPPKLLIYDASDLASGVPSRFSGSGSGKQFTLTISGVQCDDAATYYCQNYYFSTNSNYGDVFGGGTEMVVK

SEQ ID NO: 73 - protein, 108 aa, Oryctolagus cuniculus
ALVMTQTPSSVSAAVGGTVTINCQASQNIYSNLAWYQQKPGQRPKLLIYGASNLESGVPSRFKGSGSGIEYTLTISDLECDDAATYYCQTNYYSHSAPFGGGTEVVVK

SEQ ID NO: 74 - protein, 111 aa, Oryctolagus cuniculus
IVMTQTPSSKSVPVGDTVTINCQASESVFSDDRLAWYQQKPGQPPKLLIYRASNLASGVPSRFSGSGSGTQFTLTINDVVCDDAATYYCAGYKGDSTDGVAFGGGTEVVVK

SEQ ID NO: 75 - protein, 112 aa, Oryctolagus cuniculus
QVLTQTPSPVSAAVGGTVTISCQSSESVYSNNRLSWFQQKPGQPPKLLIYQASKLASGVPSRFSGSGSGTQFTLTISGVQCDDAATYYCLGGYDCSSADCDTFGGGTEVVVR

SEQ ID NO: 76 - protein, 110 aa, Oryctolagus cuniculus
AVLTQTPSPVSAAVGGTVSISCQSSKSVSDNNWLSWYQQKPGQRPKLLIFYASTLASGVPSRFKGSGSGTQFTLTISDVQCDDAATYYCAGAYTNYNDNAFGGGTEVVVK

SEQ ID NO: 77 - protein, 111 aa, Oryctolagus cuniculus
QAVVTQTPSSVSAAVGGTVTISCQSSQSVYNNNFLSWYQQKPGQPPKLLIYRASNLETGVSSRFSGSGSGTQFTLTISGVQCDDAATYYCAGYYSSGWYYTFGGGTEVVVK

SEQ ID NO: 78 - protein, 108 aa, Oryctolagus cuniculus; identical to SEQ ID NO: 70

SEQ ID NO: 79 - protein, 113 aa, Oryctolagus cuniculus
QVLTQTPSSVSAAVGGTVTISCQSSQSVYKNNYLAWFQQKPGQPPKGLIYGASSLASGVSSRFKGSGSGTQFTLTISDVQCVDAATYYCQGCYYTAGSGDNYAFGGGTEVVVR

SEQ ID NO: 80 - protein, 111 aa, Oryctolagus cuniculus
QVLTQTPSPVSAAVGGTVTINCHSSQSVYNDNYLSWFQQKPGQPPKLLIYQASNLASGVPSRFKGSGSGTPFTLTIYDLECGDAATYYCAGGYNIGDSYSAFGGGTEVVVQ

SEQ ID NO: 81 - protein, 110 aa, Oryctolagus cuniculus
AYDMTQTPASAEVAVGGTVTINCQASEDIYNLLAWYQQKPGQRPKLLIYRASTLASGVSSRFKGSGSGTDYTLTISGVQCDDAATYYCQQGHSSRNVDNAFGGGTEVVVK

SEQ ID NO: 82 - protein, 110 aa, Oryctolagus cuniculus
AFELTQTPSSVSEPVGGTVTIKCQASESVSNYLAWYQQKPGQPPKLLIYGASTLESGVPSRFSGSGSGTEFTLTISDLECADAATYYCQEYDPSIDSGSVFGGGTELVVK

SEQ ID NO: 83 - protein, 109 aa, Oryctolagus cuniculus
QVLTQTASPVSAAVGGTVTINCQASQNIYSNLAWYQQKPGQRPKLLIYGASNLESGVPSRFRGSGSGTDYTLTISDLECDDAATYYCQSAYYSTSADNPFGGGTEVVVK

SEQ ID NO: 84 - protein, 111 aa, Oryctolagus cuniculus
ALVMTQTPSSVSAAVGGTVTINCQASQNIYNNLAWYQQKPGQRPKLLTYGASNLESGVPSRFKGSGSGTEYTLTISDLECDDAATYYCQSAYYSSSPDGAAFGGGTEVVVK

SEQ ID NO: 85 - protein, 111 aa, Oryctolagus cuniculus
ALVMTQTPSSVSAPVGGTVTINCQASQSIYNNLAWYQQKPGQRPKLLIYGASNLESGVPSRFEGSGSGTEYTLTISDLECDDAATYYCQSAYYSSSPDGAAFGGGTEVVVK

SEQ ID NO: 86 - protein, 121 aa, Artificial Sequence
  Made in Lab - humanized anti-VISTA antibody sequence
EVQLVESGGGLVQPGGSLRLSCAASGIDLSSYAMGWVRQAPGKGLEWVGIISSSGSAFYASWAKGRFTISRDNSKNTLYLQMNSLRAEDTAVYYCARHYAGYTTPYYFNFWGQGTLVTVSS

SEQ ID NO: 87 - protein, 110 aa, Artificial Sequence
  Made in Lab - humanized anti-VISTA antibody sequence
AIQMTQSPSSLSASVGDRVTITCQASENIYSNLAWYQQKPGKAPKVLIYAASDLASGVPSRFSGSGSGTDFTLTISSLQPEDFATYYCQQGFSSSNVDNAFGGGTKVEIK

SEQ ID NO: 88 - protein, 123 aa, Artificial Sequence
  Made in Lab - humanized anti-VISTA antibody sequence
EVQLVESGGGLVQPGGSLRLSCAASGFSLSSENWICWVRQAPGKGLEWVGCICGANGGSAYHANWVNGRFTISRDNSKNTLYLQMNSLRAEDTAVYYCARAAGVSNAYYFDLWGQGTLVTVSS

SEQ ID NO: 89 - protein, 110 aa, Artificial Sequence
  Made in Lab - humanized anti-VISTA antibody sequence
DIQMTQSPSSVSASVGDRVTITCQASQYIYINLAWYQQKPGKAPKLLIYDASDLASGVPSRFSGSGSGTDFTLTISSLQPEDFATYYCQCTYGGSTYGNAFGGGTKVEIK

SEQ ID NO: 90 - protein, 125 aa, Artificial Sequence
  Made in Lab - humanized anti-VISTA antibody sequence
EVQLVESGGGLVQPGGSLRLSCAASGFDLSSAYYMCWVRQAPGKGLEWVGCIATGSGSTYYASWAKGRFTISRDNSKNTLYLQMNSLRAEDTAVYYCARAPYYSYDGYDYALNLWGQGTLVTVSS

SEQ ID NO: 91 - protein, 110 aa, Artificial Sequence
  Made in Lab - humanized anti-VISTA antibody sequence
DIQMTQSPSSVSASVGDRVTITCQASEDIVTYLAWYQQKPGKAPKLLIYAASTLASGVPSRFSGSGSGTDFTLTISSLQPEDFATYYCQQTYNYADTDNAFGGGTKVEIK

SEQ ID NO: 92 - protein, 130 aa, Artificial Sequence
  Made in Lab - humanized anti-VISTA antibody sequence
EVQLLESGGGLVQPGGSLRLSCAASGFSFSSSYYMCWVRQAPGKGLEWVACFYAGFSGSTYYASWAKGRFTISRDNSKNTLYLQMNSLRAEDTAVYYCAKGASTYGYTGYDYAGDYFILWQPGTLVTVSS

SEQ ID NO: 93 - protein, 112 aa, Artificial Sequence
  Made in Lab - humanized anti-VISTA antibody sequence
DIQMTQSPSSVSASVGDRVTITCQASQNIYSNLAWYQQKPGKAPKLLIYAASNLASGVPSRFSGSGSGTDFTLTISSLQPEDFATYYCQSAYYSSSADSYNAFGGGTKVEIK

SEQ ID NO: 94 - protein, 116 aa, Artificial Sequence
  Made in Lab - humanized anti-VISTA antibody sequence
EVQLVESGGGLVQPGGSLRLSCAASGFSLSSYAMGWVRQAPGKGLEWVGIIIVSGNIYYASWAKGRFTISRDNSKNTLYLQMNSLRAEDTAVYYCARGDGYTYGLWGQGTLVTVSS

SEQ ID NO: 95 - protein, 111 aa, Artificial Sequence
  Made in Lab - humanized anti-VISTA antibody sequence
DIQMTQSPSTLSASVGDRVTITCQASQSVYSRYLAWYQQKPGKAPKLLIYSASTLASGVPSRFSGSGSGTEFTLTISSLQPDDFATYYCQGTFYIPDYIDGFGGGTKVEIK

SEQ ID NO: 96 - protein, 116 aa, Artificial Sequence; identical to SEQ ID NO: 94
  Made in Lab - humanized anti-VISTA antibody sequence

SEQ ID NO: 97 - protein, 111 aa, Artificial Sequence
  Made in Lab - humanized anti-VISTA antibody sequence
EIVMTQSPATLSLSPGERATLSCQASQSVYSRYLAWYQQKPGQAPRLLIYSASTLASGIPARFSGSGSGTDFTLTISSLQPEDFAVYYCQGTFYIPDYIDGFGGGTKVEIK

SEQ ID NO: 98 - protein, 130 aa, Artificial Sequence
  Made in Lab - humanized anti-VISTA antibody sequence
EVQLLESGGGLVQPGGSLRLSCAASGFSFSSSYYMCWVRQAPGKGLEWVGCFYAGFSGGTYYASWAKGRFTISRDNSKNTLYLQMNSLRAEDTAVYYCAKGANTYGYTGYDYAGDYFTLWGQGTLVTVSS

SEQ ID NO: 99 - protein, 111 aa, Artificial Sequence
  Made in Lab - humanized anti-VISTA antibody sequence
DIQMTQSPSTLSASVGDRVTITCQASQNIGSNLAWYQQKPGKAPKLLIYAASNLESGVPSRFSGSGSGTEFTLTISSLQPDDFATYYCQSAYYSSVDSYNTFGGGTKVEIK

SEQ ID NO: 100 - protein, 451 aa, Artificial Sequence
  Made in Lab - humanized anti-VISTA antibody sequence
EVQLVESGGGLVQPGGSLRLSCAASGIDLSSYAMGWVRQAPGKGLEWVGIISSSGSAFYASWAKGRFTISRDNSKNTLYLQMNSLRAEDTAVYYCARHYAGYTTPYYFNFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 217 aa, Artificial Sequence
  Made in Lab - humanized anti-VISTA antibody sequence
AIQMTQSPSSLSASVGDRVTITCQASENIYSNLAWYQQKPGKAPKVLIYAASDLASGVPSRFSGSGSGTDFTLTISSLQPEDFATYYCQQGFSSSNVDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 453 aa, Artificial Sequence
  Made in Lab - humanized anti-VISTA antibody sequence
EVQLVESGGGLVQPGGSLRLSCAASGFSLSSENWICWVRQAPGKGLEWVGCICGANGGSAYHANWVNGRFTISRDNSKNTLYLQMNSLRAEDTAVYYCARAAGVSNAYYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 217 aa, Artificial Sequence
  Made in Lab - humanized anti-VISTA antibody sequence
DIQMTQSPSSVSASVGDRVTITCQASQYIYINLAWYQQKPGKAPKLLIYDASDLASGVPSRFSGSGSGTDFTLTISSLQPEDFATYYCQCTYGGSTYGNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 455 aa, Artificial Sequence
  Made in Lab - humanized anti-VISTA antibody sequence
EVQLVESGGGLVQPGGSLRLSCAASGFDLSSAYYMCWVRQAPGKGLEWVGCIATGSGSTYYASWAKGRFTISRDNSKNTLYLQMNSLRAEDTAVYYCARAPYYSYDGYDYALNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 217 aa, Artificial Sequence
  Made in Lab - humanized anti-VISTA antibody sequence
DIQMTQSPSSVSASVGDRVTITCQASEDIVTYLAWYQQKPGKAPKLLIYAASTLASGVPSRFSGSGSGTDFTLTISSLQPEDFATYYCQQTYNYADTDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 460 aa, Artificial Sequence
  Made in Lab - humanized anti-VISTA antibody sequence
EVQLLESGGGLVQPGGSLRLSCAASGFSFSSSYYMCWVRQAPGKGLEWVACFYAGFSGSTYYASWAKGRFTISRDNSKNTLYLQMNSLRAEDTAVYYCAKGASTYGYTGYDYAGDYFILWQPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 219 aa, Artificial Sequence
  Made in Lab - humanized anti-VISTA antibody sequence
DIQMTQSPSSVSASVGDRVTITCQASQNIYSNLAWYQQKPGKAPKLLIYAASNLASGVPSRFSGSGSGTDFTLTISSLQPEDFATYYCQSAYYSSSADSYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 446 aa, Artificial Sequence
  Made in Lab - humanized anti-VISTA antibody sequence
EVQLVESGGGLVQPGGSLRLSCAASGFSLSSYAMGWVRQAPGKGLEWVGIIIVSGNIYYASWAKGRFTISRDNSKNTLYLQMNSLRAEDTAVYYCARGDGYTYGLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 218 aa, Artificial Sequence
  Made in Lab - humanized anti-VISTA antibody sequence
DIQMTQSPSTLSASVGDRVTITCQASQSVYSRYLAWYQQKPGKAPKLLIYSASTLASGVPSRFSGSGSGTEFTLTISSLQPDDFATYYCQGTFYIPDYIDGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 446 aa, Artificial Sequence; identical to SEQ ID NO: 108
  Made in Lab - humanized anti-VISTA antibody sequence

SEQ ID NO: 111 - protein, 218 aa, Artificial Sequence
  Made in Lab - humanized anti-VISTA antibody sequence
EIVMTQSPATLSLSPGERATLSCQASQSVYSRYLAWYQQKPGQAPRLLIYSASTLASGIPARFSGSGSGTDFTLTISSLQPEDFAVYYCQGTFYIPDYIDGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 460 aa, Artificial Sequence
  Made in Lab - humanized anti-VISTA antibody sequence
EVQLLESGGGLVQPGGSLRLSCAASGFSFSSSYYMCWVRQAPGKGLEWVGCFYAGFSGGTYYASWAKGRFTISRDNSKNTLYLQMNSLRAEDTAVYYCAKGANTYGYTGYDYAGDYFTLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 218 aa, Artificial Sequence
  Made in Lab - humanized anti-VISTA antibody sequence
DIQMTQSPSTLSASVGDRVTITCQASQNIGSNLAWYQQKPGKAPKLLIYAASNLESGVPSRFSGSGSGTEFTLTISSLQPDDFATYYCQSAYYSSVDSYNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2021/0139589 A1
(43) Pub. Date: May 13, 2021

(54) ANTI-VISTA ANTIBODIES AND METHODS OF USE

(71) Applicant: APEXIGEN, INC, San Carlos, CA (US)

(72) Inventors: Erin L. Filbert, San Carlos, CA (US); Christine TAN, San Carlos, CA (US); Pia BJÖRCK, San Carlos, CA (US); Xiaodong YANG, San Carlos, CA (US)

(21) Appl. No.: 16/623,601

(22) PCT Filed: Jun. 22, 2018
(86) PCT No.: PCT/US2018/039036

§ 371 Date: Dec. 17, 2019

Related U.S. Application Data
(60) Provisional application No. 62/523,649, filed on Jun. 22, 2017.

(51) Int. Cl. C07K 16/28; A61K 35/17; C07K 14/54

(52) U.S. Cl. CPC C07K 16/2827; A61K 35/17; C07K 14/54; C07K 2317/565; C07K 2317/732; C07K 2317/92; C07K 2317/734

(57) ABSTRACT

Provided are anti-VISTA monoclonal antibodies and related compositions, which may be used in any of a variety of therapeutic methods for the treatment of a variety of inflammatory, oncological, and infectious diseases.

### CROSS-REFERENCE TO RELATED APPLICATIONS

This application claims priority under 35 U.S.C. § 119(e) to U.S. Application No. 62/523,649, filed Jun. 22, 2017, which is incorporated by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is APEX-019_01 WO_ST25.txt. The text file is 114 KB, was created on Jun. 22, 2018 and is being submitted electronically via EFS-Web.

### BACKGROUND

### Technical Field

The present disclosure relates generally to anti-VISTA antibodies, compositions and methods of using and manufacturing same. Such antibodies are useful, for example, in methods for treating a variety of inflammatory, oncological, and infectious diseases.

### Description of the Related Art

Negative checkpoint regulators, or inhibitory immune checkpoints, play an important role in tempering immune responses; however, negative checkpoint regulators, such as programmed cell death protein 1 (PD-1), programmed death-ligand 1 (PD-L1), and cytotoxic T lymphocyte associated protein 4 (CTLA-4), can provide an environment in which tumors can thrive. Immune checkpoint inhibitors, including blocking antibodies specific for PD-1, PD-L1, and CTLA-4, have been identified as promising therapeutic agents for cancer therapies in an effort to reverse immunosuppression induced by these negative checkpoint regulators.

More recently, V-domain Ig suppressor of T cell activation (VISTA), which is also known as PD-1H, Dies1, platelet receptor Gi24, SISP1, C10orf54, and B7-H5, has become a promising target for immunotherapy (Wang et al., J Exp Med 2011 208(3):577-592). Anti-VISTA antibodies have been described for use in antagonistic roles to block immunosuppression and enhance T cell responses (U.S. Pat. No. 8,426,563; WO 2015/097536; and WO 2016/207717) and agonistic roles to enhance immunosuppression and treat autoimmune diseases, transplant rejections, and inflammatory diseases (US 2016/0096891 and Flies et al., J Immunology 2011 187:1537-1541). Combination treatments utilizing a VISTA antagonist in combination with PD-1 and PD-L1 inhibitors have also been suggested for modulating T cell responses (US 2016/0083472; Lines et al., Cancer Immunol Res 2014 2(6):510-517; and Liu et al., PNAS 2015 112(21):6682-6687; Nowak et al. Immunological Reviews 2017 276:66-79). To date, only a single anti-VISTA antibody has entered clinical trials, namely JNJ-61610588 (NCT02671955).

Accordingly, there remains a need in the art for therapeutic compositions and related methods of treating cancer that activate T cells and antigen presenting cells (APCs) and enhance immune surveillance to provide improved anti-cancer properties.

### BRIEF SUMMARY

The present disclosure relates to antibodies and antigen-binding fragments thereof the specifically bind to V-domain Ig suppressor of T cell activation (VISTA) and methods of use thereof. One aspect of the disclosure provides an isolated antibody, or an antigen-binding fragment thereof, that binds to VISTA, comprising (i) a heavy chain variable region comprising a VHCDR1, a VHCDR2, and a VHCDR3 of SEQ ID NOs: 3-5, 11-13, 19-21, 2-29, 35-37, or 43-45; and (ii) a light chain variable region comprising a VLCDR1, a VLCDR2, and a VLCDR3 of SEQ ID NOs: 6-8, 14-16, 22-24, 30-32, 38-40, or 46-48, respectively; or a variant of said antibody, or an antigen-binding fragment thereof, comprising heavy and light chain variable regions identical to the heavy and light chain variable regions of (i) and (ii) except for up to 8 amino acid substitutions in said CDR regions.

In one embodiment, the isolated antibody, or antigen-binding fragment thereof, the heavy chain variable region comprises the amino acid sequence set forth in SEQ ID NO:1, 9, 17, 25, 33, or 41, respectively. In one embodiment, the isolated antibody, or antigen-binding fragment thereof, the light chain variable region comprises the amino acid sequence set forth in SEQ ID NO:2, 10, 18, 26, 34, or 42, respectively.

Another aspect of the disclosure provides an isolated antibody, or an antigen-binding fragment thereof, that binds to human VISTA, comprising a heavy chain variable region comprising the amino acid sequence set forth in SEQ ID NO:1, 9, 17, 25, 33, or 41. In one embodiment, the light chain variable region comprises an amino acid sequence having at least 90% identity to the amino acid sequence set forth in SEQ ID NO: 2, 10, 18, 26, 34, or 42, respectively. In another embodiment, the light chain variable region which comprises the amino acid sequence set forth in SEQ ID NO: 2, 10, 18, 26, 34, or 42, respectively.

One aspect of the disclosure provides an isolated antibody, or an antigen-binding fragment thereof, that binds to human VISTA, comprising a light chain variable region comprising the amino acid sequence set forth in SEQ ID NO: 2, 10, 18, 26, 34, or 42, respectively. In one embodiment, the heavy chain variable region which comprises an amino acid sequence having at least 90% identity to the amino acid sequence set forth in SEQ ID NO: 1, 9, 17, 25, 33, or 41, respectively.

In one aspect of the disclosure, the antibody is humanized. In one embodiment, the VH region comprises SEQ ID NO:86, 88, 90, 92, 94, 96, or 98 and the VL region comprises SEQ ID NO:87, 89, 91, 93, 95, 97, or 99, respectively.

In one embodiment, the antibody is selected from the group consisting of a single chain antibody, a scFv, a univalent antibody lacking a hinge region, and a minibody. In another embodiment, the antibody is a Fab or a Fab′ fragment. In one embodiment, the antibody is a F(ab′)2 fragment. In one embodiment, the antibody is a whole antibody.

In certain embodiments, the antibody comprises a human IgG constant domain. In one embodiment, the IgG constant domain comprises an IgG1 CH1 domain. In another embodiment, the IgG constant domain comprises an IgG1 Fc region.

In some embodiments, the antibody comprises a modified Fc region, for example, wherein the modified Fc region has altered (e.g., enhanced, decreased) binding affinity for a specific FcγR, increased serum half-life, and/or altered (e.g., enhanced, decreased) effector function selected from one or more or of complement-dependent cytotoxicity (CDC), antibody-dependent cell-mediated cytotoxicity (ADCC), and antibody-dependent cell-mediated phagocytosis (ADCP).

In one embodiment, the isolated antibody, or antigen-binding fragment thereof, of claim 1 that binds VISTA with a KD of 2.2 nM or lower. In one embodiment, the isolated antibody, or antigen-binding fragment thereof: (a) increases T cell activation; (b) increases T cell proliferation; (c) increases MHC II expression; (d) activates natural killer (NK) cells; (e) activates monocytes/macrophages; (0 increases cytokine production, for example, wherein the cytokine is selected from one or more of IFN-gamma, IL-6, IL-1ra, IL-1a, IL-8, MIP-1a, MIP-1 b IP-10, TNF-alpha and MCP-1; or (g) a combination of any one or more of (a)-(f).

In certain embodiments, the isolated antibody, or antigen-binding fragment thereof, is a VISTA antagonist. In certain other embodiments, the isolated antibody, or antigen-binding fragment thereof, is a VISTA agonist.

One aspect of the disclosure provides an isolated antibody, or an antigen-binding fragment thereof, that binds to VISTA, comprising (i) a heavy chain variable region comprising the VHCDR1, VHCDR2, and VHCDR3 of any one of the VH regions shown in FIG. 1; and (ii) a light chain variable region comprising the VLCDR1, the VLCDR2, and the VLCDR3 region of the corresponding VL region of any one of the antibodies shown in FIG. 1; or a variant of said antibody, or an antigen-binding fragment thereof, comprising heavy and light chain variable regions identical to the heavy and light chain variable regions of (i) and (ii) except for up to 8 amino acid substitutions in said CDR regions.

Another aspect of the disclosure provides an isolated antibody, or an antigen-binding fragment thereof, that binds to VISTA, comprising a heavy chain variable region comprising any one of the VH regions shown in FIG. 1. In one embodiment, light chain variable region comprises an amino acid sequence having at least 90% identity to the corresponding VL region as shown in FIG. 1. In another embodiment, the VL comprises a corresponding light chain variable region as shown in FIG. 1.

One aspect of the disclosure provides an isolated antibody, or an antigen-binding fragment thereof, that binds to VISTA, comprising a light chain variable region comprising any one of the VL regions shown in FIG. 1. In one embodiment, the heavy chain variable region comprises an amino acid sequence having at least 90% identity to the corresponding VH region as shown in FIG. 1.

Another aspect of the disclosure provides an isolated polynucleotide encoding the isolated antibody, or antigen-binding fragment thereof. One aspect of the disclosure provides an expression vector comprising the isolated polynucleotide. A related aspect of the disclosure provides an isolated host cell comprising the vector.

One aspect of the disclosure provides a composition comprising a physiologically acceptable carrier and a therapeutically effective amount of the isolated antibody or antigen-binding fragment thereof. In one embodiment, a method for treating a patient having a cancer associated with aberrant VISTA expression, comprises administering to the patient the composition, thereby treating the cancer associated with aberrant VISTA expression. In another embodiment, a method for treating a patient having a cancer associated with VISTA-mediated immune suppression, comprises administering to the patient the composition, thereby treating the cancer associated with VISTA-mediated immune suppression.

Certain embodiments include administering to the patient at least one cancer immunotherapy agent. In some embodiments, the at least one cancer immunotherapy agent is selected from one or more of an immune checkpoint modulatory agent, a cancer vaccine, an oncolytic virus, a cytokine, and a cell-based immunotherapies.

In some embodiments, the immune checkpoint modulatory agent is a polypeptide, optionally an antibody or antigen-binding fragment thereof or a ligand, or a small molecule. In some embodiments, the immune checkpoint modulatory agent comprises
(a) an antagonist of a inhibitory immune checkpoint molecule; or
(b) an agonist of a stimulatory immune checkpoint molecule, optionally, wherein the immune checkpoint modulatory agent specifically binds to the immune checkpoint molecule.

In some embodiments, the inhibitory immune checkpoint molecule is selected from one or more of Programmed Death-Ligand 1 (PD-L1), Programmed Death 1 (PD-1), Programmed Death-Ligand 2 (PD-L2), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), B and T Lymphocyte Attenuator (BTLA), CD160, Herpes Virus Entry Mediator (HVEM), and T-cell immunoreceptor with Ig and ITIM domains (TIGIT).

In some embodiments, the antagonist is a PD-L1 and/or PD-L2 antagonist optionally selected from one or more of an antibody or antigen-binding fragment or small molecule that specifically binds thereto, atezolizumab (MPDL3280A), avelumab (MSB0010718C), and durvalumab (MED14736), optionally wherein the cancer is selected from one or more of colorectal cancer, melanoma, breast cancer, non-small-cell lung carcinoma, bladder cancer, and renal cell carcinoma;

the antagonist is a PD-1 antagonist optionally selected from one or more of an antibody or antigen-binding fragment or small molecule that specifically binds thereto, nivolumab, pembrolizumab, MK-3475, AMP-224, AMP-514PDR001, and pidilizumab, optionally wherein the PD-1 antagonist is nivolumab and the cancer is optionally selected from one or more of Hodgkin's lymphoma, melanoma, non-small cell lung cancer, hepatocellular carcinoma, renal cell carcinoma, and ovarian cancer;

the PD-1 antagonist is pembrolizumab and the cancer is optionally selected from one or more of melanoma, non-small cell lung cancer, small cell lung cancer, head and neck cancer, and urothelial cancer;

the antagonist is a CTLA-4 antagonist optionally selected from one or more of an antibody or antigen-binding fragment or small molecule that specifically binds thereto, ipilimumab, tremelimumab, optionally wherein the cancer is selected from one or more of melanoma, prostate cancer, lung cancer, and bladder cancer;

the antagonist is an IDO antagonist optionally selected from one or more of an antibody or antigen-binding fragment or small molecule that specifically binds thereto, indoximod (NLG-8189), 1-methyl-tryptophan (1MT), β-Carboline (norharmane; 9H-pyrido[3,4-b]indole), rosmarinic acid, and epacadostat, and wherein the cancer is optionally selected from one or more of metastatic breast cancer and brain cancer optionally glioblastoma multiforme, glioma, gliosarcoma or malignant brain tumor;

the antagonist is a TDO antagonist optionally selected from one or more of an antibody or antigen-binding fragment or small molecule that specifically binds thereto, 680C91, and LM10;

the antagonist is a TIM-3 antagonist optionally selected from one or more of an antibody or antigen-binding fragment or small molecule that specifically binds thereto;

the antagonist is a LAG-3 antagonist optionally selected from one or more of an antibody or antigen-binding fragment or small molecule that specifically binds thereto, and BMS-986016;

the antagonist is a BTLA, CD160, and/or HVEM antagonist optionally selected from one or more of an antibody or antigen-binding fragment or small molecule that specifically binds thereto; and/or

the antagonist is a TIGIT antagonist optionally selected from one or more of an antibody or antigen-binding fragment or small molecule that specifically binds thereto.

In some embodiments, the stimulatory immune checkpoint molecule is selected from one or more of CD40, OX40, Glucocorticoid-Induced TNFR Family Related Gene (GITR), CD137 (4-1BB), CD27, CD28, CD226, and Herpes Virus Entry Mediator (HVEM).

In some embodiments, the agonist is a CD40 agonist optionally selected from one or more of an antibody or antigen-binding fragment or small molecule or ligand that specifically binds thereto, APX005, APX005M, CP-870,893, dacetuzumab, Chi Lob 7/4, ADC-1013, and rhCD40L, and wherein the cancer is optionally selected from one or more of melanoma, pancreatic carcinoma, mesothelioma, and hematological cancers optionally lymphoma such as Non-Hodgkin's lymphoma;

the agonist is an OX40 agonist optionally selected from one or more of an antibody or antigen-binding fragment or small molecule or ligand that specifically binds thereto, OX86, Fc-OX40L, and GSK3174998;

the agonist is a GITR agonist optionally selected from one or more of an antibody or antigen-binding fragment or small molecule or ligand that specifically binds thereto, INCAGN01876, DTA-1, and MED11873;

the agonist is a CD137 agonist optionally selected from one or more of an antibody or antigen-binding fragment or small molecule or ligand that specifically binds thereto, utomilumab, and 4-1BB ligand;

the agonist is a CD27 agonist optionally selected from one or more of an antibody or antigen-binding fragment or small molecule or ligand that specifically binds thereto, varlilumab, and CDX-1127 (1F5);

the agonist is a CD28 agonist optionally selected from one or more of an antibody or antigen-binding fragment or small molecule or ligand that specifically binds thereto, and TAB08; and/or

the agonist is an HVEM agonist optionally selected from one or more of an antibody or antigen-binding fragment or small molecule or ligand that specifically binds thereto.

In some embodiments, the cancer vaccine is selected from one or more of Oncophage, a human papillomavirus HPV vaccine optionally Gardasil or Cervarix, a hepatitis B vaccine optionally Engerix-B, Recombivax HB, or Twinrix, and sipuleucel-T (Provenge), or comprises a cancer antigen selected from one or more of human Her2/neu, Her1/EGF receptor (EGFR), Her3, A33 antigen, B7H3, CD5, CD19, CD20, CD22, CD23 (IgE Receptor), MAGE-3, C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin, optionally wherein the subject has or is at risk for having a cancer that comprises the corresponding cancer antigen.

In some embodiments, the oncolytic virus selected from one or more of talimogene laherparepvec (T-VEC), coxsackievirus A21 (CAVATAK™), Oncorine (H101), pelareorep (REOLYSIN®), Seneca Valley virus (NTX-010), Senecavirus SVV-001, ColoAd1, SEPREHVIR (HSV-1716), CGTG-102 (Ad5/3-D24-GMCSF), GL-ONC1, MV-NIS, and DNX-2401.

In some embodiments, the cytokine selected from one or more of interferon (IFN)-α, IL-2, IL-12, IL-7, IL-21, and Granulocyte-macrophage colony-stimulating factor (GM-CSF).

In some embodiments, the cell-based immunotherapy agent comprises cancer antigen-specific T-cells, optionally ex vivo-derived T-cells.

In some embodiments, the cancer antigen-specific T-cells are selected from one or more of chimeric antigen receptor (CAR)-modified T-cells, and T-cell Receptor (TCR)-modified T-cells, tumor infiltrating lymphocytes (TILs), and peptide-induced T-cells.

In some embodiments, the anti-VISTA antibody, or antigen-binding fragment thereof, and the at least one cancer immunotherapy agent are administered separately, as separate compositions. In some embodiments, the anti-VISTA antibody, or antigen-binding fragment thereof, and the at least one cancer immunotherapy agent are administered together as part of the same composition.

Also included are methods for treating a patient having an infectious disease, comprising administering to the patient a composition comprising an anti-VISTA antibody, or antigen-binding fragment thereof, as described herein, thereby treating the infectious disease. In some embodiments, the infectious disease is a viral, bacterial, fungal optionally yeast, or protozoal infection.

Also included are methods of treating cancer in a patient in need thereof, comprising: (a) incubating ex vivo-derived, autologous immune cells obtained from the patient with an anti-VISTA antibody, or antigen-binding fragment thereof, described herein; and (b) administering the autologous immune cells to the patient. In some embodiments, the autologous immune cells comprise lymphocytes, natural killer (NK) cells, macrophages, and/or dendritic cells. In some embodiments, the lymphocytes comprise T-cells, optionally cytotoxic T-lymphocytes (CTLs). In some embodiments, the T-cells comprise cancer antigen-specific T-cells. In some embodiments, the cancer antigen-specific T-cells are selected from one or more of chimeric antigen receptor (CAR)-modified T-cells, T-cell Receptor (TCR)-modified T-cells, tumor infiltrating lymphocytes (TILs), and peptide-induced T-cells.

### BRIEF DESCRIPTION OF THE SEVERAL VIEWS OF THE DRAWINGS

FIGS. 1A-1F show the amino acid sequence alignments of rabbit anti-VISTA VH regions (FIGS. 1A-1C) and VL regions (FIGS. 1D-1F). Heavy chain and light chain CDRs 1-3 are underlined.

FIG. 2 is a line graph that shows the epitope binning of rabbit-human chimeric anti-VISTA antibodies in comparison to the human anti-VISTA antibody VSTB112.

FIG. 3 is a line graph showing activation of human monocytes by chimeric anti-VISTA antibodies in comparison to the human anti-VISTA antibody VSTB112.

FIG. 4 is a line graph showing the binding of humanized anti-VISTA antibodies to soluble VISTA as measured by ELISA.

FIG. 5 is a line graph showing the binding of humanized anti-VISTA antibodies to VISTA on the surface of transfected HEK293 cells as measured by flow cytometry.

FIGS. 6A-6G are a series of line graphs showing the enhancement of Staphylococcus enterotoxin B (SEB) stimulation of T cells by humanized anti-VISTA antibodies.

FIGS. 7A-7F are a series of line graphs showing the enhancement of CD4 T cell proliferation in a mixed lymphocyte reaction by humanized anti-VISTA antibodies.

FIGS. 8A-8C show the ability of anti-VISTA monoclonal antibodies to activate human NK cells.

FIGS. 9A-9I show the ability of humanized anti-VISTA antibodies to induce cytokine release in a whole blood assay.

### BRIEF DESCRIPTION OF THE SEQUENCES

The amino acid sequence identifiers of the VH regions, VL regions and CDRs of rabbit anti-VISTA antibody clones 2D12, 3A5, 14D8, 14F1, 29G7, and 41A11 are provided in Table 1 below.

TABLE 1
Rabbit Anti-VISTA Antibody SEQ ID NOs

| Clone | VH | VHCDR1 | VHCDR2 | VHCDR3 | VL | VLCDR1 | VLCDR2 | VLCDR3 |
|---|---|---|---|---|---|---|---|---|
| 2D12 | 1 | 3 | 4 | 5 | 2 | 6 | 7 | 8 |
| 3A5 | 9 | 11 | 12 | 13 | 10 | 14 | 15 | 16 |
| 14D8 | 17 | 19 | 20 | 21 | 18 | 22 | 23 | 24 |
| 14F1 | 25 | 27 | 28 | 29 | 26 | 30 | 31 | 32 |
| 29G7 | 33 | 35 | 36 | 37 | 34 | 38 | 39 | 40 |
| 41A11 | 41 | 43 | 44 | 45 | 42 | 46 | 47 | 48 |

The amino acid sequence identifiers of the VH and VL regions of exemplary rabbit anti-VISTA antibody clones are provided in Table 2 below.

TABLE 2
Additional Rabbit Anti-VISTA Antibodies

| | VH | | VL |
|---|---|---|---|
| Clone | SEQ ID NO: | Clone | SEQ ID NO: |
| 2H2 | 49 | 2L3 | 70 |
| 2H4 | 50 | | |
| 3H1 | 51 | 3L1 | 71 |
| 3H2 | 52 | | |
| 5H2 | 53 | 5L3 | 72 |
| 5H4 | 54 | | |
| 1H1 | 55 | 1L2 | 73 |
| 1H4 | 56 | | |
| 3A9-H1 | 58 | 3A9-L2 | 74 |
| 19B5-H2 | 59 | 19B5-L1 | 75 |
| 10D4H2 | 60 | 10D4-L1 | 76 |
| 32B7-H1 | 61 | 32B7-L1 | 77 |
| 9A3-H1 | 62 | 9A3-L1 | 78 |
| 34C3-H2 | 63 | 34C3-L1 | 79 |
| 5C12-H3 | 64 | 5C12-L1 | 80 |
| 18D3-H4 | 65 | 18D3-L2 | 81 |
| 15H1 | 66 | 15L1 | 82 |
| 4A2-H4 | 67 | 4A2-L3 | 83 |
| 5B6-H4 | 68 | 5B6-L1 | 84 |
| 6B5-H4 | 69 | 6B5-L1 | 85 |

The amino acid sequence identifiers of the VH regions, VL regions, CDRs, heavy chains, and light chains of exemplary humanized anti-VISTA antibodies 2D12-HZD3, 3A5-HZD, 14D8-HZD2, 14F1-HZD2, 29G7-HZD2, 29G7-HZD4, and 41A11-HZD2 are provided in Table 3 below.

TABLE 3
Humanized Anti-VISTA Antibodies

| | | | VHCD | VHCD | VHCD | | IgL | VLCD | VLCD | VLCD |
|---|---|---|---|---|---|---|---|---|---|---|
| | VH | IgH | R1 | R2 | R3 | VL | (kappa) | R1 | R2 | R3 |
| | SEQ | SEQ | SEQ | SEQ | SEQ | SEQ | SEQ | SEQ | SEQ | SEQ |
| | ID | ID | ID | ID | ID | ID | ID | ID | ID | ID |
| Clone | NO: | NO: | NO: | NO: | NO: | NO: | NO: | NO: | NO: | NO: |
| 2D12- | 86 | 100 | 3 | 4 | 5 | 87 | 101 | 6 | 7 | 8 |
| HZD3 | | | | | | | | | | |
| 3A5- | 88 | 102 | 11 | 12 | 13 | 89 | 103 | 14 | 15 | 16 |
| HZD | | | | | | | | | | |
| 14D8- | 90 | 104 | 19 | 20 | 21 | 91 | 105 | 22 | 23 | 24 |
| HZD2 | | | | | | | | | | |
| 14F1- | 92 | 106 | 27 | 28 | 29 | 93 | 107 | 30 | 31 | 32 |
| HZD2 | | | | | | | | | | |
| 29G7- | 94 | 108 | 35 | 36 | 37 | 95 | 109 | 38 | 39 | 40 |
| HZD2 | | | | | | | | | | |
| 29G7- | 96 | 110 | 35 | 36 | 37 | 97 | 111 | 38 | 39 | 40 |
| HZD4 | | | | | | | | | | |
| 41A11- | 98 | 112 | 43 | 44 | 45 | 99 | 113 | 46 | 47 | 48 |
| HZD2 | | | | | | | | | | |

| SEQ ID | |
|---|---|
| NO: | Amino Acid Sequences of anti-VISTA antibodies |
| 1 | LSLEESGGRLVTPGTPLTLTCTVSGIDLSSYAMGWVRQAPGKGLEYIGIISSSGSAFYA |
| | SWAKGRFTISKTSSTTVDLKMTSPTTEDTATYFCARHYAGYTTPYYFNFWGPGTLVTV |
| | SS |
| 2 | AYDMTQTPASVEVTVGGTVTIKCQASENIYSNLAWYQQKPGQPPKVLIY |
| | AASDLASGVPSRFKGSGSGTEYTLTISGVECADAATYYCQQGFSSSNVDNAFGGGTE |
| | VVVK |
| 3 | SYAMG |
| 4 | IISSSGSAFYASWAKG |
| 5 | HYAGYTTPYYFNF |
| 6 | QASENIYSNLA |
| 7 | AASDLAS |
| 8 | QQGFSSSNVDNA |
| 9 | QQLEQSGGGAEGGLVKPGGSLDLCCKASGFSLSSENWICWVRQAPGKGLEWIGCIC |
| | GANGGSAYHANWVNGRFTLSRDIDQSTDCLHLNSLTASDTAMYYCARAAGVSNAYYF |
| | DLWGPGTLVTVSS |
| 10 | DVVMTQTPSSVSAAVGGTVTIKCQASQYIYINLAWYQQKPGQRPKLLIYDASDLASGV |
| | PSRFKGSRSGTEFTLTISDLECADAATYYCQCTYGGSTYGNAFGGGTEVVVK |
| 11 | SENWIC |
| 12 | CICGANGGSAYHANWVNG |
| 13 | AAGVSNAYYFDL |
| 14 | QASQYIYINLA |
| 15 | DASDLAS |
| 16 | QCTYGGSTYGNA |
| 17 | QEQLEESGGGLVKPEGSLTLTCKASGFDLSSAYYMCWVRQAPGKGLEWIGCIATGSG |
| | STYYASWAKGRFTISKTSSTTVTLQATSLTAADTATLFCARAPYYSYDGYDYALNLWG |
| | PGTLVTVSS |
| 18 | IKMTQTPSSVSAVVGGTVTINCQASEDIVTYLAWYQQKPGHSPNLLIYAASTLASGVPS |
| | RFKGSGSGTQFTLTISDVQCDDAATYYCQQTYNYADTDNAFGGGTEVVVK |
| 19 | SAYYMC |
| 20 | CIATGSGSTYYASWAKG |
| 21 | APYYSYDGYDYALNL |
| 22 | QASEDIVTYLA |
| 23 | AASTLAS |
| 24 | QQTYNYADTDNA |
| 25 | QSLEESGGDLVKPGASLKLTCTASGFSFSSSYYMCWVRQAPGKGLEWVACFYAGFS |
| | GSTYYASWAKGRFTISKTSSTTVTLQMTSLTAADTATYFCAKGASTYGYTGYDYAGDY |
| | FILWGPGTLVTVSS |
| 26 | ALVMTQTPSSVSAAVGGTVTINCQASQNIYSNLAWYQQKPGQRPKLLIYAASNLASGV |
| | SSRFKGSGSGTEYTLTISDLECDDAATYYCQSAYYSSSADSYNAFGGGTEVVVK |
| 27 | SSYYMC |
| 28 | CFYAGFSGSTYYASWAKG |
| 29 | GASTYGYTGYDYAGDYFIL |
| 30 | QASQNIYSNLA |
| 31 | AASNLAS |
| 32 | QSAYYSSSADSYNA |
| 33 | QSLEESGGRLVTPGTPLTLTCTASGFSLSSYAMGWVRQAPGKGLEWIGIIIVSGNIYYA |
| | SWAKGQFTISKTSTTVDLKITSPTTEDTATYFCARGDGYTYGLWGQGTLVTVSS |
| 34 | DIVMTQTPASVEAAVGGTVTIKCQASQSVYSRYLAWFQQKPGQPPKLLIYSASTLASG |
| | VSSRFKGSGSGTEYTLTISGVQCDDAATYYCQGTFYIPDYIDGFGGGTEVVVK |
| 35 | SYAMG |
| 36 | IIIVSGNIYYASWAKG |
| 37 | GDGYTYGL |
| 38 | QASQSVYSRYLA |
| 39 | SASTLAS |
| 40 | QGTFYIPDYIDG |
| 41 | QSLEESGGDLVKPGASLKLTCTASGFSFSSSYYMCWVRQAPGKGLEWIGCFYAGFSG |
| | GTYYASWAKGRFTISKTSSTTVTLQMTGLTAADTATYFCAKGANTYGYTGYDYAGDYF |
| | TLWGPGTLVTVSS |
| 42 | ALVMTQTPSSVSAVVRGTVTINCQASQNIGSNLAWYQQKPGQRPKLLVYAASNLESG |
| | VPSRFKGSGSGTEFTLTISDLECDDAATYYCQSAYYSSVDSYNTFGGGTEVVVK |
| 43 | SSYYMC |
| 44 | CFYAGFSGGTYYASWAKG |
| 45 | GANTYGYTGYDYAGDYFTL |
| 46 | QASQNIGSNLA |
| 47 | AASNLES |
| 48 | QSAYYSSVDSYNT |
| 49 | QSVEESGGRLVTPGTPLTLTCTASGFSLSDYAMTWVRQAPGKGLEYIGIISAGGYTYY |
| | ASWATGRFTISKTSTTVDLKIPSPTAEDAATYFCARVGYADSSDIYKGFYLWGPGTLVT |
| | VSS |
| 50 | QSVEESGGRLVTPGTPLTLTCTASGFSLSDYAMTWVRQAPGKGLEYIGIISAGGYTYY |
| | ASWATGRFTISKTSTTVDLKIPSPTAEDAATYFCARVGYADSSDIYKGFYLWGPGTLAT |
| | VSS |
| 51 | QSLEESGGRLVTPGTPLTLTCTASGFFLNNYYMSWVRQAPGKGLEWIGLIYTSGSTLY |
| | ANWAKGRFTISRTSTTVDLKITSPTTEDTATYFCARYDNNVDSWLDLWGQGTLVTVSS |
| 52 | QSLEESGGHLVTPGTPPTLTCTASGFFLNNYYMSWVRQAPGKGLEWIGLIYTSGSTLY |
| | ANWAKGRFTISRTSTTVDLKITSPTTEDTATYFCARYDNNVDSWLDLWGQGTLVTVSS |
| 53 | QSLEESGGRLVTPGTPLTLTCTVSGIDLSSYAMAWVRQAPGEGLEWIGTISSSGHTYY |
| | PTWVKGRFTISKPSSTTVDLRMTSPTTEDTATYFCARGHYGYFGTVFALWGPGTLVTV |
| | SS |
| 54 | QSLEESGGRLVTPGTPLTLTCTVSGIDLSSYAMAWVRQAPGEGLEWIGTISSSGHTYY |
| | PTWVKGRFTISKPSSTTVDLRMTSPTTEDTATYFCARGHYGYFGTVFALWGPGTLVTV |
| | SS |
| 55 | QEQLVESGGGLVQPGGSLKLSCKASGFGFSSYGVSWVRQDPGKGLEWIGYIDPVLG |
| | SAAYATWVNGRFTISSHNAQNTLYLQLNSLTAADTATYFCARAGYPGYGYATNFNLW |
| | GPGTLVTVSS |
| 56 | QEQLVESGGGLVQPGGSLKLSCKAPGFDFSSYGVSWVRQDPGKGLEWIGYIDPVLGS |
| | AAYATWVNGRFTISSHNAQNTLYLQLNSLTAADTATYFCARAGYPGYGYATNFNLWG |
| | PGTLVTVSS |
| 58 | QSVEESGGRLVTPGTTLTLTCTVSGFSLSLYYMSWVRQAPGKGLEWIGMTYTGGSAY |
| | YASWAKGRFTISQTSTTVDLKMTTLTVADTATYFCVRGYLADYYGDRDNLWGPGTLVT |
| | VSS |
| 59 | QSVEESGGRLVTPGTPLTLTCTVSGFSLSSYWMSWVRQAPGEGLEWIGTIYTDGHTY |
| | YAKWAKGRFTISKTSTTVDLKMTSLTTADTATYFCARPWGVWGPGTLVTVSL |
| 60 | QSVEESGGRLVTPGTPLTLTCTVSGFSLISYWMIWVRQAPGEGLEWIGFINTGGSAYY |
| | ASWAKGRFTISRTSTTVDLKMTSLTTEDTATYFCARARYDSYITGDLWGPGTLVTVSS |
| 61 | QSVEESGGRLVTPGTPLTLTCTVSGFSLSSANMGWVRQAPGEGLEYIGLIDTDGTTYY |
| | ASWAKGRFTISRTSTTVDLKMTSLTTEDTATYFCARLRTGYGDLWGPGTLVAVSS |
| 62 | QSVEESGGRLVTPGTPLTLTCTASGFSLSDYAMTWVRQAPGKGLEYIGIISAGGYTYY |
| | ASWATGRFTISKTSTTVDLKIPSPTAEDAATYFCARVGYADSSDIYKGFYLWGPGTLVT |
| | VSS |
| 63 | QSVEESGGRLVTPGTPLTLTCTVSGFSLSSHAMSWVRQAPGKGLEWIGIIWTGGNTY |
| | YANWAKGRFTISKTSTTVDLKITSPIMEDTATYFCARYLNVEIGDVWGPGTLVTVSS |
| 64 | QSVEESGGRLVAPGTPLTLTCAVSGFSLSSYDMSWVRQAPGKGLEYIGVIGTGSTGP |
| | YYTSWAKGRFTISKTSTTVDLKITSPTTEDTATYFCARGWVKLDLWGPGTPVTVSS |
| 65 | QSVEESGGRLVTPGTPLTLACTVSGIDLSTNVMSWVRQAPGKGLEWIGIIFDSDITYYA |
| | NWAKGRFTISKTSTTVDLKFTSPTTEDTATYFCARYDTYGYSYSIDLWGPGTLVTVSS |
| 66 | QSLEESGGRLVTPGTPLTLTCTVSGIDLSDYAMGWVRQAPGKGLEYIAVISPIAYTYYA |
| | PWARGRFTISRASTTVDLKVTSPTTEDTATYFCARWAGTGWGYFNLWGPGTLVTVSS |
| 67 | QELVESGGGLVQPGESLKLSCKASGFDFSSYGVSWVRQAPGKGLEWVGYIDPVLGS |
| | AAHASWVNGRFTISSHNAQNTLYLQLNSLTAADTATYFCARAGYAGYGYGTGFNLWG |
| | PGTLVTVSS |
| 68 | QELVESGGGLVQAGESLKLSCKASGIDFSSYGISWVRQAPGKGLEWIAYIYPGNDITDY |
| | AHSVKGRFTISSDNAQNTVFLQMTSLTASDTATYFCARDAGYAGYGYPTGTYYFTLW |
| | GPGTLVTVSS |
| 69 | QELVESGGGLVQAGESLKLSCKASGIDFSSYGISWVRQAPGKGLGWIAYIYPGNDITD |
| | YANSVKGRFTISSDNAQNTVFLQMTSLTASDTATYFCARDAGYAGDGYPTGTYYFTL |
| | WGPGTLVTVSS |
| 70 | DVVMTQTPASVSEPVGGTVTIKCQASESISNYLAWYQQKPGQPPKLLIYLASTLASGVP |
| | SRFEGSRSGTEFTLTISDLECADAATYYCHCTDSIINYNFGGGTEVVVK |
| 71 | AYDMTQTPASVEVAVGGTVTIKCQASQSISSSYLAWYQQKPGQPPKLLIYVTSTLASG |
| | VSSRFKGSGSGTQFTLTISGVECADAATYYCQQGYSVSNIDNVFGGGTEVVVK |
| 72 | ADIVMTQTPSSVSAAVGGTVTINCQASESIQTYLAWYQQKPGQPPKLLIYDASDLASGV |
| | PSRFSGSGSGKQFTLTISGVQCDDAATYYCQNYYFSTNSNYGDVFGGGTEMVVK |
| 73 | ALVMTQTPSSVSAAVGGTVTINCQASQNIYSNLAWYQQKPGQRPKWYGASNLESGV |
| | PSRFKGSGSGIEYTLTISDLECDDAATYYCQTNYYSHSAPFGGGTEVVVK |
| 74 | IVMTQTPSSKSVPVGDTVTINCQASESVFSDDRLAWYQQKPGQPPKWYRASNLASG |
| | VPSRFSGSGSGTQFTLTINDVVCDDAATYYCAGYKGDSTDGVAFGGGTEVVVK |
| 75 | QVLTQTPSPVSAAVGGTVTISCQSSESVYSNNRLSWFQQKPGQPPKLLIYQASKLASG |
| | VPSRFSGSGSGTQFTLTISGVQCDDAATYYCLGGYDCSSADCDTFGGGTEVVVR |
| 76 | AVLTQTPSPVSAAVGGTVSISCQSSKSVSDNNWLSWYQQKPGQRPKLLIFYASTLASG |
| | VPSRFKGSGSGTQFTLTISDVQCDDAATYYCAGAYTNYNDNAFGGGTEVVVK |
| 77 | QAVVTQTPSSVSAAVGGTVTISCQSSQSVYNNNFLSWYQQKPGQPPKLLIYRASNLET |
| | GVSSRFSGSGSGTQFTLTISGVQCDDAATYYCAGYYSSGWYYTFGGGTEVVVK |
| 78 | DVVMTQTPASVSEPVGGTVTIKCQASESISNYLAWYQQKPGQPPKLLIYLASTLASGVP |
| | SRFEGSRSGTEFTLTISDLECADAATYYCHCTDSIINYNFGGGTEVVVK |
| 79 | QVLTQTPSSVSAAVGGTVTISCQSSQSVYKNNYLAWFQQKPGQPPKGLIYGASSLAS |
| | GVSSRFKGSGSGTQFTLTISDVQCVDAATYYCQGCYYTAGSGDNYAFGGGTEVVVR |
| 80 | QVLTQTPSPVSAAVGGTVTINCHSSQSVYNDNYLSWFQQKPGQPPKLLIYQASNLASG |
| | VPSRFKGSGSGTPFTLTIYDLECGDAATYYCAGGYNIGDSYSAFGGGTEVVVQ |
| 81 | AYDMTQTPASAEVAVGGTVTINCQASEDIYNLLAWYQQKPGQRPKWYRASTLASGV |
| | SSRFKGSGSGTDYTLTISGVQCDDAATYYCQQGHSSRNVDNAFGGGTEVVVK |
| 82 | AFELTQTPSSVSEPVGGTVTIKCQASESVSNYLAWYQQKPGQPPKWYGASTLESGV |
| | PSRFSGSGSGTEFTLTISDLECADAATYYCQEYDPSIDSGSVFGGGTELVVK |
| 83 | QVLTQTASPVSAAVGGTVTINCQASQN1YSNLAWYQQKPGQRPKWYGASNLESGVP |
| | SRFRGSGSGTDYTLTISDLECDDAATYYCQSAYYSTSADNPFGGGTEVVVK |
| 84 | ALVMTQTPSSVSAAVGGTVTINCQASQNIYNNLAWYQQKPGQRPKLLTYGASNLESG |
| | VPSRFKGSGSGTEYTLTISDLECDDAATYYCQSAYYSSSPDGAAFGGGTEVVVK |
| 85 | ALVMTQTPSSVSAPVGGTVTINCQASQSIYNNLAWYQQKPGQRPKWYGASNLESGV |
| | PSRFEGSGSGTEYTLTISDLECDDAATYYCQSAYYSSSPDGAAFGGGTEVVVK |
| 86 | EVQLVESGGGLVQPGGSLRLSCAASGIDLSSYAMGWVRQAPGKGLEWVGIISSSGSA |
| | FYASWAKGRFTISRDNSKNTLYLQMNSLRAEDTAVYYCARHYAGYTTPYYFNFWGQG |
| | TLVTVSS |
| 87 | AIQMTQSPSSLSASVGDRVTITCQASENIYSNLAWYQQKPGKAPKVLIYAASDLASGVP |
| | SRFSGSGSGTDFTLTISSLQPEDFATYYCQQGFSSSNVDNAFGGGTKVEIK |
| 88 | EVQLVESGGGLVQPGGSLRLSCAASGFSLSSENWICWVRQAPGKGLEWVGCICGAN |
| | GGSAYHANWVNGRFTISRDNSKNTLYLQMNSLRAEDTAVYYCARAAGVSNAYYFDL |
| | WGQGTLVTVSS |
| 89 | DIQMTQSPSSVSASVGDRVTITCQASQYIYINLAWYQQKPGKAPKWYDASDLASGVP |
| | SRFSGSGSGTDFTLTISSLQPEDFATYYCQCTYGGSTYGNAFGGGTKVEIK |
| 90 | EVQLVESGGGLVQPGGSLRLSCAASGFDLSSAYYMCWVRQAPGKGLEWVGCIATGS |
| | GSTYYASWAKGRFTISRDNSKNTLYLQMNSLRAEDTAVYYCARAPYYSYDGYDYALN |
| | LWGQGTLVTVSS |
| 91 | DIQMTQSPSSVSASVGDRVTITCQASEDIVTYLAWYQQKPGKAPKLLIYAASTLASGVP |
| | SRFSGSGSGTDFTLTISSLQPEDFATYYCQQTYNYADTDNAFGGGTKVEIK |
| 92 | EVQLLESGGGLVQPGGSLRLSCAASGFSFSSSYYMCWVRQAPGKGLEWVACFYAGF |
| | SGSTYYASWAKGRFTISRDNSKNTLYLQMNSLRAEDTAVYYCAKGASTYGYTGYDYA |
| | GDYFILWQPGTLVTVSS |
| 93 | DIQMTQSPSSVSASVGDRVTITCQASQNIYSNLAWYQQKPGKAPKLLIYAASNLASGV |
| | PSRFSGSGSGTDFTLTISSLQPEDFATYYCQSAYYSSSADSYNAFGGGTKVEIK |
| 94 | EVQLVESGGGLVQPGGSLRLSCAASGFSLSSYAMGWVRQAPGKGLEWVGIIIVSGNIY |
| | YASWAKGRFTISRDNSKNTLYLQMNSLRAEDTAVYYCARGDGYTYGLWGQGTLVTVS |
| | S |
| 95 | DIQMTQSPSTLSASVGDRVTITCQASQSVYSRYLAWYQQKPGKAPKWYSASTLASG |
| | VPSRFSGSGSGTEFTLTISSLQPDDFATYYCQGTFYIPDYIDGFGGGTKVEIK |
| 96 | EVQLVESGGGLVQPGGSLRLSCAASGFSLSSYAMGWVRQAPGKGLEWVGIIIVSGNIY |
| | YASWAKGRFTISRDNSKNTLYLQMNSLRAEDTAVYYCARGDGYTYGLWGQGTLVTVS |
| | S |
| 97 | EIVMTQSPATLSLSPGERATLSCQASQSVYSRYLAWYQQKPGQAPRLLIYSASTLASGI |
| | PARFSGSGSGTDFTLTISSLQPEDFAVYYCQGTFYIPDYIDGFGGGTKVEIK |
| 98 | EVQLLESGGGLVQPGGSLRLSCAASGFSFSSSYYMCWVRQAPGKGLEWVGCFYAGF |
| | SGGTYYASWAKGRFTISRDNSKNTLYLQMNSLRAEDTAVYYCAKGANTYGYTGYDYA |
| | GDYFTLWGQGTLVTVSS |
| 99 | DIQMTQSPSTLSASVGDRVTITCQASQNIGSNLAWYQQKPGKAPKLLIYAASNLESGVP |
| | SRFSGSGSGTEFTLTISSLQPDDFATYYCQSAYYSSVDSYNTFGGGTKVEIK |
| 100 | EVQLVESGGGLVQPGGSLRLSCAASGIDLSSYAMGWVRQAPGKGLEWVGIISSSGSA |
| | FYASWAKGRFTISRDNSKNTLYLQMNSLRAEDTAVYYCARHYAGYTTPYYFNFWGQG |
| | TLVTVSSASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPKSCDKTHTC |
| | PPCPAPELLGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQ |
| | PREPQVYTLPPSRDELTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 101 | AIQMTQSPSSLSASVGDRVTITCQASENIYSNLAWYQQKPGKAPKVLIYAASDLASGVP |
| | SRFSGSGSGTDFTLTISSLQPEDFATYYCQQGFSSSNVDNAFGGGTKVEIKRTVAAPS |
| | VFIFPPSDEQLKSGTASVVCLLNNFYPREAKVQWKVDNALQSGNSQESVTEQDSKDS |
| | TYSLSSTLTLSKADYEKHKVYACEVTHQGLSSPVTKSFNRGEC |
| 102 | EVQLVESGGGLVQPGGSLRLSCAASGFSLSSENWICWVRQAPGKGLEWVGCICGAN |
| | GGSAYHANWVNGRFTISRDNSKNTLYLQMNSLRAEDTAVYYCARAAGVSNAYYFDL |
| | WGQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTIS |
| | KAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGK |
| 103 | DIQMTQSPSSVSASVGDRVTITCQASQYIYINLAWYQQKPGKAPKWYDASDLASGVP |
| | SRFSGSGSGTDFTLTISSLQPEDFATYYCQCTYGGSTYGNAFGGGTKVEIKRTVAAPS |
| | VFIFPPSDEQLKSGTASVVCLLNNFYPREAKVQWKVDNALQSGNSQESVTEQDSKDS |
| | TYSLSSTLTLSKADYEKHKVYACEVTHQGLSSPVTKSFNRGEC |
| 104 | EVQLVESGGGLVQPGGSLRLSCAASGFDLSSAYYMCWVRQAPGKGLEWVGCIATGS |
| | GSTYYASWAKGRFTISRDNSKNTLYLQMNSLRAEDTAVYYCARAPYYSYDGYDYALN |
| | LWGQGTLVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKVEPKSC |
| | DKTHTCPPCPAPELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALPAPIEKTI |
| | SKAKGQPREPQVYTLPPSRDELTKNQVSLTCLVKGFYPSDIAVEWESNGQPENNYKT |
| | TPPVLDSDGSFFLYSKLTVDKSRWQQGNVFSCSVMHEALHNHYTQKSLSLSPGK |
| 105 | DIQMTQSPSSVSASVGDRVTITCQASEDIVTYLAWYQQKPGKAPKLLIYAASTLASGVP |
| | SRFSGSGSGTDFTLTISSLQPEDFATYYCQQTYNYADTDNAFGGGTKVEIKRTVAAPS |
| | VFIFPPSDEQLKSGTASVVCLLNNFYPREAKVQWKVDNALQSGNSQESVTEQDSKDS |
| | TYSLSSTLTLSKADYEKHKVYACEVTHQGLSSPVTKSFNRGEC |
| 106 | EVQLLESGGGLVQPGGSLRLSCAASGFSFSSSYYMCWVRQAPGKGLEWVACFYAGF |
| | SGSTYYASWAKGRFTISRDNSKNTLYLQMNSLRAEDTAVYYCAKGASTYGYTGYDYA |
| | GDYFILWQPGTLVTVSSASTKGPSVFPLAPSSKSTSGGTAALGCLVKDYFPEPVTVSW |
| | NSGALTSGVHTFPAVLQSSGLYSLSSVVTVPSSSLGTQTYICNVNHKPSNTKVDKKVE |
| | PKSCDKTHTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQKSLSLSPG |
| | K |
| 107 | DIQMTQSPSSVSASVGDRVTITCQASQNIYSNLAWYQQKPGKAPKLLIYAASNLASGV |
| | PSRFSGSGSGTDFTLTISSLQPEDFATYYCQSAYYSSSADSYNAFGGGTKVEIKRTVA |
| | APSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |
| 108 | EVQLVESGGGLVQPGGSLRLSCAASGFSLSSYAMGWVRQAPGKGLEWVGIIIVSGNIY |
| | YASWAKGRFTISRDNSKNTLYLQMNSLRAEDTAVYYCARGDGYTYGLWGQGTLVTVS |
| | SASTKGPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKKVEPKSCDKTHTCPPCPAP |
| | ELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| 109 | DIQMTQSPSTLSASVGDRVTITCQASQSVYSRYLAWYQQKPGKAPKWYSASTLASG |
| | VPSRFSGSGSGTEFTLTISSLQPDDFATYYCQGTFYIPDYIDGFGGGTKVEIKRTVAAP |
| | SVFIFPPSDEQLKSGTASVVCLLNNFYPREAKVQWKVDNALQSGNSQESVTEQDSKD |
| | STYSLSSTLTLSKADYEKHKVYACEVTHQGLSSPVTKSFNRGEC |
| 110 | EVQLVESGGGLVQPGGSLRLSCAASGFSLSSYAMGWVRQAPGKGLEWVGIIIVSGNIY |
| | YASWAKGRFTISRDNSKNTLYLQMNSLRAEDTAVYYCARGDGYTYGLWGQGTLVTVS |
| | SASTKGPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKKVEPKSCDKTHTCPPCPAP |
| | ELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| 111 | EIVMTQSPATLSLSPGERATLSCQASQSVYSRYLAWYQQKPGQAPRLLIYSASTLASGI |
| | PARFSGSGSGTDFTLTISSLQPEDFAVYYCQGTFYIPDYIDGFGGGTKVEIKRTVAAPS |
| | VFIFPPSDEQLKSGTASVVCLLNNFYPREAKVQWKVDNALQSGNSQESVTEQDSKDS |
| | TYSLSSTLTLSKADYEKHKVYACEVTHQGLSSPVTKSFNRGEC |
| 112 | EVQLLESGGGLVQPGGSLRLSCAASGFSFSSSYYMCWVRQAPGKGLEWVGCFYAGF |
| | SGGTYYASWAKGRFTISRDNSKNTLYLQMNSLRAEDTAVYYCAKGANTYGYTGYDYA |
| | GDYFTLWGQGTLVTVSSASTKGPSVFPLAPSSKSTSGGTAALGCLVKDYFPEPVTVS |
| | WNSGALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSLSP |
| | GK |
| 113 | DIQMTQSPSTLSASVGDRVTITCQASQNIGSNLAWYQQKPGKAPKLLIYAASNLESGVP |
| | SRFSGSGSGTEFTLTISSLQPDDFATYYCQSAYYSSVDSYNTFGGGTKVEIKRTVAAP |
| | SVFIFPPSDEQLKSGTASVVCLLNNFYPREAKVQWKVDNALQSGNSQESVTEQDSKD |
| | STYSLSSTLTLSKADYEKHKVYACEVTHQGLSSPVTKSFNRGEC |

### DETAILED DESCRIPTION

The present disclosure relates to antibodies and antigen-binding fragments thereof the specifically bind to V-domain Ig suppressor of T cell activation (VISTA), in particular antibodies having specific epitopic specificity and functional properties. One embodiment of the disclosure encompasses specific humanized antibodies and fragments thereof capable of binding to VISTA, blocking VISTA binding with a ligand and/or counter receptor and inhibiting induced downstream cell signaling and biological effects. In some embodiments, an anti-VISTA antibody, or antigen-binding fragment thereof, is a VISTA antagonist or inhibitor. An antagonist of VISTA enhances immune responses by blocking or otherwise reducing the immunosuppressive actions of VISTA. VISTA antagonist antibodies of the disclosure are useful in the treatment and prevention of, e.g., cancers, especially VISTA-expressing cancers, and infectious diseases. In some embodiments, an anti-VISTA antibody, or antigen-binding fragment thereof, is a VISTA agonist or activator. An agonist of VISTA suppresses immune responses by increasing the immunosuppressive actions of VISTA. VISTA agonist antibodies of the disclosure are useful in the treatment and prevention of, e.g., autoimmune diseases and disorders, transplants (e.g., graft-versus-host disease and transplant rejection), and inflammatory diseases and disorders.

Embodiments of the disclosure pertain to the use of anti-VISTA antibodies or antigen-binding fragments thereof for the diagnosis, assessment and treatment of diseases and disorders associated with VISTA or aberrant expression thereof. The subject antibodies are used in the treatment or prevention of cancer among other diseases. Other embodiments of the disclosure pertain to the use of anti-VISTA antibodies or antigen-binding fragments thereof for the diagnosis, assessment and treatment of diseases and disorders associated with aberrant or unwanted inflammatory T cell responses.

The practice of the present disclosure will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Current Protocols in Molecular Biology or Current Protocols in Immunology, John Wiley & Sons, New York, N.Y. (2009); Ausubel et al., Short Protocols in Molecular Biology, 3^{rd} ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

As used in this specification and the appended claims, the singular forms “a,” “an” and “the” include plural references unless the content clearly dictates otherwise.

An “antagonist” refers to an agent (e.g., antibody) that interferes with or otherwise reduces the physiological action of another agent or molecule. In some instances, the antagonist specifically binds to the other agent or molecule. Included are full and partial antagonists.

An “agonist” refers to an agent (e.g., antibody) that increases or enhances the physiological action of another agent or molecule. In some instances, the agonist specifically binds to the other agent or molecule. Included are full and partial agonists.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 1.5, 2, 3, 4, 5, 6, 7, 8, 9, 10, 15, 20, 30, 40, 50, 60, 70, 80, 90, 100 or more times (e.g., 500, 1000 times) (including all integers and ranges in between e.g., 1.5, 1.6, 1.7. 1.8, etc.) the amount produced by no composition (e.g., the absence of agent) or a control composition. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease (including all integers and ranges in between) in the amount produced by no composition (e.g., the absence of an agent) or a control composition. Examples of comparisons and “statistically significant” amounts are described herein.

“Substantially” or “essentially” means nearly totally or completely, for instance, 95%, 96%, 97%, 98%, 99% or greater of some given quantity.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Each embodiment in this specification is to be applied mutatis mutandis to every other embodiment unless expressly stated otherwise.

Standard techniques may be used for recombinant DNA, oligonucleotide synthesis, and tissue culture and transformation (e.g., electroporation, lipofection).

Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

Embodiments of the present disclosure relate to antibodies that bind to VISTA. In particular, the antibodies described herein specifically bind to VISTA with unexpectedly high affinity, block binding of VISTA to antigen presenting cells (APCs) and/or T cells, block VISTA activity and have therapeutic utility for the treatment of diseases associated with aberrant expression of VISTA. The antibodies described herein also have advantageous properties such as the ability to inhibit a variety of VISTA-mediated biological effects (e.g., inhibition of T cell proliferation, inhibition of T cell activation, and other VISTA-mediated effects known to the skilled person). The ligand and/or counter-receptor for VISTA is still unknown. However, the anti-VISTA antibodies described herein may block binding of VISTA to its ligand and/or counter-receptor. In one embodiment, use of an anti-VISTA antibody results in T cell proliferation. In one embodiment, use of an anti-VISTA antibody results in T cell activation. In one embodiment, use of an anti-VISTA antibody results in increased secretion of IFN-γ. In one embodiment, use of an anti-VISTA antibody results in increased secretion of IL-2. In one embodiment, use of an anti-VISTA antibody results in increased expression of MHC II. In some embodiments, use of an anti-VISTA antibody results in increased activation of natural killer (NK) cells. In some embodiments, use of an anti-VISTA antibody results in increased activation of monocytes/macrophages. In some embodiments, use of an anti-VISTA antibody results in increased cytokine production, for example, increased production of a cytokine is selected from one or more of IFN-gamma, IL-6, IL-1ra, IL-1a, IL-8, MIP-1a, MIP-1b IP-10, TNF-alpha and MCP-1.

Sequences of illustrative antibodies, or antigen-binding fragments, or complementarity determining regions (CDRs) thereof, are set forth in SEQ ID NOs:1-113.

As is well known in the art, an antibody is an immunoglobulin molecule capable of specific binding to a target, such as a carbohydrate, polynucleotide, lipid, polypeptide, etc., through at least one epitope recognition site, located in the variable region of the immunoglobulin molecule. As used herein, the term encompasses not only intact polyclonal or monoclonal antibodies, but also fragments thereof (such as dAb, Fab, Fab′, F(ab′)₂, Fv), single chain (scFv), synthetic variants thereof, naturally occurring variants, fusion proteins comprising an antibody portion with an antigen-binding fragment of the required specificity, humanized antibodies, chimeric antibodies, and any other modified configuration of the immunoglobulin molecule that comprises an antigen-binding site or fragment (epitope recognition site) of the required specificity. “Diabodies”, multivalent or multispecific fragments constructed by gene fusion (WO94/13804; P. Holliger et al., Proc. Natl. Acad. Sci. USA 90 6444-6448, 1993) are also a particular form of antibody contemplated herein. Minibodies comprising a scFv joined to a CH3 domain are also included herein (S. Hu et al., Cancer Res., 56, 3055-3061, 1996). See e.g., Ward, E. S. et al., Nature 341, 544-546 (1989); Bird et al., Science, 242, 423-426, 1988; Huston et al., PNAS USA, 85, 5879-5883, 1988); PCT/US92/09965; WO94/13804; P. Holliger et al., Proc. Natl. Acad. Sci. USA 90 6444-6448, 1993; Y. Reiter et al., Nature Biotech, 14, 1239-1245, 1996; S. Hu et al., Cancer Res., 56, 3055-3061, 1996.

The term “antigen-binding fragment” as used herein refers to a polypeptide fragment that contains at least one CDR of an immunoglobulin heavy and/or light chains that binds to the antigen of interest, in particular to VISTA. In this regard, an antigen-binding fragment of the herein described antibodies may comprise 1, 2, 3, 4, 5, or all 6 CDRs of a VH and VL sequence set forth herein from antibodies that bind VISTA. An antigen-binding fragment of the VISTA-specific antibodies described herein is capable of binding to VISTA. In certain embodiments, an antigen-binding fragment or an antibody comprising an antigen-binding fragment, prevents or inhibits VISTA binding to T cells and/or APCs and subsequent signaling events. In certain embodiments, the antigen-binding fragment binds specifically to and/or inhibits or modulates the biological activity of human VISTA. In certain embodiments, the antigen-binding fragment binds specifically to and/or enhances or upregulates the biological activity of human VISTA.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes.

The term “epitope” includes any determinant, preferably a polypeptide determinant, capable of specific binding to an immunoglobulin or T-cell receptor. An epitope is a region of an antigen that is bound by an antibody. In certain embodiments, epitope determinants include chemically active surface groupings of molecules such as amino acids, sugar side chains, phosphoryl or sulfonyl, and may in certain embodiments have specific three-dimensional structural characteristics, and/or specific charge characteristics. In certain embodiments, an antibody is said to specifically bind an antigen when it preferentially recognizes its target antigen in a complex mixture of proteins and/or macromolecules. An antibody is said to specifically bind an antigen when the equilibrium dissociation constant is ≤10⁻⁷ or 10⁻⁸ M. In some embodiments, the equilibrium dissociation constant may be ≤10⁻⁹ M or ≤10⁻¹° M.

In certain embodiments, antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

The structures and locations of immunoglobulin variable domains may be determined by reference to Kabat, E. A. et al., Sequences of Proteins of Immunological Interest. 4th Edition. US Department of Health and Human Services. 1987, and updates thereof, now available on the Internet (immuno.bme.nwu.edu).

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)₂, Fv), single chain (scFv), variants thereof, fusion proteins comprising an antigen-binding portion, humanized monoclonal antibodies, chimeric monoclonal antibodies, and any other modified configuration of the immunoglobulin molecule that comprises an antigen-binding fragment (epitope recognition site) of the required specificity and the ability to bind to an epitope. It is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody”.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment which comprises both antigen-binding sites. An Fv fragment for use according to certain embodiments of the present disclosure can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent V_{H}::V_{L} heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

In certain embodiments, single chain Fv or scFv antibodies are contemplated. For example, Kappa bodies (III et al., Prot. Eng. 10: 949-57 (1997); minibodies (Martin et al., EMBO J 13: 5305-9 (1994); diabodies (Holliger et al., PNAS 90: 6444-8 (1993); or Janusins (Traunecker et al., EMBO J 10: 3655-59 (1991) and Traunecker et al., Int. J. Cancer Suppl. 7: 51-52 (1992), may be prepared using standard molecular biology techniques following the teachings of the present application with regard to selecting antibodies having the desired specificity. In still other embodiments, bispecific or chimeric antibodies may be made that encompass the ligands of the present disclosure. For example, a chimeric antibody may comprise CDRs and framework regions from different antibodies, while bispecific antibodies may be generated that bind specifically to VISTA through one binding domain and to a second molecule through a second binding domain. These antibodies may be produced through recombinant molecular biological techniques or may be physically conjugated together.

A single chain Fv (scFv) polypeptide is a covalently linked V_{H}::V_{L} heterodimer which is expressed from a gene fusion including V_{H}- and V_{L}-encoding genes linked by a peptide-encoding linker. Huston et al. (1988) Proc. Nat. Acad. Sci. USA 85(16):5879-5883. A number of methods have been described to discern chemical structures for converting the naturally aggregated—but chemically separated—light and heavy polypeptide chains from an antibody V region into an scFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

In certain embodiments, a VISTA binding antibody as described herein is in the form of a diabody. Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO94/13804).

A dAb fragment of an antibody consists of a VH domain (Ward, E. S. et al., Nature 341, 544-546 (1989)).

Where bispecific antibodies are to be used, these may be conventional bispecific antibodies, which can be manufactured in a variety of ways (Holliger, P. and Winter G. Current Opinion Biotechnol. 4, 446-449 (1993)), e.g. prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. Bispecific whole antibodies may be made by knobs-into-holes engineering (J. B. B. Ridgeway et al., Protein Eng., 9, 616-621, 1996).

In certain embodiments, the antibodies described herein may be provided in the form of a UniBody®. A UniBody® is an IgG4 antibody with the hinge region removed (see GenMab Utrecht, The Netherlands; see also, e.g., US20090226421). This proprietary antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells. For certain cancer cell surface antigens, this univalent binding may not stimulate the cancer cells to grow as may be seen using bivalent antibodies having the same antigen specificity, and hence UniBody® technology may afford treatment options for some types of cancer that may be refractory to treatment with conventional antibodies. The small size of the UniBody® can be a great benefit when treating some forms of cancer, allowing for better distribution of the molecule over larger solid tumors and potentially increasing efficacy.

In certain embodiments, the antibodies of the present disclosure may take the form of a Nanobody®. Nanobodies® are encoded by single genes and are efficiently produced in almost all prokaryotic and eukaryotic hosts e.g. E. coli (see e.g. U.S. Pat. No. 6,765,087), molds (for example Aspergillus or Trichoderma) and yeast (for example Saccharomyces, Kluyvermyces, Hansenula or Pichia (see e.g. U.S. Pat. No. 6,838,254). The production process is scalable and multi-kilogram quantities of Nanobodies® have been produced. Nanobodies may be formulated as a ready-to-use solution having a long shelf life. The Nanoclone® method (see, e.g., WO 06/079372) is a proprietary method for generating Nanobodies against a desired target, based on automated high-throughput selection of B-cells.

In certain embodiments, the anti-VISTA antibodies or antigen-binding fragments thereof as disclosed herein are humanized. This refers to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild type or modified by one or more amino acid substitutions. This eliminates the constant region as an immunogen in human individuals, but the possibility of an immune response to the foreign variable region remains (LoBuglio, A. F. et al., (1989) Proc Natl Acad Sci USA 86:4220-4224; Queen et al., PNAS (1988) 86:10029-10033; Riechmann et al., Nature (1988) 332:323-327). Illustrative methods for humanization of the anti-VISTA antibodies disclosed herein include the methods described in U.S. Pat. No. 7,462,697. Illustrative humanized antibodies according to certain embodiments comprise the humanized sequences provided in SEQ ID NOs:86-113.

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato, K., et al., (1993) Cancer Res 53:851-856. Riechmann, L., et al., (1988) Nature 332:323-327; Verhoeyen, M., et al., (1988) Science 239:1534-1536; Kettleborough, C. A., et al., (1991) Protein Engineering 4:773-3783; Maeda, H., et al., (1991) Human Antibodies Hybridoma 2:124-134; Gorman, S. D., et al., (1991) Proc Natl Acad Sci USA 88:4181-4185; Tempest, P. R., et al., (1991) Bio/Technology 9:266-271; Co, M. S., et al., (1991) Proc Natl Acad Sci USA 88:2869-2873; Carter, P., et al., (1992) Proc Natl Acad Sci USA 89:4285-4289; and Co, M. S. et al., (1992) J Immunol 148:1149-1154. In some embodiments, humanized antibodies preserve all CDR sequences (for example, a humanized rabbit antibody which contains all six CDRs from the rabbit antibody). In other embodiments, humanized antibodies have one or more CDRs (one, two, three, four, five, six) which are altered with respect to the original antibody, which are also termed one or more CDRs “derived from” one or more CDRs from the original antibody.

In certain embodiments, the antibodies of the present disclosure may be chimeric antibodies. In this regard, a chimeric antibody is comprised of an antigen-binding fragment of an anti-VISTA antibody operably linked or otherwise fused to a heterologous Fc portion of a different antibody. In certain embodiments, the heterologous Fc domain is of human origin. In other embodiments, the heterologous Fc domain may be from a different Ig class from the parent antibody, including IgA (including subclasses IgA1 and IgA2), IgD, IgE, IgG (including subclasses IgG1, IgG2, IgG3, and IgG4), and IgM. In further embodiments, the heterologous Fc domain may be comprised of CH2 and CH3 domains from one or more of the different Ig classes. As noted above with regard to humanized antibodies, the anti-VISTA antigen-binding fragment of a humanized antibody may comprise only one or more of the CDRs of the antibodies described herein (e.g., 1, 2, 3, 4, 5, or 6 CDRs of the antibodies described herein), or may comprise an entire variable domain (VL, VH or both).

In certain embodiments, a VISTA-binding antibody comprises one or more of the CDRs of the antibodies described herein. In this regard, it has been shown in some cases that the transfer of only the VHCDR3 of an antibody can be performed while still retaining desired specific binding (Barbas et al., PNAS (1995) 92: 2529-2533). See also, McLane et al., PNAS (1995) 92:5214-5218, Barbas et al., J. Am. Chem. Soc. (1994) 116:2161-2162.

Marks et al (Bio/Technology, 1992, 10:779-783) describe methods of producing repertoires of antibody variable domains in which consensus primers directed at or adjacent to the 5′ end of the variable domain area are used in conjunction with consensus primers to the third framework region of human VH genes to provide a repertoire of VH variable domains lacking a CDR3. Marks et al further describe how this repertoire may be combined with a CDR3 of a particular antibody. Using analogous techniques, the CDR3-derived sequences of the presently described antibodies may be shuffled with repertoires of VH or VL domains lacking a CDR3, and the shuffled complete VH or VL domains combined with a cognate VL or VH domain to provide an antibody or antigen-binding fragment thereof that binds VISTA. The repertoire may then be displayed in a suitable host system such as the phage display system of WO 92/01047 so that suitable antibodies or antigen-binding fragments thereof may be selected. A repertoire may consist of at least from about 10⁴ individual members and upwards by several orders of magnitude, for example, to about from 10⁶ to 10⁸ or 10¹⁰ or more members. Analogous shuffling or combinatorial techniques are also disclosed by Stemmer (Nature, 1994, 370:389-391), who describes the technique in relation to a β-lactamase gene but observes that the approach may be used for the generation of antibodies.

A further alternative is to generate novel VH or VL regions carrying one or more CDR-derived sequences described herein using random mutagenesis of one or more selected VH and/or VL genes to generate mutations within the entire variable domain. Such a technique is described by Gram et al (1992, Proc. Natl. Acad. Sci., USA, 89:3576-3580), who used error-prone PCR. Another method which may be used is to direct mutagenesis to CDR regions of VH or VL genes. Such techniques are disclosed by Barbas et al., (1994, Proc. Natl. Acad. Sci., USA, 91:3809-3813) and Schier et al (1996, J. Mol. Biol. 263:551-567).

In certain embodiments, a specific VH and/or VL of the antibodies described herein may be used to screen a library of the complementary variable domain to identify antibodies with desirable properties, such as increased affinity for VISTA. Such methods are described, for example, in Portolano et al., J. Immunol. (1993) 150:880-887; Clarkson et al., Nature (1991) 352:624-628.

Other methods may also be used to mix and match CDRs to identify antibodies having desired binding activity, such as binding to VISTA. For example: Klimka et al., British Journal of Cancer (2000) 83: 252-260, describe a screening process using a mouse VL and a human VH library with CDR3 and FR4 retained from the mouse VH. After obtaining antibodies, the VH was screened against a human VL library to obtain antibodies that bound antigen. Beiboer et al., J. Mol. Biol. (2000) 296:833-849 describe a screening process using an entire mouse heavy chain and a human light chain library. After obtaining antibodies, one VL was combined with a human VH library with the CDR3 of the mouse retained. Antibodies capable of binding antigen were obtained. Rader et al., PNAS (1998) 95:8910-8915 describe a process similar to Beiboer et al above.

These just-described techniques are, in and of themselves, known as such in the art. The skilled person will, however, be able to use such techniques to obtain antibodies or antigen-binding fragments thereof according to several embodiments described herein, using routine methodology in the art.

Also disclosed herein is a method for obtaining an antibody antigen binding domain specific for VISTA antigen, the method comprising providing by way of addition, deletion, substitution or insertion of one or more amino acids in the amino acid sequence of a VH domain set out herein a VH domain which is an amino acid sequence variant of the VH domain, optionally combining the VH domain thus provided with one or more VL domains, and testing the VH domain or VH/VL combination or combinations to identify a specific binding member or an antibody antigen binding domain specific for VISTA and optionally with one or more desired properties. The VL domains may have an amino acid sequence which is substantially as set out herein. An analogous method may be employed in which one or more sequence variants of a VL domain disclosed herein are combined with one or more VH domains.

An epitope that “specifically binds” or “preferentially binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a VISTA epitope is an antibody that binds one VISTA epitope with greater affinity, avidity, more readily, and/or with greater duration than it binds to other VISTA epitopes or non-VISTA epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

Immunological binding generally refers to the non-covalent interactions of the type which occur between an immunoglobulin molecule and an antigen for which the immunoglobulin is specific, for example by way of illustration and not limitation, as a result of electrostatic, ionic, hydrophilic and/or hydrophobic attractions or repulsion, steric forces, hydrogen bonding, van der Waals forces, and other interactions. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant K_{d}. See, generally, Davies et al. (1990) Annual Rev. Biochem. 59:439-473.

In certain embodiments, the anti-VISTA antibodies described herein have an affinity of about 100, 150, 155, 160, 170, 175, 180, 185, 190, 191, 192, 193, 194, 195, 196, 197, 198 or 199 picomolar, and in some embodiments, the antibodies may have even higher affinity for VISTA.

The term “immunologically active”, with reference to an epitope being or “remaining immunologically active”, refers to the ability of an antibody (e.g., anti-VISTA antibody) to bind to the epitope under different conditions, for example, after the epitope has been subjected to reducing and denaturing conditions.

An antibody or antigen-binding fragment thereof according to certain preferred embodiments of the present application may be one that competes for binding to VISTA with any antibody described herein which both (i) specifically binds to the antigen and (ii) comprises a VH and/or VL domain disclosed herein, or comprises a VH CDR3 disclosed herein, or a variant of any of these. Competition between antibodies may be assayed easily in vitro, for example using ELISA and/or by tagging a specific reporter molecule to one antibody which can be detected in the presence of other untagged antibodies, to enable identification of specific antibodies which bind the same epitope or an overlapping epitope. Thus, there is provided herein a specific antibody or antigen-binding fragment thereof, comprising a human antibody antigen-binding site which competes with an antibody described herein that binds to VISTA.

In this regard, as used herein, the terms “competes with”, “inhibits binding” and “blocks binding” (e.g., referring to inhibition/blocking of binding of a ligand and/or counter-receptor to VISTA or referring to inhibition/blocking of binding of an anti-VISTA antibody to VISTA) are used interchangeably and encompass both partial and complete inhibition/blocking. The ligand and/or counter-receptor of VISTA is still undefined (Nowak et al. Immunological Reviews 2017 276:66-79). The inhibition/blocking of a ligand and/or counter-receptor to VISTA preferably reduces or alters the normal level or type of cell signaling that occurs when a ligand and/or counter-receptor binds to VISTA without inhibition or blocking. Inhibition and blocking are also intended to include any measurable decrease in the binding of a ligand and/or counter-receptor to VISTA when in contact with an anti-VISTA antibody as disclosed herein as compared to the ligand not in contact with an anti-VISTA antibody, e.g., the blocking of a ligand and/or counter-receptor to VISTA by at least about 10%, 20%, 30%, 40%, 50%, 60%, 65%, 70%, 75%, 80%, 85%, 90%, 91%, 92%, 93%, 94%, 95%, 96%, 97%, 98%, 99%, or 100%.

The constant regions of immunoglobulins show less sequence diversity than the variable regions, and are responsible for binding a number of natural proteins to elicit important biochemical events. In humans there are five different classes of antibodies including IgA (which includes subclasses IgA1 and IgA2), IgD, IgE, IgG (which includes subclasses IgG1, IgG2, IgG3, and IgG4), and IgM. The distinguishing features between these antibody classes are their constant regions, although subtler differences may exist in the V region.

The Fc region of an antibody interacts with a number of Fc receptors and ligands, imparting an array of important functional capabilities referred to as effector functions. In one embodiment, an anti-VISTA antibody comprises an Fc region. For IgG the Fc region comprises Ig domains CH2 and CH3 and the N-terminal hinge leading into CH2. An important family of Fc receptors for the IgG class are the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290). In humans this protein family includes FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65). These receptors typically have an extracellular domain that mediates binding to Fc, a membrane spanning region, and an intracellular domain that may mediate some signaling event within the cell. These receptors are expressed in a variety of immune cells including monocytes, macrophages, neutrophils, dendritic cells, eosinophils, mast cells, platelets, B cells, large granular lymphocytes, Langerhans' cells, natural killer (NK) cells, and T cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack.

The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290). The cell-mediated reaction wherein nonspecific cytotoxic cells that express FcγRs recognize bound antibody on a target cell and subsequently cause phagocytosis of the target cell is referred to as antibody dependent cell-mediated phagocytosis (ADCP). All FcγRs bind the same region on Fc, at the N-terminal end of the Cg2 (CH2) domain and the preceding hinge. This interaction is well characterized structurally (Sondermann et al., 2001, J Mol Biol 309:737-749), and several structures of the human Fc bound to the extracellular domain of human FcγRIIIb have been solved (pdb accession code 1E4K) (Sondermann et al., 2000, Nature 406:267-273.) (pdb accession codes 1IIS and 1IIX) (Radaev et al., 2001, J Biol Chem 276:16469-16477.)

The different IgG subclasses have different affinities for the FcγRs, with IgG1 and IgG3 typically binding substantially better to the receptors than IgG2 and IgG4 (Jefferis et al., 2002, Immunol Lett 82:57-65). All FcγRs bind the same region on IgG Fc, yet with different affinities: the high affinity binder FcγRI has a K_{d} for IgG1 of 10⁻⁸ M⁻¹, whereas the low affinity receptors FcγRII and FcγRIII generally bind at 10⁻⁶ and 10⁻⁵ respectively. The extracellular domains of FcγRIIIa and FcγRIIIb are 96% identical; however, FcγRIIIb does not have an intracellular signaling domain. Furthermore, whereas FcγRI, FcγRIIa/c, and FcγRIIIa are positive regulators of immune complex-triggered activation, characterized by having an intracellular domain that has an immunoreceptor tyrosine-based activation motif (ITAM), FcγRIIb has an immunoreceptor tyrosine-based inhibition motif (ITIM) and is therefore inhibitory. Thus the former are referred to as activation receptors, and FcγRIIb is referred to as an inhibitory receptor. The receptors also differ in expression pattern and levels on different immune cells. Yet another level of complexity is the existence of a number of FcγR polymorphisms in the human proteome. A particularly relevant polymorphism with clinical significance is V158/F158 FcγRIIIa. Human IgG1 binds with greater affinity to the V158 allotype than to the F158 allotype. This difference in affinity, and presumably its effect on ADCC and/or ADCP, has been shown to be a significant determinant of the efficacy of the anti-CD20 antibody rituximab (Rituxan®, a registered trademark of IDEC Pharmaceuticals Corporation). Patients with the V158 allotype respond favorably to rituximab treatment; however, patients with the lower affinity F158 allotype respond poorly (Cartron et al., 2002, Blood 99:754-758). Approximately 10-20% of humans are V158/V158 homozygous, 45% are V158/F158 heterozygous, and 35-45% of humans are F158/F158 homozygous (Lehrnbecher et al., 1999, Blood 94:4220-4232; Cartron et al., 2002, Blood 99:754-758). Thus 80-90% of humans are poor responders, that is, they have at least one allele of the F158 FcγRIIIa.

The Fc region is also involved in activation of the complement cascade. In the classical complement pathway, C1 binds with its C1q subunits to Fc fragments of IgG or IgM, which has formed a complex with antigen(s). In certain embodiments, modifications to the Fc region comprise modifications that alter (either enhance or decrease) the ability of a VISTA-specific antibody as described herein to activate the complement system (see e.g., U.S. Pat. No. 7,740,847). To assess complement activation, a complement-dependent cytotoxicity (CDC) assay may be performed (See, e.g., Gazzano-Santoro et al., J. Immunol. Methods, 202:163 (1996)).

Thus in certain embodiments, the present disclosure provides anti-VISTA antibodies having a modified Fc region with altered functional properties, such as reduced or enhanced CDC, ADCC, or ADCP activity, or enhanced binding affinity for a specific FcγR or increased serum half-life. Other modified Fc regions contemplated herein are described, for example, in issued U.S. Pat. Nos. 7,317,091; 7,657,380; 7,662,925; 6,538,124; 6,528,624; 7,297,775; 7,364,731; Published U.S. Applications US2009092599; US20080131435; US20080138344; and published International Applications WO2006/105338; WO2004/063351; WO2006/088494; WO2007/024249.

Thus, in certain embodiments, antibody variable domains with the desired binding specificities are fused to immunoglobulin constant domain sequences. In certain embodiments, the fusion is with an Ig heavy chain constant domain, comprising at least part of the hinge, C_{H}2, and C_{H}3 regions. It is preferred to have the first heavy-chain constant region (C_{H}1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

Antibodies of the present disclosure (and antigen-binding fragments and variants thereof) may also be modified to include an epitope tag or label, e.g., for use in purification or diagnostic applications. There are many linking groups known in the art for making antibody conjugates, including, for example, those disclosed in U.S. Pat. No. 5,208,020 or EP Patent 0 425 235 B1, and Chari et al., Cancer Research 52: 127-131 (1992). The linking groups include disulfide groups, thioether groups, acid labile groups, photolabile groups, peptidase labile groups, or esterase labile groups, as disclosed in the above-identified patents, disulfide and thioether groups being preferred.

In another contemplated embodiment, a VISTA-specific antibody as described herein may be conjugated or operably linked to another therapeutic compound, referred to herein as a conjugate. The conjugate may be a cytotoxic agent, a chemotherapeutic agent, a cytokine, an anti-angiogenic agent, a tyrosine kinase inhibitor, a toxin, a radioisotope, or other therapeutically active agent. Chemotherapeutic agents, cytokines, anti-angiogenic agents, tyrosine kinase inhibitors, and other therapeutic agents have been described above, and all of these aforementioned therapeutic agents may find use as antibody conjugates.

In an alternate embodiment, the antibody is conjugated or operably linked to a toxin, including but not limited to small molecule toxins and enzymatically active toxins of bacterial, fungal, plant or animal origin, including fragments and/or variants thereof. Small molecule toxins include but are not limited to saporin (Kuroda K, et al., The Prostate 70:1286-1294 (2010); Lip, W L. et al., 2007 Molecular Pharmaceutics 4:241-251; Quadros E V., et al., 2010 Mol Cancer Ther; 9(11); 3033-40; Polito L., et al. 2009 British Journal of Haematology, 147, 710-718), calicheamicin, maytansine (U.S. Pat. No. 5,208,020), trichothene, and CC1065. Toxins include but are not limited to RNase, gelonin, enediynes, ricin, abrin, diptheria toxin, cholera toxin, gelonin, Pseudomonas exotoxin (PE40), Shigella toxin, Clostridium perfringens toxin, and pokeweed antiviral protein.

In one embodiment, an antibody or antigen-binding fragment thereof of the disclosure is conjugated to one or more maytansinoid molecules. Maytansinoids are mitotic inhibitors that act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533. Immunoconjugates containing maytansinoids and their therapeutic use are disclosed, for example, in U.S. Pat. Nos. 5,208,020, 5,416,064 and European Patent EP 0 425 235 B1. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay.

Antibody-maytansinoid conjugates are prepared by chemically linking an antibody to a maytansinoid molecule without significantly diminishing the biological activity of either the antibody or the maytansinoid molecule. An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and non-patent publications referred to hereinabove. Preferred maytansinoids are maytansinol and maytansinol analogues modified in the aromatic ring or at other positions of the maytansinol molecule, such as various maytansinol esters.

Another conjugate of interest comprises an antibody conjugated to one or more calicheamicin molecules. The calicheamicin family of antibiotics is capable of producing double-stranded DNA breaks at sub-picomolar concentrations. Structural analogues of calicheamicin that may also be used (Hinman et al., 1993, Cancer Research 53:3336-3342; Lode et al., 1998, Cancer Research 58:2925-2928) (U.S. Pat. Nos. 5,714,586; 5,712,374; 5,264,586; 5,773,001). Dolastatin 10 analogs such as auristatin E (AE) and monomethylauristatin E (MMAE) may find use as conjugates for the presently disclosed antibodies, or variants thereof (Doronina et al., 2003, Nat Biotechnol 21(7):778-84; Francisco et al., 2003 Blood 102(4):1458-65). Useful enzymatically active toxins include but are not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, for example, PCT WO 93/21232. The present disclosure further contemplates embodiments in which a conjugate or fusion is formed between a VISTA-specific antibody as described herein and a compound with nucleolytic activity, for example a ribonuclease or DNA endonuclease such as a deoxyribonuclease (DNase).

In an alternate embodiment, a herein-disclosed antibody may be conjugated or operably linked to a radioisotope to form a radioconjugate. A variety of radioactive isotopes are available for the production of radioconjugate antibodies. Examples include, but are not limited to ⁹⁰Y, ¹²³I, ¹²⁵I, ¹³¹I, ¹⁸⁶Re, ¹⁸⁸Re, ²¹¹At, and ²¹²Bi.

Antibodies described herein may in certain other embodiments be conjugated to a therapeutic moiety such as a cytotoxin (e.g., a cytostatic or cytocidal agent), a therapeutic agent or a radioactive element (e.g., alpha-emitters, gamma-emitters, etc.). Cytotoxins or cytotoxic agents include any agent that is detrimental to cells. Examples include paclitaxel/paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. One preferred exemplary cytotoxin is saporin (available from Advanced Targeting Systems, San Diego, Calif.). Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC), and anti-mitotic agents (e.g., vincristine and vinblastine).

Moreover, a VISTA-specific antibody (including a functional fragment thereof as provided herein such as an antigen-binding fragment) may in certain embodiments be conjugated to therapeutic moieties such as a radioactive materials or macrocyclic chelators useful for conjugating radiometal ions. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4:2483-90; Peterson et al., 1999, Bioconjug. Chem. 10:553; and Zimmerman et al., 1999, Nucl. Med. Biol. 26:943-50.

In yet another embodiment, an antibody may be conjugated to a “receptor” (such as streptavidin) for utilization in tumor pretargeting wherein the antibody-receptor conjugate is administered to the patient, followed by removal of unbound conjugate from the circulation using a clearing agent and then administration of a “ligand” (e.g. avidin) which is conjugated to a cytotoxic agent (e.g. a radionucleotide). In an alternate embodiment, the antibody is conjugated or operably linked to an enzyme in order to employ Antibody Dependent Enzyme Mediated Prodrug Therapy (ADEPT). ADEPT may be used by conjugating or operably linking the antibody to a prodrug-activating enzyme that converts a prodrug (e.g. a peptidyl chemotherapeutic agent, see PCT WO 81/01145) to an active anti-cancer drug. See, for example, PCT WO 88/07378 and U.S. Pat. No. 4,975,278. The enzyme component of the immunoconjugate useful for ADEPT includes any enzyme capable of acting on a prodrug in such a way so as to convert it into its more active, cytotoxic form. Enzymes that are useful in the method of these and related embodiments include but are not limited to alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as beta-galactosidase and neuramimidase useful for converting glycosylated prodrugs into free drugs; beta-lactamase useful for converting drugs derivatized with beta-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, may be used to convert prodrugs into free active drugs (see, for example, Massey, 1987, Nature 328: 457-458). Antibody-abzyme conjugates can be prepared for delivery of the abzyme to a tumor cell population.

Immunoconjugates may be made using a variety of bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particular coupling agents include N-succinimidyl-3-(2-pyridyldithio)propionate (SPDP) (Carlsson et al., Biochem. J. 173:723-737 [1978]) and N-succinimidyl-4-(2-pyridylthio)pentanoate (SPP) to provide for a disulfide linkage. The linker may be a “cleavable linker” facilitating release of one or more cleavable components. For example, an acid-labile linker may be used (Cancer Research 52: 127-131 (1992); U.S. Pat. No. 5,208,020).

Other modifications of the antibodies (and polypeptides) of the disclosure are also contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol. The antibody also may be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

The desired functional properties of anti-VISTA antibodies may be assessed using a variety of methods known to the skilled person affinity/binding assays (for example, surface plasmon resonance, competitive inhibition assays); cytotoxicity assays, cell viability assays, cell proliferation or differentiation assays, cancer cell and/or tumor growth inhibition using in vitro or in vivo models. Other assays may test the ability of antibodies described herein to block normal VISTA-mediated responses, such as inhibition of T cell proliferation, inhibition of T cell activation, inhibition of T cell cytokine production (e.g., IFNγ and IL-2), and inducing IL-6, IL-8, IL-1 beta, and TNF-alpha production by monocytes. The antibodies described herein may also be tested for in vitro and in vivo efficacy. Such assays may be performed using well-established protocols known to the skilled person (see e.g., Current Protocols in Molecular Biology (Greene Publ. Assoc. Inc. & John Wiley & Sons, Inc., NY, NY); Current Protocols in Immunology (Edited by: John E. Coligan, Ada M. Kruisbeek, David H. Margulies, Ethan M. Shevach, Warren Strober 2001 John Wiley & Sons, NY, NY); or commercially available kits.

The present disclosure further provides in certain embodiments an isolated nucleic acid encoding an antibody or antigen-binding fragment thereof as described herein, for instance, a nucleic acid which codes for a CDR or VH or VL domain as described herein. Nucleic acids include DNA and RNA. These and related embodiments may include polynucleotides encoding antibodies that bind VISTA as described herein. The term “isolated polynucleotide” as used herein shall mean a polynucleotide of genomic, cDNA, or synthetic origin or some combination thereof, which by virtue of its origin the isolated polynucleotide (1) is not associated with all or a portion of a polynucleotide in which the isolated polynucleotide is found in nature, (2) is linked to a polynucleotide to which it is not linked in nature, or (3) does not occur in nature as part of a larger sequence.

The term “operably linked” means that the components to which the term is applied are in a relationship that allows them to carry out their inherent functions under suitable conditions. For example, a transcription control sequence “operably linked” to a protein coding sequence is ligated thereto so that expression of the protein coding sequence is achieved under conditions compatible with the transcriptional activity of the control sequences.

The term “control sequence” as used herein refers to polynucleotide sequences that can affect expression, processing or intracellular localization of coding sequences to which they are ligated or operably linked. The nature of such control sequences may depend upon the host organism. In particular embodiments, transcription control sequences for prokaryotes may include a promoter, ribosomal binding site, and transcription termination sequence. In other particular embodiments, transcription control sequences for eukaryotes may include promoters comprising one or a plurality of recognition sites for transcription factors, transcription enhancer sequences, transcription termination sequences and polyadenylation sequences. In certain embodiments, “control sequences” can include leader sequences and/or fusion partner sequences.

The term “polynucleotide” as referred to herein means single-stranded or double-stranded nucleic acid polymers. In certain embodiments, the nucleotides comprising the polynucleotide can be ribonucleotides or deoxyribonucleotides or a modified form of either type of nucleotide. Said modifications include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al., 1986, Nucl. Acids Res., 14:9081; Stec et al., 1984, J. Am. Chem. Soc., 106:6077; Stein et al., 1988, Nucl. Acids Res., 16:3209; Zon et al., 1991, Anti-Cancer Drug Design, 6:539; Zon et al., 1991, OLIGONUCLEOTIDES AND ANALOGUES: A PRACTICAL APPROACH, pp. 87-108 (F. Eckstein, Ed.), Oxford University Press, Oxford England; Stec et al., U.S. Pat. No. 5,151,510; Uhlmann and Peyman, 1990, Chemical Reviews, 90:543, the disclosures of which are hereby incorporated by reference for any purpose. An oligonucleotide can include a detectable label to enable detection of the oligonucleotide or hybridization thereof.

The term “vector” is used to refer to any molecule (e.g., nucleic acid, plasmid, or virus) used to transfer coding information to a host cell. The term “expression vector” refers to a vector that is suitable for transformation of a host cell and contains nucleic acid sequences that direct and/or control expression of inserted heterologous nucleic acid sequences. Expression includes, but is not limited to, processes such as transcription, translation, and RNA splicing, if introns are present.

As will be understood by those skilled in the art, polynucleotides may include genomic sequences, extra-genomic and plasmid-encoded sequences and smaller engineered gene segments that express, or may be adapted to express, proteins, polypeptides, peptides and the like. Such segments may be naturally isolated, or modified synthetically by the skilled person.

As will be also recognized by the skilled artisan, polynucleotides may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide according to the present disclosure, and a polynucleotide may, but need not, be linked to other molecules and/or support materials. Polynucleotides may comprise a native sequence or may comprise a sequence that encodes a variant or derivative of such a sequence.

Therefore, according to these and related embodiments, the present disclosure also provides polynucleotides encoding the anti-VISTA antibodies described herein. In certain embodiments, polynucleotides are provided that comprise some or all of a polynucleotide sequence encoding an antibody as described herein and complements of such polynucleotides.

In other related embodiments, polynucleotide variants may have substantial identity to a polynucleotide sequence encoding an anti-VISTA antibody described herein. For example, a polynucleotide may be a polynucleotide comprising at least 70% sequence identity, preferably at least 75%, 80%, 85%, 90%, 95%, 96%, 97%, 98%, or 99% or higher, sequence identity compared to a reference polynucleotide sequence such as a sequence encoding an antibody described herein, using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the binding affinity of the antibody encoded by the variant polynucleotide is not substantially diminished relative to an antibody encoded by a polynucleotide sequence specifically set forth herein.

In certain other related embodiments, polynucleotide fragments may comprise or consist essentially of various lengths of contiguous stretches of sequence identical to or complementary to a sequence encoding an antibody as described herein. For example, polynucleotides are provided that comprise or consist essentially of at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, 150, 200, 300, 400, 500 or 1000 or more contiguous nucleotides of a sequences the encodes an antibody, or antigen-binding fragment thereof, disclosed herein as well as all intermediate lengths there between. It will be readily understood that “intermediate lengths”, in this context, means any length between the quoted values, such as 50, 51, 52, 53, etc.; 100, 101, 102, 103, etc.; 150, 151, 152, 153, etc.; including all integers through 200-500; 500-1,000, and the like. A polynucleotide sequence as described here may be extended at one or both ends by additional nucleotides not found in the native sequence. This additional sequence may consist of 1, 2, 3, 4, 5, 6, 7, 8, 9, 10, 11, 12, 13, 14, 15, 16, 17, 18, 19, or 20 nucleotides at either end of a polynucleotide encoding an antibody described herein or at both ends of a polynucleotide encoding an antibody described herein.

In another embodiment, polynucleotides are provided that are capable of hybridizing under moderate to high stringency conditions to a polynucleotide sequence encoding an antibody, or antigen-binding fragment thereof, provided herein, or a fragment thereof, or a complementary sequence thereof. Hybridization techniques are well known in the art of molecular biology. For purposes of illustration, suitable moderately stringent conditions for testing the hybridization of a polynucleotide as provided herein with other polynucleotides include prewashing in a solution of 5×SSC, 0.5% SDS, 1.0 mM EDTA (pH 8.0); hybridizing at 50° C.-60° C., 5×SSC, overnight; followed by washing twice at 65° C. for 20 minutes with each of 2×, 0.5× and 0.2×SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60° C.-65° C. or 65° C.-70° C.

In certain embodiments, the polynucleotides described above, e.g., polynucleotide variants, fragments and hybridizing sequences, encode antibodies that bind VISTA, or antigen-binding fragments thereof. In other embodiments, such polynucleotides encode antibodies or antigen-binding fragments, or CDRs thereof, that bind to VISTA at least about 50%, at least about 70%, and in certain embodiments, at least about 90% as well as an antibody sequence specifically set forth herein. In further embodiments, such polynucleotides encode antibodies or antigen-binding fragments, or CDRs thereof, that bind to VISTA with greater affinity than the antibodies set forth herein, for example, that bind quantitatively at least about 105%, 106%, 107%, 108%, 109%, or 110% as well as an antibody sequence specifically set forth herein.

As described elsewhere herein, determination of the three-dimensional structures of representative polypeptides (e.g., variant VISTA-specific antibodies as provided herein, for instance, an antibody protein having an antigen-binding fragment as provided herein) may be made through routine methodologies such that substitution, addition, deletion or insertion of one or more amino acids with selected natural or non-natural amino acids can be virtually modeled for purposes of determining whether a so derived structural variant retains the space-filling properties of presently disclosed species. A variety of computer programs are known to the skilled artisan for determining appropriate amino acid substitutions (or appropriate polynucleotides encoding the amino acid sequence) within an antibody such that, for example, affinity is maintained or better affinity is achieved.

The polynucleotides described herein, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign™ program in the Lasergene® suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., Unified Approach to Alignment and Phylogenes, pp. 626-645 (1990); Methods in Enzymology vol. 183, Academic Press, Inc., San Diego, Calif.; Higgins, D. G. and Sharp, P. M., CABIOS 5:151-153 (1989); Myers, E. W. and Muller W., CABIOS 4:11-17 (1988); Robinson, E. D., Comb. Theor 11:105 (1971); Santou, N. Nes, M., Mol. Biol. Evol. 4:406-425 (1987); Sneath, P. H. A. and Sokal, R. R., Numerical Taxonomy—the Principles and Practice of Numerical Taxonomy, Freeman Press, San Francisco, Calif. (1973); Wilbur, W. J. and Lipman, D. J., Proc. Natl. Acad., Sci. USA 80:726-730 (1983).

Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection. One preferred example of algorithms that are suitable for determining percent sequence identity and sequence similarity are the BLAST and BLAST 2.0 algorithms, which are described in Altschul et al., Nucl. Acids Res. 25:3389-3402 (1977), and Altschul et al., J. Mol. Biol. 215:403-410 (1990), respectively. BLAST and BLAST 2.0 can be used, for example with the parameters described herein, to determine percent sequence identity among two or more the polynucleotides. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a word length (W) of 11, and expectation (E) of 10, and the BLOSUM62 scoring matrix (see Henikoff and Henikoff, Proc. Natl. Acad. Sci. USA 89:10915 (1989)) alignments, (B) of 50, expectation (E) of 10, M=5, N=−4 and a comparison of both strands.

In certain embodiments, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encodes an antibody as described herein. Some of these polynucleotides bear minimal sequence identity to the nucleotide sequence of the native or original polynucleotide sequence that encode antibodies that bind to VISTA. Nonetheless, polynucleotides that vary due to differences in codon usage are expressly contemplated by the present disclosure. In certain embodiments, sequences that have been codon-optimized for mammalian expression are specifically contemplated.

Therefore, in another embodiment, a mutagenesis approach, such as site-specific mutagenesis, may be employed for the preparation of variants and/or derivatives of the antibodies described herein. By this approach, specific modifications in a polypeptide sequence can be made through mutagenesis of the underlying polynucleotides that encode them. These techniques provides a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments, the inventors contemplate the mutagenesis of the polynucleotide sequences that encode an antibody disclosed herein, or an antigen-binding fragment thereof, to alter one or more properties of the encoded polypeptide, such as the binding affinity of the antibody or the antigen-binding fragment thereof, or the function of a particular Fc region, or the affinity of the Fc region for a particular FcγR. The techniques of site-specific mutagenesis are well-known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In another approach for the production of polypeptide variants, recursive sequence recombination, as described in U.S. Pat. No. 5,837,458, may be employed. In this approach, iterative cycles of recombination and screening or selection are performed to “evolve” individual polynucleotide variants having, for example, increased binding affinity. Certain embodiments also provide constructs in the form of plasmids, vectors, transcription or expression cassettes which comprise at least one polynucleotide as described herein.

In many embodiments, the nucleic acids encoding a subject monoclonal antibody are introduced directly into a host cell, and the cell incubated under conditions sufficient to induce expression of the encoded antibody. The antibodies of this disclosure are prepared using standard techniques well known to those of skill in the art in combination with the polypeptide and nucleic acid sequences provided herein. The polypeptide sequences may be used to determine appropriate nucleic acid sequences encoding the particular antibody disclosed thereby. The nucleic acid sequence may be optimized to reflect particular codon “preferences” for various expression systems according to standard methods well known to those of skill in the art.

According to certain related embodiments there is provided a recombinant host cell which comprises one or more constructs as described herein; a nucleic acid encoding any antibody, CDR, VH or VL domain, or antigen-binding fragment thereof; and a method of production of the encoded product, which method comprises expression from encoding nucleic acid therefor. Expression may conveniently be achieved by culturing under appropriate conditions recombinant host cells containing the nucleic acid. Following production by expression, an antibody or antigen-binding fragment thereof, may be isolated and/or purified using any suitable technique, and then used as desired.

Antibodies or antigen-binding fragments thereof as provided herein, and encoding nucleic acid molecules and vectors, may be isolated and/or purified, e.g. from their natural environment, in substantially pure or homogeneous form, or, in the case of nucleic acid, free or substantially free of nucleic acid or genes of origin other than the sequence encoding a polypeptide with the desired function. Nucleic acid may comprise DNA or RNA and may be wholly or partially synthetic. Reference to a nucleotide sequence as set out herein encompasses a DNA molecule with the specified sequence, and encompasses a RNA molecule with the specified sequence in which U is substituted for T, unless context requires otherwise.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli.

The expression of antibodies and antigen-binding fragments in prokaryotic cells such as E. coli is well established in the art. For a review, see for example Pluckthun, A. Bio/Technology 9: 545-551 (1991). Expression in eukaryotic cells in culture is also available to those skilled in the art as an option for production of antibodies or antigen-binding fragments thereof, see recent reviews, for example Ref, M. E. (1993) Curr. Opinion Biotech. 4: 573-576; Trill J. J. et al. (1995) Curr. Opinion Biotech 6: 553-560.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992, or subsequent updates thereto.

The term “host cell” is used to refer to a cell into which has been introduced, or which is capable of having introduced into it, a nucleic acid sequence encoding one or more of the herein described antibodies, and which further expresses or is capable of expressing a selected gene of interest, such as a gene encoding any herein described antibody. The term includes the progeny of the parent cell, whether or not the progeny are identical in morphology or in genetic make-up to the original parent, so long as the selected gene is present. Accordingly there is also contemplated a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g. by culturing host cells under conditions for expression of the gene. In one embodiment, the nucleic acid is integrated into the genome (e.g. chromosome) of the host cell. Integration may be promoted by inclusion of sequences which promote recombination with the genome, in accordance-with standard techniques.

The present disclosure also provides, in certain embodiments, a method which comprises using a construct as stated above in an expression system in order to express a particular polypeptide such as a VISTA-specific antibody as described herein.

The term “transduction” is used to refer to the transfer of genes from one bacterium to another, usually by a phage. “Transduction” also refers to the acquisition and transfer of eukaryotic cellular sequences by retroviruses. The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, MOLECULAR CLONING, A LABORATORY MANUAL, Cold Spring Harbor Laboratories; Davis et al., 1986, BASIC METHODS IN MOLECULAR BIOLOGY, Elsevier; and Chu et al., 1981, Gene 13:197. Such techniques can be used to introduce one or more exogenous DNA moieties into suitable host cells.

The term “transformation” as used herein refers to a change in a cell's genetic characteristics, and a cell has been transformed when it has been modified to contain a new DNA. For example, a cell is transformed where it is genetically modified from its native state. Following transfection or transduction, the transforming DNA may recombine with that of the cell by physically integrating into a chromosome of the cell, or may be maintained transiently as an episomal element without being replicated, or may replicate independently as a plasmid. A cell is considered to have been stably transformed when the DNA is replicated with the division of the cell. The term “naturally occurring” or “native” when used in connection with biological materials such as nucleic acid molecules, polypeptides, host cells, and the like, refers to materials which are found in nature and are not manipulated by a human. Similarly, “non-naturally occurring” or “non-native” as used herein refers to a material that is not found in nature or that has been structurally modified or synthesized by a human.

The terms “polypeptide” “protein” and “peptide” and “glycoprotein” are used interchangeably and mean a polymer of amino acids not limited to any particular length. The term does not exclude modifications such as myristoylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. The terms “polypeptide” and “protein” specifically encompass the antibodies that bind to VISTA of the present disclosure, or sequences that have deletions from, additions to, and/or substitutions of one or more amino acid of an anti-VISTA antibody. Thus, a “polypeptide” or a “protein” can comprise one (termed “a monomer”) or a plurality (termed “a multimer”) of amino acid chains.

The term “isolated protein” referred to herein means that a subject protein (1) is free of at least some other proteins with which it would typically be found in nature, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or noncovalent interaction) with portions of a protein with which the “isolated protein” is associated in nature, (6) is operably associated (by covalent or noncovalent interaction) with a polypeptide with which it is not associated in nature, or (7) does not occur in nature. Such an isolated protein can be encoded by genomic DNA, cDNA, mRNA or other RNA, of may be of synthetic origin, or any combination thereof. In certain embodiments, the isolated protein is substantially free from proteins or polypeptides or other contaminants that are found in its natural environment that would interfere with its use (therapeutic, diagnostic, prophylactic, research or otherwise).

The term “polypeptide fragment” refers to a polypeptide, which can be monomeric or multimeric, that has an amino-terminal deletion, a carboxyl-terminal deletion, and/or an internal deletion or substitution of a naturally-occurring or recombinantly-produced polypeptide. In certain embodiments, a polypeptide fragment can comprise an amino acid chain at least 5 to about 500 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, or 450 amino acids long. Particularly useful polypeptide fragments include functional domains, including antigen-binding domains or fragments of antibodies. In the case of an anti-VISTA antibody, useful fragments include, but are not limited to: a CDR region, especially a CDR3 region of the heavy or light chain; a variable region of a heavy or light chain; a portion of an antibody chain or just its variable region including two CDRs; and the like.

Polypeptides may comprise a signal (or leader) sequence at the N-terminal end of the protein, which co-translationally or post-translationally directs transfer of the protein. Any polypeptide amino acid sequences provided herein that include a signal peptide are also contemplated for any use described herein without such a signal or leader peptide. As would be recognized by the skilled person, the signal peptide is usually cleaved during processing and is not included in the active antibody protein. The polypeptide may also be fused in-frame or conjugated to a linker or other sequence for ease of synthesis, purification or identification of the polypeptide (e.g., poly-His), or to enhance binding of the polypeptide to a solid support.

A peptide linker/spacer sequence may also be employed to separate multiple polypeptide components by a distance sufficient to ensure that each polypeptide folds into its secondary and/or tertiary structures, if desired. Such a peptide linker sequence can be incorporated into a fusion polypeptide using standard techniques well known in the art.

Certain peptide spacer sequences may be chosen, for example, based on: (1) their ability to adopt a flexible extended conformation; (2) their inability to adopt a secondary structure that could interact with functional epitopes on the first and second polypeptides; and/or (3) the lack of hydrophobic or charged residues that might react with the polypeptide functional epitopes.

In one illustrative embodiment, peptide spacer sequences contain, for example, Gly, Asn and Ser residues. Other near neutral amino acids, such as Thr and Ala, may also be included in the spacer sequence.

Other amino acid sequences which may be usefully employed as spacers include those disclosed in Maratea et al., Gene 40:39 46 (1985); Murphy et al., Proc. Natl. Acad. Sci. USA 83:8258 8262 (1986); U.S. Pat. Nos. 4,935,233 and 4,751,180.

Other illustrative spacers may include, for example, Glu-Gly-Lys-Ser-Ser-Gly-Ser-Gly-Ser-Glu-Ser-Lys-Val-Asp (SEQ ID NO:X) (Chaudhary et al., 1990, Proc. Natl. Acad. Sci. U.S.A. 87:1066-1070) and Lys-Glu-Ser-Gly-Ser-Val-Ser-Ser-Glu-Gln-Leu-Ala-Gln-Phe-Arg-Ser-Leu-Asp (SEQ ID NO:X) (Bird et al., 1988, Science 242:423-426).

In some embodiments, spacer sequences are not required when the first and second polypeptides have non-essential N-terminal amino acid regions that can be used to separate the functional domains and prevent steric interference. Two coding sequences can be fused directly without any spacer or by using a flexible polylinker composed, for example, of the pentamer Gly-Gly-Gly-Gly-Ser (SEQ ID NO:X) repeated 1 to 3 times. Such a spacer has been used in constructing single chain antibodies (scFv) by being inserted between VH and VL (Bird et al., 1988, Science 242:423-426; Huston et al., 1988, Proc. Natl. Acad. Sci. U.S.A. 85:5979-5883).

A peptide spacer, in certain embodiments, is designed to enable the correct interaction between two beta-sheets forming the variable region of the single chain antibody.

In certain illustrative embodiments, a peptide spacer is between 1 to 5 amino acids, between 5 to 10 amino acids, between 5 to 25 amino acids, between 5 to 50 amino acids, between 10 to 25 amino acids, between 10 to 50 amino acids, between 10 to 100 amino acids, or any intervening range of amino acids.

In other illustrative embodiments, a peptide spacer comprises about 1, 5, 10, 15, 20, 25, 30, 35, 40, 45, 50 or more amino acids in length.

Amino acid sequence modification(s) of the antibodies described herein are contemplated. For example, it may be desirable to improve the binding affinity and/or other biological properties of the antibody. For example, amino acid sequence variants of an antibody may be prepared by introducing appropriate nucleotide changes into a polynucleotide that encodes the antibody, or a chain thereof, or by peptide synthesis. Such modifications include, for example, deletions from, and/or insertions into and/or substitutions of, residues within the amino acid sequences of the antibody. Any combination of deletion, insertion, and substitution may be made to arrive at the final antibody, provided that the final construct possesses the desired characteristics (e.g., high affinity binding to VISTA). The amino acid changes also may alter post-translational processes of the antibody, such as changing the number or position of glycosylation sites. Any of the variations and modifications described above for polypeptides of the present disclosure may be included in antibodies of the present disclosure.

The present disclosure provides variants of the antibodies disclosed herein. In certain embodiments, such variant antibodies or antigen-binding fragments, or CDRs thereof, bind to VISTA at least about 50%, at least about 70%, and in certain embodiments, at least about 90% as well as an antibody sequence specifically set forth herein. In further embodiments, such variant antibodies or antigen-binding fragments, or CDRs thereof, bind to VISTA with greater affinity than the antibodies set forth herein, for example, that bind quantitatively at least about 105%, 106%, 107%, 108%, 109%, or 110% as well as an antibody sequence specifically set forth herein.

In particular embodiments, a subject antibody may have: a) a heavy chain variable region having an amino acid sequence that is at least 80% identical, at least 95% identical, at least 90%, at least 95% or at least 98% or 99% identical, to the heavy chain variable region of an anti-VISTA antibody described herein; and b) a light chain variable region having an amino acid sequence that is at least 80% identical, at least 85%, at least 90%, at least 95% or at least 98% or 99% identical, to the light chain variable region of an anti-VISTA antibody described herein. The amino acid sequence of illustrative heavy and light chain regions are set forth in SEQ ID NOs:1-113.

In particular embodiments, the antibody may comprise: a) a heavy chain variable region comprising: i. a CDR1 region that is identical in amino acid sequence to the heavy chain CDR1 region of a selected antibody described herein; ii. a CDR2 region that is identical in amino acid sequence to the heavy chain CDR2 region of the selected antibody; and iii. a CDR3 region that is identical in amino acid sequence to the heavy chain CDR3 region of the selected antibody; and b) a light chain variable domain comprising: i. a CDR1 region that is identical in amino acid sequence to the light chain CDR1 region of the selected antibody; ii. a CDR2 region that is identical in amino acid sequence to the light chain CDR2 region of the selected antibody; and iii. a CDR3 region that is identical in amino acid sequence to the light chain CDR3 region of the selected antibody; wherein the antibody specifically binds a selected target (e.g., VISTA). In a further embodiment, the antibody, or antigen-binding fragment thereof, is a variant antibody wherein the variant comprises a heavy and light chain identical to the selected antibody except for up to 8, 9, 10, 11, 12, 13, 14, 15, or more amino acid substitutions in the CDR regions of the VH and VL regions. In this regard, there may be 1, 2, 3, 4, 5, 6, 7, 8, or in certain embodiments, 9, 10, 11, 12, 13, 14, 15 more amino acid substitutions in the CDR regions of the selected antibody. Substitutions may be in CDRs either in the VH and/or the VL regions. (See e.g., Muller, 1998, Structure 6:1153-1167).

Determination of the three-dimensional structures of representative polypeptides (e.g., variant VISTA-specific antibodies as provided herein, for instance, an antibody protein having an antigen-binding fragment as provided herein) may be made through routine methodologies such that substitution, addition, deletion or insertion of one or more amino acids with selected natural or non-natural amino acids can be virtually modeled for purposes of determining whether a so derived structural variant retains the space-filling properties of presently disclosed species. See, for instance, Donate et al., 1994 Prot. Sci. 3:2378; Bradley et al., Science 309: 1868-1871 (2005); Schueler-Furman et al., Science 310:638 (2005); Dietz et al., Proc. Nat. Acad. Sci. USA 103:1244 (2006); Dodson et al., Nature 450:176 (2007); Qian et al., Nature 450:259 (2007); Raman et al. Science 327:1014-1018 (2010). Some additional non-limiting examples of computer algorithms that may be used for these and related embodiments, such as for rational design of VISTA-specific antibodies antigen-binding domains thereof as provided herein, include VMD which is a molecular visualization program for displaying, animating, and analyzing large biomolecular systems using 3-D graphics and built-in scripting (see the website for the Theoretical and Computational Biophysics Group, University of Illinois at Urbana-Champagne, at ks.uiuc.edu/Research/vmd/. Many other computer programs are known in the art and available to the skilled person and which allow for determining atomic dimensions from space-filling models (van der Waals radii) of energy-minimized conformations; GRID, which seeks to determine regions of high affinity for different chemical groups, thereby enhancing binding, Monte Carlo searches, which calculate mathematical alignment, and CHARMM (Brooks et al. (1983) J. Comput. Chem. 4:187-217) and AMBER (Weiner et al (1981) J. Comput Chem. 106: 765), which assess force field calculations, and analysis (see also, Eisenfield et al. (1991) Am. J. Physiol. 261:C376-386; Lybrand (1991) J. Pharm. Belg. 46:49-54; Froimowitz (1990) Biotechniques 8:640-644; Burbam et al. (1990) Proteins 7:99-111; Pedersen (1985) Environ. Health Perspect. 61:185-190; and Kini et al. (1991) J. Biomol. Struct Dyn. 9:475-488). A variety of appropriate computational computer programs are also commercially available, such as from Schrödinger (Munich, Germany).

In another embodiment, the anti-VISTA antibodies and humanized versions thereof are derived from rabbit monoclonal antibodies, and in particular are generated using RabMAb® technology. These antibodies are advantageous as they require minimal sequence modifications, thereby facilitating retention of functional properties after humanization using mutational lineage guided (MLG) humanization technology (see e.g., U.S. Pat. No. 7,462,697). Thus, illustrative methods for making the anti-VISTA antibodies of the present disclosure include the RabMab® rabbit monoclonal antibody technology described, for example, in U.S. Pat. Nos. 5,675,063 and 7,429,487. In this regard, in certain embodiments, the anti-VISTA antibodies of the disclosure are produced in rabbits. In particular embodiments, a rabbit-derived immortal B-lymphocyte capable of fusion with a rabbit splenocyte or peripheral B lymphocyte is used to produce a hybrid cell that produces an antibody. The immortal B-lymphocyte does not detectably express endogenous immunoglobulin heavy chain and may contain, in certain embodiments, an altered immunoglobulin heavy chain-encoding gene.

### Compositions and Methods of Use

The present disclosure provides compositions comprising the VISTA-specific antibodies, or antigen-binding fragments thereof, and administration of such composition in a variety of therapeutic settings, including the treatment of cancers, inflammatory diseases, and infectious diseases.

Administration of the VISTA-specific antibodies described herein, in pure form or in an appropriate pharmaceutical composition, can be carried out via any of the accepted modes of administration of agents for serving similar utilities. The pharmaceutical compositions can be prepared by combining an antibody or antibody-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other anti-cancer agents as described elsewhere herein) and/or suitable excipients such as salts, buffers and stabilizers may, but need not, be present within the composition. Administration may be achieved by a variety of different routes, including oral, parenteral, nasal, intravenous, intradermal, subcutaneous or topical. Preferred modes of administration depend upon the nature of the condition to be treated or prevented. An amount that, following administration, reduces, inhibits, prevents or delays the progression and/or metastasis of a cancer is considered effective.

In certain embodiments, the amount administered is sufficient to result in tumor regression, as indicated by a statistically significant decrease in the amount of viable tumor, for example, at least a 50% decrease in tumor mass, or by altered (e.g., decreased with statistical significance) scan dimensions.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

The VISTA-specific antibody-containing compositions may be administered alone or in combination with other known cancer treatments, such as radiation therapy, chemotherapy, transplantation, immunotherapy, hormone therapy, photodynamic therapy, etc. The compositions may also be administered in combination with antibiotics.

Typical routes of administering these and related pharmaceutical compositions thus include, without limitation, oral, topical, transdermal, inhalation, parenteral, sublingual, buccal, rectal, vaginal, intravitreal, and intranasal. The term parenteral as used herein includes subcutaneous injections, intravenous, intramuscular, intrasternal injection or infusion techniques. Pharmaceutical compositions according to certain embodiments are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described VISTA-specific antibody in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of an antibody of the present disclosure, for treatment of a disease or condition of interest in accordance with teachings herein.

A pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition intended for either parenteral or oral administration should contain an amount of a VISTA-specific antibody as herein disclosed such that a suitable dosage will be obtained. Typically, this amount is at least 0.01% of the antibody in the composition. When intended for oral administration, this amount may be varied to be between 0.1 and about 70% of the weight of the composition. Certain oral pharmaceutical compositions contain between about 4% and about 75% of the antibody. In certain embodiments, pharmaceutical compositions and preparations are prepared so that a parenteral dosage unit contains between 0.01 to 10% by weight of the antibody prior to dilution.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The pharmaceutical composition in solid or liquid form may include an agent that binds to the antibody and thereby assists in the delivery of the compound. Suitable agents that may act in this capacity include other monoclonal or polyclonal antibodies, one or more proteins or a liposome. The pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The pharmaceutical compositions may be prepared by methodology well known in the pharmaceutical art. For example, a pharmaceutical composition intended to be administered by injection can be prepared by combining a composition that comprises a VISTA-specific antibody as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the antibody composition so as to facilitate dissolution or homogeneous suspension of the antibody in the aqueous delivery system.

The compositions may be administered in a therapeutically effective amount, which will vary depending upon a variety of factors including the activity of the specific compound (e.g., VISTA-specific antibody) employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g).

Compositions comprising the VISTA-specific antibodies of the present disclosure may also be administered simultaneously with, prior to, or after administration of one or more other therapeutic agents. Such combination therapy may include administration of a single pharmaceutical dosage formulation which contains an antibody and one or more additional active agents, as well as administration of compositions comprising antibodies of the disclosure and each active agent in its own separate pharmaceutical dosage formulation. For example, an antibody as described herein and the other active agent can be administered to the patient together in a single oral dosage composition such as a tablet or capsule, or each agent administered in separate oral dosage formulations. Similarly, an antibody as described herein and the other active agent can be administered to the patient together in a single parenteral dosage composition such as in a saline solution or other physiologically acceptable solution, or each agent administered in separate parenteral dosage formulations. Where separate dosage formulations are used, the compositions comprising antibodies and one or more additional active agents can be administered at essentially the same time, i.e., concurrently, or at separately staggered times, i.e., sequentially and in any order; combination therapy is understood to include all these regimens.

Thus, in certain embodiments, also contemplated is the administration of anti-VISTA antibody compositions of this disclosure in combination with one or more other therapeutic agents. Such therapeutic agents may be accepted in the art as a standard treatment for a particular disease state as described herein, such as rheumatoid arthritis, inflammation or cancer. Exemplary therapeutic agents contemplated include cytokines, growth factors, steroids, NSAIDs, DMARDs, anti-inflammatories, chemotherapeutics, radiotherapeutics, or other active and ancillary agents.

In certain embodiments, the anti-VISTA antibodies disclosed herein are administered in combination with one or more cancer immunotherapy agents. In certain instances, an immunotherapy agent modulates the immune response of a subject, for example, to increase or maintain a cancer-related or cancer-specific immune response, and thereby results in increased immune cell inhibition or reduction of cancer cells. Exemplary immunotherapy agents include polypeptides, for example, antibodies and antigen-binding fragments thereof, ligands, and small peptides, and mixtures thereof. Also include as immunotherapy agents are small molecules, cells (e.g., immune cells such as T-cells), various cancer vaccines, gene therapy or other polynucleotide-based agents, including viral agents such as oncolytic viruses, and others known in the art. Thus, in certain embodiments, the cancer immunotherapy agent is selected from one or more of immune checkpoint modulatory agents, cancer vaccines, oncolytic viruses, cytokines, and a cell-based immunotherapies.

In certain embodiments, the cancer immunotherapy agent is an immune checkpoint modulatory agent. Particular examples include “antagonists” of one or more inhibitory immune checkpoint molecules, and “agonists” of one or more stimulatory immune checkpoint molecules. Generally, immune checkpoint molecules are components of the immune system that either turn up a signal (co-stimulatory molecules) or turn down a signal, the targeting of which has therapeutic potential in cancer because cancer cells can perturb the natural function of immune checkpoint molecules (see, e.g., Sharma and Allison, Science. 348:56-61, 2015; Topalian et al., Cancer Cell. 27:450-461, 2015; Pardoll, Nature Reviews Cancer. 12:252-264, 2012). In some embodiments, the immune checkpoint modulatory agent (e.g., antagonist, agonist) “binds” or “specifically binds” to the one or more immune checkpoint molecules, as described herein.

In some embodiments, the immune checkpoint modulatory agent is an antagonist or inhibitor of one or more inhibitory immune checkpoint molecules. Exemplary inhibitory immune checkpoint molecules include Programmed Death-Ligand 1 (PD-L1), Programmed Death-Ligand 2 (PD-L2), Programmed Death 1 (PD-1), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), B and T Lymphocyte Attenuator (BTLA), CD160, and T-cell immunoreceptor with Ig and ITIM domains (TIGIT).

In certain embodiments, the agent is a PD-1 (receptor) antagonist or inhibitor, the targeting of which has been shown to restore immune function in the tumor environment (see, e.g., Phillips et al., Int Immunol. 27:39-46, 2015). PD-1 is a cell surface receptor that belongs to the immunoglobulin superfamily and is expressed on T cells and pro-B cells. PD-1 interacts with two ligands, PD-L1 and PD-L2. PD-1 functions as an inhibitory immune checkpoint molecule, for example, by reducing or preventing the activation of T-cells, which in turn reduces autoimmunity and promotes self-tolerance. The inhibitory effect of PD-1 is accomplished at least in part through a dual mechanism of promoting apoptosis in antigen specific T-cells in lymph nodes while also reducing apoptosis in regulatory T cells (suppressor T cells). Some examples of PD-1 antagonists or inhibitors include an antibody or antigen-binding fragment or small molecule that specifically binds to PD-1 and reduces one or more of its immune-suppressive activities, for example, its downstream signaling or its interaction with PD-L1. Specific examples of PD-1 antagonists or inhibitors include the antibodies nivolumab, pembrolizumab, PDR001, MK-3475, AMP-224, AMP-514, and pidilizumab, and antigen-binding fragments thereof (see, e.g., U.S. Pat. Nos. 8,008,449; 8,993,731; 9,073,994; 9,084,776; 9,102,727; 9,102,728; 9,181,342; 9,217,034; 9,387,247; 9,492,539; 9,492,540; and U.S. Application Nos. 2012/0039906; 2015/0203579).

In some embodiments, the agent is a PD-L1 antagonist or inhibitor. As noted above, PD-L1 is one of the natural ligands for the PD-1 receptor. General examples of PD-L1 antagonists or inhibitors include an antibody or antigen-binding fragment or small molecule that specifically binds to PD-L1 and reduces one or more of its immune-suppressive activities, for example, its binding to the PD-1 receptor. Specific examples of PD-L1 antagonists include the antibodies atezolizumab (MPDL3280A), avelumab (MSB0010718C), and durvalumab (MED14736), and antigen-binding fragments thereof (see, e.g., U.S. Pat. Nos. 9,102,725; 9,393,301; 9,402,899; 9,439,962).

In some embodiments, the agent is a PD-L2 antagonist or inhibitor. As noted above, PD-L2 is one of the natural ligands for the PD-1 receptor. General examples of PD-L2 antagonists or inhibitors include an antibody or antigen-binding fragment or small molecule that specifically binds to PD-L2 and reduces one or more of its immune-suppressive activities, for example, its binding to the PD-1 receptor.

In some embodiments, the agent is a CTLA-4 antagonist or inhibitor. CTLA4 or CTLA-4 (cytotoxic T-lymphocyte-associated protein 4), also known as CD152 (cluster of differentiation 152), is a protein receptor that functions as an inhibitory immune checkpoint molecule, for example, by transmitting inhibitory signals to T-cells when it is bound to CD80 or CD86 on the surface of antigen-presenting cells. General examples CTLA-4 antagonists or inhibitors include an antibody or antigen-binding fragment or small molecule that specifically binds to CTLA-4. Particular examples include the antibodies ipilimumab and tremelimumab, and antigen-binding fragments thereof. At least some of the activity of ipilimumab is believed to be mediated by antibody-dependent cell-mediated cytotoxicity (ADCC) killing of suppressor Tregs that express CTLA-4.

In some embodiments, the agent is an IDO antagonist or inhibitor, or a TDO antagonist or inhibitor. IDO and TDO are tryptophan catabolic enzymes with immune-inhibitory properties. For example, IDO is known to suppress T-cells and NK cells, generate and activate Tregs and myeloid-derived suppressor cells, and promote tumor angiogenesis. General examples of IDO and TDO antagonists or inhibitors include an antibody or antigen-binding fragment or small molecule that specifically binds to IDO or TDO (see, e.g., Platten et al., Front Immunol. 5: 673, 2014) and reduces or inhibits one or more immune-suppressive activities. Specific examples of IDO antagonists or inhibitors include indoximod (NLG-8189), 1-methyl-tryptophan (1 MT), β-Carboline (norharmane; 9H-pyrido[3,4-b]indole), rosmarinic acid, and epacadostat (see, e.g., Sheridan, Nature Biotechnology. 33:321-322, 2015). Specific examples of TDO antagonists or inhibitors include 680C91 and LM10 (see, e.g., Pilotte et al., PNAS USA. 109:2497-2502, 2012).

In some embodiments, the agent is a TIM-3 antagonist or inhibitor. T-cell Immunoglobulin domain and Mucin domain 3 (TIM-3) is expressed on activated human CD4+ T-cells and regulates Th1 and Th17 cytokines. TIM-3 also acts as a negative regulator of Th1/Tc1 function by triggering cell death upon interaction with its ligand, galectin-9. TIM-3 contributes to the suppressive tumor microenvironment and its overexpression is associated with poor prognosis in a variety of cancers (see, e.g., Li et al., Acta Oncol. 54:1706-13, 2015). General examples of TIM-3 antagonists or inhibitors include an antibody or antigen-binding fragment or small molecule that specifically binds to TIM-3 and reduces or inhibits one or more of its immune-suppressive activities.

In some embodiments, the agent is a LAG-3 antagonist or inhibitor. Lymphocyte Activation Gene-3 (LAG-3) is expressed on activated T-cells, natural killer cells, B-cells and plasmacytoid dendritic cells. It negatively regulates cellular proliferation, activation, and homeostasis of T-cells, in a similar fashion to CTLA-4 and PD-1 (see, e.g., Workman and Vignali. European Journal of Immun. 33: 970-9, 2003; and Workman et al., Journal of Immun. 172: 5450-5, 2004), and has been reported to play a role in Treg suppressive function (see, e.g., Huang et al., Immunity. 21: 503-13, 2004). LAG3 also maintains CD8+ T-cells in a tolerogenic state and combines with PD-1 to maintain CD8 T-cell exhaustion. General examples of LAG-3 antagonists or inhibitors include an antibody or antigen-binding fragment or small molecule that specifically binds to LAG-3 and inhibits one or more of its immune-suppressive activities. Specific examples include the antibody BMS-986016, and antigen-binding fragments thereof.

In some embodiments, the agent is a BTLA antagonist or inhibitor. B- and T-lymphocyte attenuator (BTLA; CD272) expression is induced during activation of T-cells, and it inhibits T-cells via interaction with tumor necrosis family receptors (TNF-R) and B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses, for example, by inhibiting the function of human CD8+ cancer-specific T-cells (see, e.g., Derré et al., J Clin Invest 120:157-67, 2009). General examples of BTLA antagonists or inhibitors include an antibody or antigen-binding fragment or small molecule that specifically binds to BTLA-4 and reduce one or more of its immune-suppressive activities.

In some embodiments, the agent is an HVEM antagonist or inhibitor, for example, an antagonist or inhibitor that specifically binds to HVEM and interferes with its interaction with BTLA or CD160. General examples of HVEM antagonists or inhibitors include an antibody or antigen-binding fragment or small molecule that specifically binds to HVEM, optionally reduces the HVEM/BTLA and/or HVEM/CD160 interaction, and thereby reduces one or more of the immune-suppressive activities of HVEM.

In some embodiments, the agent is a CD160 antagonist or inhibitor, for example, an antagonist or inhibitor that specifically binds to CD160 and interferes with its interaction with HVEM. General examples of CD160 antagonists or inhibitors include an antibody or antigen-binding fragment or small molecule that specifically binds to CD160, optionally reduces the CD160/HVEM interaction, and thereby reduces or inhibits one or more of its immune-suppressive activities.

In some embodiments, the agent is a TIGIT antagonist or inhibitor. T cell Ig and ITIM domain (TIGIT) is a co-inhibitory receptor that is found on the surface of a variety of lymphoid cells, and suppresses antitumor immunity, for example, via Tregs (Kurtulus et al., J Clin Invest. 125:4053-4062, 2015). General examples of TIGIT antagonists or inhibitors include an antibody or antigen-binding fragment or small molecule that specifically binds to TIGIT and reduce one or more of its immune-suppressive activities (see, e.g., Johnston et al., Cancer Cell. 26:923-37, 2014).

In certain embodiments, the immune checkpoint modulatory agent is an agonist of one or more stimulatory immune checkpoint molecules. Exemplary stimulatory immune checkpoint molecules include CD40, OX40, Glucocorticoid-Induced TNFR Family Related Gene (GITR), CD137 (4-1BB), CD27, CD28, CD226, and Herpes Virus Entry Mediator (HVEM).

In some embodiments, the agent is a CD40 agonist. CD40 is expressed on antigen-presenting cells (APC) and some malignancies. Its ligand is CD40L (CD154). On APC, ligation results in upregulation of costimulatory molecules, potentially bypassing the need for T-cell assistance in an antitumor immune response. CD40 agonist therapy plays an important role in APC maturation and their migration from the tumor to the lymph nodes, resulting in elevated antigen presentation and T cell activation. Anti-CD40 agonist antibodies produce substantial responses and durable anticancer immunity in animal models, an effect mediated at least in part by cytotoxic T-cells (see, e.g., Johnson et al. Clin Cancer Res. 21: 1321-1328, 2015; and Vonderheide and Glennie, Clin Cancer Res. 19:1035-43, 2013). General examples of CD40 agonists include an antibody or antigen-binding fragment or small molecule or ligand that specifically binds to CD40 and increases one or more of its immunostimulatory activities. Specific examples include CP-870,893, dacetuzumab, Chi Lob 7/4, ADC-1013, CD40L, rhCD40L, and antigen-binding fragments thereof. Specific examples of CD40 agonists include, but are not limited to, APX005 (see, e.g., US 2012/0301488) and APX005M (see, e.g., US 2014/0120103).

In some embodiments, the agent is an OX40 agonist. OX40 (CD134) promotes the expansion of effector and memory T cells, and suppresses the differentiation and activity of T-regulatory cells (see, e.g., Croft et al., Immunol Rev. 229:173-91, 2009). Its ligand is OX40L (CD252). Since OX40 signaling influences both T-cell activation and survival, it plays a key role in the initiation of an anti-tumor immune response in the lymph node and in the maintenance of the anti-tumor immune response in the tumor microenvironment. General examples of OX40 agonists include an antibody or antigen-binding fragment or small molecule or ligand that specifically binds to OX40 and increases one or more of its immunostimulatory activities. Specific examples include OX86, OX-40L, Fc-OX40L, GSK3174998, MED10562 (a humanized OX40 agonist), MED16469 (murine OX4 agonist), and MED16383 (an OX40 agonist), and antigen-binding fragments thereof.

In some embodiments, the agent is a GITR agonist. Glucocorticoid-Induced TNFR family Related gene (GITR) increases T cell expansion, inhibits the suppressive activity of Tregs, and extends the survival of T-effector cells. GITR agonists have been shown to promote an anti-tumor response through loss of Treg lineage stability (see, e.g., Schaer et al., Cancer Immunol Res. 1:320-31, 2013). These diverse mechanisms show that GITR plays an important role in initiating the immune response in the lymph nodes and in maintaining the immune response in the tumor tissue. Its ligand is GITRL. General examples of GITR agonists include an antibody or antigen-binding fragment or small molecule or ligand that specifically binds to GITR and increases one or more of its immunostimulatory activities. Specific examples include GITRL, INCAGN01876, DTA-1, MED11873, and antigen-binding fragments thereof.

In some embodiments, the agent is a CD137 agonist. CD137 (4-1BB) is a member of the tumor necrosis factor (TNF) receptor family, and crosslinking of CD137 enhances T-cell proliferation, IL-2 secretion, survival, and cytolytic activity. CD137-mediated signaling also protects T-cells such as CD8+ T-cells from activation-induced cell death. General examples of CD137 agonists include an antibody or antigen-binding fragment or small molecule or ligand that specifically binds to CD137 and increases one or more of its immunostimulatory activities. Specific examples include the CD137 (or 4-1 BB) ligand (see, e.g., Shao and Schwarz, J Leukoc Biol. 89:21-9, 2011) and the antibody utomilumab, including antigen-binding fragments thereof.

In some embodiments, the agent is a CD27 agonist. Stimulation of CD27 increases antigen-specific expansion of naïve T cells and contributes to T-cell memory and long-term maintenance of T-cell immunity. Its ligand is CD70. The targeting of human CD27 with an agonist antibody stimulates T-cell activation and antitumor immunity (see, e.g., Thomas et al., Oncoimmunology. 2014; 3:e27255. doi:10.4161/onci.27255; and He et al J Immunol. 191:4174-83, 2013). General examples of CD27 agonists include an antibody or antigen-binding fragment or small molecule or ligand that specifically binds to CD27 and increases one or more of its immunostimulatory activities. Specific examples include CD70 and the antibodies varlilumab and CDX-1127 (1F5), including antigen-binding fragments thereof.

In some embodiments, the agent is a CD28 agonist. CD28 is constitutively expressed CD4+ T cells some CD8+ T cells. Its ligands include CD80 and CD86, and its stimulation increases T-cell expansion. General examples of CD28 agonists include an antibody or antigen-binding fragment or small molecule or ligand that specifically binds to CD28 and increases one or more of its immunostimulatory activities. Specific examples include CD80, CD86, the antibody TAB08, and antigen-binding fragments thereof.

In some embodiments, the agent is CD226 agonist. CD226 is a stimulating receptor that shares ligands with TIGIT, and opposite to TIGIT, engagement of CD226 enhances T-cell activation (see, e.g., Kurtulus et al., J Clin Invest. 125:4053-4062, 2015; Bottino et al., J Exp Med. 1984:557-567, 2003; and Tahara-Hanaoka et al., Int Immunol. 16:533-538, 2004). General examples of CD226 agonists include an antibody or antigen-binding fragment or small molecule or ligand (e.g., CD112, CD155) that specifically binds to CD226 and increases one or more of its immunostimulatory activities.

In some embodiments, the agent is an HVEM agonist. Herpesvirus entry mediator (HVEM), also known as tumor necrosis factor receptor superfamily member 14 (TNFRSF14), is a human cell surface receptor of the TNF-receptor superfamily. HVEM is found on a variety of cells including T-cells, APCs, and other immune cells. Unlike other receptors, HVEM is expressed at high levels on resting T-cells and down-regulated upon activation. It has been shown that HVEM signaling plays a crucial role in the early phases of T-cell activation and during the expansion of tumor-specific lymphocyte populations in the lymph nodes. General examples of HVEM agonists include an antibody or antigen-binding fragment or small molecule or ligand that specifically binds to HVEM and increases one or more of its immunostimulatory activities.

In certain embodiments, the anti-VISTA antibodies disclosed herein are administered in combination with one or more bi-specific or multi-specific antibodies. For instance, certain bi-specific or multi-specific antibodies are able to (i) bind to and inhibit one or more inhibitory immune checkpoint molecules, and also (ii) bind to and agonize one or more stimulatory immune checkpoint molecules. In certain embodiments, a bi-specific or multi-specific antibody (i) binds to and inhibits one or more of PD-L1, PD-L2, PD-1, CTLA-4, IDO, TDO, TIM-3, LAG-3, BTLA, CD160, and/or TIGIT, and also (ii) binds to and agonizes one or more of CD40, OX40 Glucocorticoid-Induced TNFR Family Related Gene (GITR), CD137 (4-1 BB), CD27, CD28, CD226, and/or Herpes Virus Entry Mediator (HVEM).

In some embodiments, the anti-VISTA antibodies disclosed herein are administered in combination with one or more cancer vaccines. In certain embodiments, the cancer vaccine is selected from one or more of Oncophage, a human papillomavirus HPV vaccine optionally Gardasil or Cervarix, a hepatitis B vaccine optionally Engerix-B, Recombivax HB, or Twinrix, and sipuleucel-T (Provenge), or comprises a cancer antigen selected from one or more of human Her2/neu, Her1/EGF receptor (EGFR), Her3, A33 antigen, B7H3, CD5, CD19, CD20, CD22, CD23 (IgE Receptor), MAGE-3, C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin.

In some embodiments, the anti-VISTA antibodies disclosed herein are administered in combination with one or more oncolytic viruses. In some embodiments, the oncolytic virus selected from one or more of talimogene laherparepvec (T-VEC), coxsackievirus A21 (CAVATAK™), Oncorine (H101), pelareorep (REOLYSIN®), Seneca Valley virus (NTX-010), Seneca virus SVV-001, ColoAd1, SEPREHVIR (HSV-1716), CGTG-102 (Ad5/3-D24-GMCSF), GL-ONC1, MV-NIS, and DNX-2401.

In certain embodiments, the cancer immunotherapy agent is a cytokine. Exemplary cytokines include interferon (IFN)-α, IL-2, IL-12, IL-7, IL-21, and Granulocyte-macrophage colony-stimulating factor (GM-CSF).

In certain embodiments, the cancer immunotherapy agent is cell-based immunotherapy, for example, a T-cell based adoptive immunotherapy. In some embodiments, the cell-based immunotherapy comprises cancer antigen-specific T-cells, optionally ex vivo-derived T-cells. In some embodiments, the cancer antigen-specific T-cells are selected from one or more of chimeric antigen receptor (CAR)-modified T-cells, and T-cell Receptor (TCR)-modified T-cells, tumor infiltrating lymphocytes (TILs), and peptide-induced T-cells. In specific embodiments, the CAR-modified T-cell is targeted against CD-19 (see, e.g., Maude et al., Blood. 125:4017-4023, 2015).

In some embodiments, the anti-VISTA antibodies disclosed herein are used as part of adoptive immunotherapies, for example, autologous immunotherapies. Certain embodiments thus include methods of treating a cancer in a patient in need thereof, comprising:

(a) incubating ex vivo-derived immune cells with an anti-VISTA antibody, or antigen-binding fragment thereof, described herein; and

(b) administering the autologous immune cells to the patient.

In some instances, the ex vivo-derived immune cells are autologous cells, which are obtained from the patient to be treated. In some embodiments, the autologous immune cells comprise lymphocytes, natural killer (NK) cells, macrophages, and/or dendritic cells (DCs). In some embodiments, the lymphocytes comprise T-cells, optionally cytotoxic T-lymphocytes (CTLs). See, for example, June, J Clin Invest. 117: 1466-1476, 2007; Rosenberg and Restifo, Science. 348:62-68, 2015; Cooley et al., Biol. of Blood and Marrow Transplant. 13:33-42, 2007; and Li and Sun, Chin J Cancer Res. 30:173-196, 2018, for descriptions of adoptive T-cell and NK cell immunotherapies. In some embodiments, the T-cells comprise cancer antigen-specific T-cells, which are directed against at least one “cancer antigen”, as described herein. In certain embodiments, the anti-VISTA antibody, or antigen-binding fragment thereof, enhances the efficacy of the adoptively transferred immune cells.

In certain embodiments, the anti-VISTA antibodies disclosed herein may be administered in conjunction with any number of chemotherapeutic agents. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhne-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

A variety of other therapeutic agents may be used in conjunction with the anti-VISTA antibodies described herein. In one embodiment, the antibody is administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

Exemplary NSAIDs are chosen from the group consisting of ibuprofen, naproxen, naproxen sodium, Cox-2 inhibitors such as VIOXX® (rofecoxib) and CELEBREX® (celecoxib), and sialylates. Exemplary analgesics are chosen from the group consisting of acetaminophen, oxycodone, tramadol of proporxyphene hydrochloride. Exemplary glucocorticoids are chosen from the group consisting of cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists (e.g., etanercept (ENBREL®), adalimumab (HUMIRA®) and infliximab (REMICADE®)), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

In certain embodiments, the antibodies described herein are administered in conjunction with a cytokine. By “cytokine” as used herein is meant a generic term for proteins released by one cell population that act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

The compositions comprising herein described VISTA-specific antibodies may be administered to an individual afflicted with a disease as described herein, including, but not limited to cancer, autoimmune diseases, inflammatory diseases, and infectious diseases. Cancers include, but are not limited to, non-Hodgkin's lymphomas, Hodgkin's lymphoma, chronic lymphocytic leukemias, hairy cell leukemias, acute lymphoblastic leukemias, multiple myeloma, carcinomas of the pancreas, colon, gastric intestine, prostate, bladder, kidney ovary, cervix, breast, lung, nasopharynx, and malignant melanoma.

Autoimmune diseases include, but are not limited to, arthritis (including rheumatoid arthritis, reactive arthritis), systemic lupus erythematosus (SLE), psoriasis and inflammatory bowel disease (IBD), encephalomyelitis, uveitis, myasthenia gravis, multiple sclerosis, insulin dependent diabetes, Addison's disease, celiac disease, chronic fatigue syndrome, autoimmune hepatitis, autoimmune alopecia, ankylosing spondylitis, ulcerative colitis, Crohn's disease, fibromyalgia, pemphigus vulgaris, Sjogren's syndrome, Kawasaki's Disease, hyperthyroidism/Graves' disease, hypothyroidism/Hashimoto's disease, endometriosis, scleroderma, pernicious anemia, Goodpasture syndrome, Guillain-Barré syndrome, Wegener's disease, glomerulonephritis, aplastic anemia (including multiply transfused aplastic anemia patients), paroxysmal nocturnal hemoglobinuria, myelodysplastic syndrome, idiopathic thrombocytopenic purpura, autoimmune hemolytic anemia, Evan's syndrome, Factor VIII inhibitor syndrome, systemic vasculitis, dermatomyositis, polymyositis and rheumatic fever, autoimmune lymphoproliferative syndrome (ALPS), autoimmune bullous pemphigoid, Parkinson's disease, sarcoidosis, vitiligo, primary biliary cirrhosis, and autoimmune myocarditis.

Inflammatory diseases include, but are not limited to, Crohn's disease, colitis, dermatitis, psoriasis, diverticulitis, hepatitis, irritable bowel syndrome (IBS), lupus erythematous, nephritis, Parkinson's disease, ulcerative colitis, multiple sclerosis (MS), Alzheimer's disease, arthritis, rheumatoid arthritis, asthma, and various cardiovascular diseases such as atherosclerosis and vasculitis. In certain embodiments, the inflammatory disease is selected from the group consisting of rheumatoid arthritis, diabetes, gout, cryopyrin-associated periodic syndrome, and chronic obstructive pulmonary disorder.

In this regard, one embodiment provides a method of treating, reducing the severity of or preventing inflammation or an inflammatory disease by administering to a patient in need thereof a therapeutically effective amount of a herein disclosed composition comprising agonistic anti-VISTA antibodies. One embodiment provides a method of treating, reducing the severity of or preventing graft-versus-host disease by administering to a transplant patient in need thereof a therapeutically effective amount of a herein disclosed composition comprising agonistic anti-VISTA antibodies. One embodiment provides a method of treating, reducing the severity of or preventing graft rejection by administering to a transplant patient in need thereof a therapeutically effective amount of a herein disclosed composition comprising agonistic anti-VISTA antibodies.

Certain embodiments provide a method of treating, reducing the severity of or preventing an infectious disease, by administering to a patient in need thereof a therapeutically effective amount of a herein disclosed composition comprising agonistic anti-VISTA antibodies. Infectious diseases include, but are not limited to, viral, bacterial, fungal optionally yeast, and protozoal infections.

For in vivo use for the treatment of human disease, the antibodies described herein are generally incorporated into a pharmaceutical composition prior to administration. A pharmaceutical composition comprises one or more of the antibodies described herein in combination with a physiologically acceptable carrier or excipient as described elsewhere herein. To prepare a pharmaceutical composition, an effective amount of one or more of the compounds is mixed with any pharmaceutical carrier(s) or excipient known to those skilled in the art to be suitable for the particular mode of administration. A pharmaceutical carrier may be liquid, semi-liquid or solid. Solutions or suspensions used for parenteral, intradermal, subcutaneous or topical application may include, for example, a sterile diluent (such as water), saline solution, fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously, suitable carriers include physiological saline or phosphate buffered saline (PBS), and solutions containing thickening and solubilizing agents, such as glucose, polyethylene glycol, polypropylene glycol and mixtures thereof.

The compositions comprising VISTA-specific antibodies as described herein may be prepared with carriers that protect the antibody against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

Provided herein are methods of treatment using the antibodies that bind VISTA. In one embodiment, an antibody of the present disclosure is administered to a patient having a disease involving inappropriate expression of VISTA, which is meant in the context of the present disclosure to include diseases and disorders characterized by aberrant VISTA expression or activity, due for example to alterations (e.g., statistically significant increases or decreases) in the amount of a protein present, or the presence of a mutant protein, or both. An overabundance may be due to any cause, including but not limited to overexpression at the molecular level, prolonged or accumulated appearance at the site of action, or increased (e.g., in a statistically significant manner) activity of VISTA relative to that which is normally detectable. Such an overabundance of VISTA can be measured relative to normal expression, appearance, or activity of VISTA signaling events, and said measurement may play an important role in the development and/or clinical testing of the antibodies described herein.

In particular, the present antibodies are useful for the treatment of a variety of cancers associated with the expression of VISTA. For example, one embodiment provides a method for the treatment of a cancer including, but not limited to, non-Hodgkin's lymphomas, Hodgkin's lymphoma, chronic lymphocytic leukemias, hairy cell leukemias, acute lymphoblastic leukemias, multiple myeloma, carcinomas of the pancreas, colon, gastric intestine, prostate, bladder, kidney, ovary, cervix, breast, lung, nasopharynx, and malignant melanoma, by administering to a cancer patient a therapeutically effective amount of a herein disclosed VISTA-specific antibody. An amount that, following administration, inhibits, prevents or delays the progression and/or metastasis of a cancer in a statistically significant manner (i.e., relative to an appropriate control as will be known to those skilled in the art) is considered effective.

Another embodiment provides a method for preventing metastasis of a cancer including, but not limited to, non-Hodgkin's lymphomas, Hodgkin's lymphoma, chronic lymphocytic leukemias, hairy cell leukemias, acute lymphoblastic leukemias, multiple myeloma, carcinomas of the pancreas, colon, gastric intestine, prostate, bladder, kidney, ovary, cervix, breast, lung, nasopharynx, and malignant melanoma, by administering to a cancer patient a therapeutically effective amount of a herein disclosed VISTA-specific antibody (e.g., an amount that, following administration, inhibits, prevents or delays metastasis of a cancer in a statistically significant manner, i.e., relative to an appropriate control as will be known to those skilled in the art).

Another embodiment provides a method for preventing a cancer including, but not limited to, non-Hodgkin's lymphomas, Hodgkin's lymphoma, chronic lymphocytic leukemias, hairy cell leukemias, acute lymphoblastic leukemias, multiple myeloma, carcinomas of the pancreas, colon, gastric intestine, prostate, bladder, kidney, ovary, cervix, breast, lung, nasopharynx, and malignant melanoma, by administering to a cancer patient a therapeutically effective amount of a herein disclosed VISTA-specific antibody.

Another embodiment provides a method for treating, inhibiting the progression of or prevention of non-Hodgkin's lymphomas, Hodgkin's lymphoma, chronic lymphocytic leukemias, hairy cell leukemias, acute lymphoblastic leukemias, multiple myeloma, carcinomas of the pancreas, colon, gastric intestine, prostate, bladder, kidney, ovary, cervix, breast, lung, nasopharynx, or malignant melanoma by administering to a patient afflicted by one or more of these diseases a therapeutically effective amount of a herein disclosed VISTA-specific antibody.

In another embodiment, anti-VISTA antibodies are used to determine the structure of bound antigen, e.g., conformational epitopes, which structure may then be used to develop compounds having or mimicking this structure, e.g., through chemical modeling and SAR methods.

Various other embodiments relate, in part, to diagnostic applications for detecting the presence of cells or tissues expressing VISTA. Thus, the present disclosure provides methods of detecting VISTA in a sample, such as detection of cells or tissues expressing VISTA. Such methods can be applied in a variety of known detection formats, including, but not limited to immunohistochemistry (IHC), immunocytochemistry (ICC), in situ hybridization (ISH), whole-mount in situ hybridization (WISH), fluorescent DNA in situ hybridization (FISH), flow cytometry, enzyme immuno-assay (EIA), and enzyme linked immuno-assay (ELISA).

ISH is a type of hybridization that uses a labeled complementary DNA or RNA strand (i.e., primary binding agent) to localize a specific DNA or RNA sequence in a portion or section of a cell or tissue (in situ), or if the tissue is small enough, the entire tissue (whole mount ISH). One having ordinary skill in the art would appreciate that this is distinct from immunohistochemistry, which localizes proteins in tissue sections using an antibody as a primary binding agent. DNA ISH can be used on genomic DNA to determine the structure of chromosomes. Fluorescent DNA ISH (FISH) can, for example, be used in medical diagnostics to assess chromosomal integrity. RNA ISH (hybridization histochemistry) is used to measure and localize mRNAs and other transcripts within tissue sections or whole mounts.

In various embodiments, the antibodies described herein are conjugated to a detectable label that may be detected directly or indirectly. In this regard, an antibody “conjugate” refers to an anti-VISTA antibody that is covalently linked to a detectable label. In the present disclosure, DNA probes, RNA probes, monoclonal antibodies, antigen-binding fragments thereof, and antibody derivatives thereof, such as a single-chain-variable-fragment antibody or an epitope tagged antibody, may all be covalently linked to a detectable label. In “direct detection”, only one detectable antibody is used, i.e., a primary detectable antibody. Thus, direct detection means that the antibody that is conjugated to a detectable label may be detected, per se, without the need for the addition of a second antibody (secondary antibody).

A “detectable label” is a molecule or material that can produce a detectable (such as visually, electronically or otherwise) signal that indicates the presence and/or concentration of the label in a sample. When conjugated to an antibody, the detectable label can be used to locate and/or quantify the target to which the specific antibody is directed. Thereby, the presence and/or concentration of the target in a sample can be detected by detecting the signal produced by the detectable label. A detectable label can be detected directly or indirectly, and several different detectable labels conjugated to different specific-antibodies can be used in combination to detect one or more targets.

Examples of detectable labels, which may be detected directly, include fluorescent dyes and radioactive substances and metal particles. In contrast, indirect detection requires the application of one or more additional antibodies, i.e., secondary antibodies, after application of the primary antibody. Thus, the detection is performed by the detection of the binding of the secondary antibody or binding agent to the primary detectable antibody. Examples of primary detectable binding agents or antibodies requiring addition of a secondary binding agent or antibody include enzymatic detectable binding agents and hapten detectable binding agents or antibodies.

In some embodiments, the detectable label is conjugated to a nucleic acid polymer which comprises the first binding agent (e.g., in an ISH, WISH, or FISH process). In other embodiments, the detectable label is conjugated to an antibody which comprises the first binding agent (e.g., in an IHC process).

Examples of detectable labels which may be conjugated to antibodies used in the methods of the present disclosure include fluorescent labels, enzyme labels, radioisotopes, chemiluminescent labels, electrochemiluminescent labels, bioluminescent labels, polymers, polymer particles, metal particles, haptens, and dyes.

Examples of fluorescent labels include 5-(and 6)-carboxyfluorescein, 5- or 6-carboxyfluorescein, 6-(fluorescein)-5-(and 6)-carboxamido hexanoic acid, fluorescein isothiocyanate, rhodamine, tetramethylrhodamine, and dyes such as Cy2, Cy3, and Cy5, optionally substituted coumarin including AMCA, PerCP, phycobiliproteins including R-phycoerythrin (RPE) and allophycoerythrin (APC), Texas Red, Princeton Red, green fluorescent protein (GFP) and analogues thereof, and conjugates of R-phycoerythrin or allophycoerythrin, inorganic fluorescent labels such as particles based on semiconductor material like coated CdSe nanocrystallites.

Examples of polymer particle labels include micro particles or latex particles of polystyrene, PMMA or silica, which can be embedded with fluorescent dyes, or polymer micelles or capsules which contain dyes, enzymes or substrates.

Examples of metal particle labels include gold particles and coated gold particles, which can be converted by silver stains. Examples of haptens include DNP, fluorescein isothiocyanate (FITC), biotin, and digoxigenin. Examples of enzymatic labels include horseradish peroxidase (HRP), alkaline phosphatase (ALP or AP), β-galactosidase (GAL), glucose-6-phosphate dehydrogenase, β-N-acetylglucosamimidase, β-glucuronidase, invertase, Xanthine Oxidase, firefly luciferase and glucose oxidase (GO). Examples of commonly used substrates for horseradishperoxidase include 3,3′-diaminobenzidine (DAB), diaminobenzidine with nickel enhancement, 3-amino-9-ethylcarbazole (AEC), Benzidine dihydrochloride (BDHC), Hanker-Yates reagent (HYR), Indophane blue (IB), tetramethylbenzidine (TMB), 4-chloro-1-naphtol (CN), alpha-naphtol pyronin (.alpha.-NP), o-dianisidine (OD), 5-bromo-4-chloro-3-indolylphosp-hate (BCIP), Nitro blue tetrazolium (NBT), 2-(p-iodophenyl)-3-p-nitropheny-I-5-phenyl tetrazolium chloride (INT), tetranitro blue tetrazolium (TNBT), 5-bromo-4-chloro-3-indoxyl-beta-D-galactoside/ferro-ferricyanide (BCIG/FF).

Examples of commonly used substrates for Alkaline Phosphatase include Naphthol-AS-B 1-phosphate/fast red TR (NABP/FR), Naphthol-AS-MX-phosphate/fast red TR (NAMP/FR), Naphthol-AS-B1-phosphate/-fast red TR (NABP/FR), Naphthol-AS-MX-phosphate/fast red TR (NAMP/FR), Naphthol-AS-B1-phosphate/new fuschin (NABP/NF), bromochloroindolyl phosphate/nitroblue tetrazolium (BCIP/NBT), 5-Bromo-4-chloro-3-indolyl-b-d-galactopyranoside (BCIG).

Examples of luminescent labels include luminol, isoluminol, acridinium esters, 1,2-dioxetanes and pyridopyridazines. Examples of electrochemiluminescent labels include ruthenium derivatives. Examples of radioactive labels include radioactive isotopes of iodide, cobalt, selenium, tritium, carbon, sulfur and phosphorous.

Detectable labels may be linked to the antibodies described herein or to any other molecule that specifically binds to a biological marker of interest, e.g., an antibody, a nucleic acid probe, or a polymer. Furthermore, one of ordinary skill in the art would appreciate that detectable labels can also be conjugated to second, and/or third, and/or fourth, and/or fifth binding agents or antibodies, etc. Moreover, the skilled artisan would appreciate that each additional binding agent or antibody used to characterize a biological marker of interest may serve as a signal amplification step. The biological marker may be detected visually using, e.g., light microscopy, fluorescent microscopy, electron microscopy where the detectable substance is for example a dye, a colloidal gold particle, a luminescent reagent. Visually detectable substances bound to a biological marker may also be detected using a spectrophotometer. Where the detectable substance is a radioactive isotope detection can be visually by autoradiography, or non-visually using a scintillation counter. See, e.g., Larsson, 1988, Immunocytochemistry: Theory and Practice, (CRC Press, Boca Raton, Fla.); Methods in Molecular Biology, vol. 80 1998, John D. Pound (ed.) (Humana Press, Totowa, N.J.).

The disclosure further provides kits for detecting VISTA or cells or tissues expressing VISTA in a sample, wherein the kits contain at least one antibody, polypeptide, polynucleotide, vector or host cell as described herein. In certain embodiments, a kit may comprise buffers, enzymes, labels, substrates, beads or other surfaces to which the antibodies of the disclosure are attached, and the like, and instructions for use.

### EXAMPLES

### Example 1

### Production and Humanization of Anti-Vista Antibodies

New Zealand white rabbits were immunized with recombinant human VISTA and VISTA-expressing cells. All rabbits had high serum titers of specific binding to human VISTA and were sacrificed for cell fusion. Twenty-six hybridomas that were positive for binding to VISTA as measured by an ELISA binding assay were selected for functional screening. The amino acid sequences of the VH and VL regions of the 26 clones are aligned in FIG. 1. Some of the antibodies had different VH regions that paired with the same VL region as shown in Table 4 below.

TABLE 4

| | Rabbit VH | | Rabbit VL |
|---|---|---|---|
| Clone | SEQ ID NO: | Clone | SEQ ID NO: |
| 2D12H1 | 1 | 2D12L1 | 2 |
| 3A5H1 | 9 | 3A5L2 | 10 |
| 14D8H2 | 17 | 14D8L1 | 18 |
| 14F1H1 | 25 | 14F1L1 | 26 |
| 29G7H1 | 33 | 29G7L1 | 34 |
| 41A11H1 | 41 | 41A11L2 | 42 |
| 2H2 | 49 | 2L3 | 70 |
| 2H4 | 50 | | |
| 3H1 | 51 | 3L1 | 71 |
| 3H2 | 52 | | |
| 5H2 | 53 | 5L3 | 72 |
| 5H4 | 54 | | |
| 1H1 | 55 | 1L2 | 73 |
| 1H4 | 56 | | |
| 3A9-H1 | 58 | 3A9-L2 | 74 |
| 19B5-H2 | 59 | 19B5-L1 | 75 |
| 10D4H2 | 60 | 10D4-L1 | 76 |
| 32B7-H1 | 61 | 32B7-L1 | 77 |
| 9A3-H1 | 62 | 9A3-L1 | 78 |
| 34C3-H2 | 63 | 34C3-L1 | 79 |
| 5C12-H3 | 64 | 5C12-L1 | 80 |
| 18D3-H4 | 65 | 18D3-L2 | 81 |
| 15H1 | 66 | 15L1 | 82 |
| 4A2-H4 | 67 | 4A2-L3 | 83 |
| 5B6-H4 | 68 | 5B6-L1 | 84 |
| 6B5-H4 | 69 | 6B5-L1 | 85 |

Six of the clones, 2D12, 3A5, 14D8, 14F1, 29G7, and 41A11, were selected for molecular cloning and recombinant expression for functional characterization.

### Functional Screening of Recombinant Anti-VISTA Antibodies

Numerous assays were used to characterize the potency of the selected anti-VISTA antibodies identified above. The selected antibodies were converted from rabbit mAb to chimeric mAb with rabbit Fab and human IgG1. The anti-VISTA human IgG1 antibody, VSTB112, was chosen as the benchmark for comparison studies. VSTB112 is described in WO 2015/097536 and WO 2016/207717.

A competition ELISA assay with VSTB112 was performed. 96 well plates were coated with VISTA-his protein. Human IgG1 chimeric anti-VISTA antibodies were incubated with protein for 1 hour. Biotin-labeled VSTB112 was added to the wells and incubated for an additional hour. Wells were washed and binding of VSTB112 was detected using streptavidin-HRP as shown in FIG. 2. These results showed that S2D12 binds to a distinctly different epitope on VISTA in comparison to VSTB112, and 29G7 also showed a differential binding epitope in comparison to VSTB112.

The ability of chimeric VISTA monoclonal antibodies to upregulate Major Histocompatibility Complex (MHC) Class II molecules on monocytes was tested. Human PBMC were isolated from buffy coats by Ficoll density centrifugation. Monocytes were enriched from human PBMC using CD14 microbeads and plated at 100,000 cells per well in a round-bottom 96-well plate. Anti-VISTA antibodies were added at 0.02, 0.2, 2, and 20 nM concentrations, and plates were incubated for 48 hours at 37° C. Cells were harvested and analyzed for pan-MHCII (DP,DQ, DR) expression by flow cytometry (FIG. 3). As shown in FIG. 3, all 6 of the chimeric VISTA antibodies induced greater expression of MHCII in comparison to VSTB112.

The 6 clones, 2D12, 3A5, 14D8, 14F1, 29G7, and 41A11, were humanized using a proprietary mutational lineage guided (MLG) humanization technology (see e.g., U.S. Pat. No. 7,462,697). The humanization provided two candidates from the 29G7 clone, namely 29G7-HZD2 and 29G7-HZD4. The amino acid sequences of the 7 sets of humanized VH and VL regions are set forth in SEQ ID NOs:86-99. The humanized sequences are summarized in Table 3 above.

### Functional Screening of Humanized Candidate Anti-VISTA Antibodies

Numerous in vitro assays were used to characterize the potency of the 7 humanized candidate anti-VISTA antibodies identified above. First, the humanized anti-VISTA antibodies were tested for binding to soluble VISTA as shown in FIG. 4.

In order to examine the ability of the 7 humanized anti-VISTA antibodies to bind to cell surface expressed VISTA, HEK293 cells were transfected with human VISTA cDNA for 24 hours using Fugene 6 transfection reagent. Cells were harvested by trypsinization, re-suspended in FACS buffer and plated in 96-well plates. Cells were incubated with the concentration of anti-VISTA antibodies indicated in FIG. 5 for 30 minutes on ice. Antibodies were detected using anti-human Ig antibodies and binding was assessed on the MACSQuant flow cytometer (FIG. 5). The binding affinities of the 7 humanized anti-VISTA antibodies are provided in Table 5 below.

TABLE 5
Binding Affinity of Anti-VISTA Antibodies

| Clone | KD (nM) |
|---|---|
| 29G7-HZD2 | 0.2805 |
| 29G7-HZD4 | 0.2953 |
| 2D12-HZD3 | 1.307 |
| 3A5-HZD | 0.2928 |
| 14D8-HZD2 | 2.2 |
| 14F1-HZD2 | 2.195 |
| 41A11-HZD2 | 1.35 |

The ability of humanized anti-VISTA antibodies to enhance immune responses was tested in Staphylococcus enterotoxin B (SEB) stimulation assays. Human PBMC were isolated from buffy coats using Ficoll density centrifugation. PBMC were plated at 200,000 cells per well of a 96-well flat-bottom plate. VISTA antibodies were added at the concentrations indicated in FIG. 6. Finally, SEB was added at 10 ng/mL and plates were incubated for 4 days at 37° C. After incubation, supernatants were harvested and analyzed for IFN-γ by ELISA (FIG. 6). As shown in FIG. 6, addition of 29G7-HZD4, 29G7-HZD2, 2D12-HZD3, 3A5-HZD, and 41A11-HZD2 resulted in increased production of IFNγ, demonstrating that these humanized anti-VISTA antibodies are VISTA antagonists.

Next, humanized anti-VISTA antibodies were tested for enhancement of a mixed lymphocyte reaction (MLR). Briefly, human monocytes were isolated from PBMC using CD14 microbeads and differentiated into dendritic cells in complete media containing 50 ng/mL of human GM-CSF and 100 ng/mL of human IL-4 for 7 days. CD4 T cells were enriched from human PBMC using CD4 microbeads and labeled with Cell Trace Violet. Monocyte-derived dendritic cells (moDC) were seeded at 50,000 cells per well in a flat-bottom 96-well plate. Antibodies were added at the concentrations indicated in FIG. 7. Finally, enriched and violet-labeled HLA-mismatched CD4 T cells were added at 200,000 per well and co-cultured in 200 μL of media for 5 days at 37° C.

Cells were harvested and analyzed to determine the percentage of proliferation (FIG. 7). As shown in FIG. 7, addition of 29G7-HZD2, 29G7-HZD4, 2D12, 14D8-HZD2 and 41A11-HZD2 increased T cell proliferation in response to alloantigen indicating that these anti-VISTA antibodies can reverse VISTA-mediated immune suppression.

### Example 2

### Biological Activity of Anti-Vista Antibodies

The ability of human chimeric VISTA monoclonal antibodies activate human NK cells was tested. Human PBMC were isolated from buffy coats by Ficoll density centrifugation and plated at 200,000 cells per well in a round-bottom 96-well plate. Anti-VISTA antibodies were added at the indicated concentrations and plates were incubated for 24 hours at 37 C. Supernatants were analyzed for IFN-gamma production by ELISA. Cells were harvested and analyzed for CD69 and CD25 expression on CD56+NK cells by flow cytometry.

As shown in FIGS. 8A-8C, anti-VISTA antibodies induced NK cell activation and IFN-gamma secretion.

The ability of humanized anti-VISTA antibodies to induce cytokine release in whole blood assay was also tested. Human whole blood was plated at 200 uL per well of a 96-well round-bottom plate. VISTA antibodies were added at 100, 10, 1 and 0.1 nM. Plates were incubated for 24 hours at 37° C. After incubation, plates were centrifuged at 2000 RPM for 5 minutes and plasma was collected for cytokine and chemokine analysis using Luminex.

Table 6 below illustrates representative data from 6 donors. (DC=dendritic cell; MM=monocyte/macrophage; L=lymphocyte).

TABLE 6

| Analyte | Upregulated | No Effect | Undetected | Source |
|---|---|---|---|---|
| IL-12p40 | | + | | DC |
| IL-12p70 | | + | | DC |
| IL-15 | | + | | DC/MM |
| IFN-a2 | | + | | L |
| IFN-g | ++ | | | L |
| IL-17a | | + | | L |
| TNFb | | | + | L |
| IL-13 | | | + | L |
| IL-2 | | + | | L |
| IL-3 | | + | | L |
| IL-4 | | + | | L |
| IL-5 | | + | | L |
| GM-CSF | | + | | L/MM |
| IL-10 | | + | | L/MM/DC |
| IL-6 | + | | | L/MM/other |
| G-CSF | | | + | MM |
| IL-1ra | + | | | MM |
| IL-1a | + | | | MM |
| IL-1b | | + | | MM |
| IL-8 | + | | | MM |
| MIP-1a | ++ | | | MM |
| MIP-1b | ++ | | | MM |
| IP-10 | +++ | | | MM/L/other |
| TNFa | + | | | MM/L/other |
| MCP-1 | ++ | | | MM/other |
| EGF | | + | | other |
| Eotaxin | | + | | other |
| IL-7 | | + | | other |
| VEGF | | + | | other |

As shown in FIGS. 9A-9I, anti-VISTA antibodies can induce cytokine secretion from cells in whole blood cultures. Cytokines induced by anti-VISTA antibodies include IFN-gamma, IL-6, IL-1ra, IL-1a, IL-8, MIP-1a, MIP-1b IP-10, TNF-alpha and MCP-1, most of which are secreted by myeloid-derived cell types.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An isolated antibody, or an antigen-binding fragment thereof, that binds to VISTA, comprising
(i) a heavy chain variable region comprising a VHCDR1, a VHCDR2, and a VHCDR3 of SEQ ID NOs: 3-5, 11-13, 19-21, 2-29, 35-37, or 43-45; and
(ii) a light chain variable region comprising a VLCDR1, a VLCDR2, and a VLCDR3 of SEQ ID NOs: 6-8, 14-16, 22-24, 30-32, 38-40, or 46-48, respectively;
or a variant of said antibody, or an antigen-binding fragment thereof, comprising heavy and light chain variable regions identical to the heavy and light chain variable regions of (i) and (ii) except for up to 8 amino acid substitutions in said CDR regions.

2. The isolated antibody, or antigen-binding fragment thereof, of claim 0 wherein the heavy chain variable region comprises the amino acid sequence set forth in SEQ ID NO:1, 9, 17, 25, 33, or 41, respectively.

3. The isolated antibody, or antigen-binding fragment thereof, of claim 0 or 2 wherein the light chain variable region comprises the amino acid sequence set forth in SEQ ID NO:2, 10, 18, 26, 34, or 42, respectively.

4. An isolated antibody, or an antigen-binding fragment thereof, that binds to human VISTA, comprising a heavy chain variable region comprising the amino acid sequence set forth in SEQ ID NO:1, 9, 17, 25, 33, or 41.

5. The isolated antibody, or antigen-binding fragment thereof, of claim 0 comprising a light chain variable region which comprises an amino acid sequence having at least 90% identity to the amino acid sequence set forth in SEQ ID NO: 2, 10, 18, 26, 34, or 42, respectively.

6. The isolated antibody, or an antigen-binding fragment thereof, of claim 0 comprising a light chain variable region which comprises the amino acid sequence set forth in SEQ ID NO: 2, 10, 18, 26, 34, or 42, respectively.

7. An isolated antibody, or an antigen-binding fragment thereof, that binds to human VISTA, comprising a light chain variable region comprising the amino acid sequence set forth in SEQ ID NO: 2, 10, 18, 26, 34, or 42, respectively.

8. The isolated antibody, or antigen binding fragment thereof, of claim 0 comprising a heavy chain variable region which comprises an amino acid sequence having at least 90% identity to the amino acid sequence set forth in SEQ ID NO: 1, 9, 17, 25, 33, or 41, respectively.

9. The isolated antibody, or antigen binding fragment thereof, of any one of claims 0-8, wherein the antibody is humanized.

10. The isolated antibody, or antigen binding fragment thereof, of claim 0, wherein the VH region comprises SEQ ID NO:86, 88, 90, 92, 94, 96, or 98 and the VL region comprises SEQ ID NO:87, 89, 91, 93, 95, 97, or 99, respectively.

11. The isolated antibody, or antigen binding fragment thereof, of any one of claims 0-10, wherein the antibody is selected from the group consisting of a single chain antibody, a scFv, a univalent antibody lacking a hinge region, and a minibody.

12. The isolated antibody, or antigen binding fragment thereof, of any one of claims 0-10, wherein the antibody is a Fab or a Fab′ fragment.

13. The isolated antibody, or antigen binding fragment thereof, of any one of claims 0-10, wherein the antibody is a F(ab′)₂ fragment.

14. The isolated antibody, or antigen binding fragment thereof, of any one of claims 0-10, wherein the antibody is a whole antibody.

15. The isolated antibody, or antigen binding fragment thereof, of any one of claims 0-14, comprising a human IgG constant domain.

16. The isolated antibody, or antigen binding fragment thereof, of claim 0, wherein the IgG constant domain comprises an IgG1 CH1 domain.

17. The isolated antibody, or antigen binding fragment thereof, of claim 0, wherein the IgG constant domain comprises an IgG1 Fc region.

18. The isolated antibody, or antigen binding fragment thereof, of any one of claims 1-17, comprising a modified Fc region, wherein the modified Fc region has altered binding affinity for a specific FcγR, increased serum half-life, and/or altered effector function selected from one or more or of complement-dependent cytotoxicity (CDC), antibody-dependent cell-mediated cytotoxicity (ADCC), and antibody-dependent cell-mediated phagocytosis (ADCP), optionally wherein the antibody is cross-linked to alter its effector function.

19. The isolated antibody, or antigen binding fragment thereof, of claim 18, wherein the wherein the modified Fc region has enhanced binding affinity for a specific FcγR, and/or wherein the modified Fc region has enhanced effector function.

20. The isolated antibody, or antigen binding fragment thereof, of claim 18, wherein the wherein the modified Fc region has decreased binding affinity for a specific FcγR, and/or wherein the modified Fc region has decreased effector function.

21. The isolated antibody, or antigen-binding fragment thereof, of any one of claims 1-20 that binds VISTA with a K_{D} of 2.2 nM or lower.

22. The isolated antibody, or antigen-binding fragment thereof, of any one of claims 1-21, wherein the isolated antibody, or antigen-binding fragment thereof:
(a) increases T cell activation;
(b) increases T cell proliferation;
(c) increases MHC II expression;
(d) activates natural killer (NK) cells;
(e) activates monocytes/macrophages;
increases cytokine production, optionally wherein the cytokine is selected from one or more of IFN-gamma, IL-6, IL-1ra, IL-1a, IL-8, MIP-1a, MIP-1 b IP-10, TNF-alpha and MCP-1; or
(g) a combination of any one or more of (a)-(f).

23. The isolated antibody, or antigen-binding fragment thereof, of any one of claims 1-22, that is a VISTA antagonist.

24. The isolated antibody, or antigen-binding fragment thereof, of any one of claims 1-22, that is a VISTA agonist.

25. An isolated antibody, or an antigen-binding fragment thereof, that binds to VISTA, comprising (i) a heavy chain variable region comprising the VHCDR1, VHCDR2, and VHCDR3 of any one of the VH regions shown in FIG. 1; and (ii) a light chain variable region comprising the VLCDR1, the VLCDR2, and the VLCDR3 region of the corresponding VL region of any one of the antibodies shown in FIG. 1; or a variant of said antibody, or an antigen-binding fragment thereof, comprising heavy and light chain variable regions identical to the heavy and light chain variable regions of (i) and (ii) except for up to 8 amino acid substitutions in said CDR regions.

26. An isolated antibody, or an antigen-binding fragment thereof, that binds to VISTA, comprising a heavy chain variable region comprising any one of the VH regions shown in FIG. 1.

27. The isolated antibody, or antigen-binding fragment thereof, of claim 0, further comprising a light chain variable region comprising an amino acid sequence having at least 90% identity to the corresponding VL region as shown in FIG. 1.

28. The isolated antibody, or antigen-binding fragment thereof, of claim 26, further comprising the corresponding light chain variable region as shown in FIG. 1.

29. An isolated antibody, or an antigen-binding fragment thereof, that binds to VISTA, comprising a light chain variable region comprising any one of the VL regions shown in FIG. 1.

30. The isolated antibody, or antigen-binding fragment thereof, of claim 29, further comprising a heavy chain variable region comprising an amino acid sequence having at least 90% identity to the corresponding VH region as shown in FIG. 1.

31. An isolated polynucleotide encoding the isolated antibody, or antigen-binding fragment thereof, according to any one of claims 1-30.

32. An expression vector comprising the isolated polynucleotide of claim 31.

33. An isolated host cell comprising the vector of claim 32.

34. A composition comprising a physiologically acceptable carrier and a therapeutically effective amount of the isolated antibody or antigen-binding fragment thereof according to any one of claims 1-30.

35. A method for treating a patient having a cancer associated with aberrant VISTA expression, comprising administering to the patient a composition of claim 34, thereby treating the cancer associated with aberrant VISTA expression.

36. A method for treating a patient having a cancer associated with VISTA-mediated immune suppression, comprising administering to the patient a composition of claim 34, thereby treating the cancer associated with VISTA-mediated immune suppression.

37. The method of claim 35 or 36, wherein the cancer is selected from one or more of non-Hodgkin's lymphomas, Hodgkin's lymphoma, chronic lymphocytic leukemias, hairy cell leukemias, acute lymphoblastic leukemias, multiple myeloma, carcinomas of the pancreas, colon, gastric intestine, prostate, bladder, kidney ovary, cervix, breast, lung, nasopharynx, and malignant melanoma.

38. The method of any one of claims 35-37, comprising administering to the patient at least one cancer immunotherapy agent.

39. The method of claim 38, wherein the at least one cancer immunotherapy agent is selected from one or more of an immune checkpoint modulatory agent, a cancer vaccine, an oncolytic virus, a cytokine, and a cell-based immunotherapies.

40. The method of claim 39, wherein the immune checkpoint modulatory agent is a polypeptide, optionally an antibody or antigen-binding fragment thereof or a ligand, or a small molecule.

41. The method of claim 39 or 40, wherein the immune checkpoint modulatory agent comprises
(a) an antagonist of a inhibitory immune checkpoint molecule; or
(b) an agonist of a stimulatory immune checkpoint molecule,
optionally, wherein the immune checkpoint modulatory agent specifically binds to the immune checkpoint molecule.

42. The method of claim 41, wherein the inhibitory immune checkpoint molecule is selected from one or more of Programmed Death-Ligand 1 (PD-L1), Programmed Death 1 (PD-1), Programmed Death-Ligand 2 (PD-L2), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), B and T Lymphocyte Attenuator (BTLA), CD160, Herpes Virus Entry Mediator (HVEM), and T-cell immunoreceptor with Ig and ITIM domains (TIGIT).

43. The method of claim 42, wherein:
the antagonist is a PD-L1 and/or PD-L2 antagonist optionally selected from one or more of an antibody or antigen-binding fragment or small molecule that specifically binds thereto, atezolizumab (MPDL3280A), avelumab (MSB0010718C), and durvalumab (MED14736), optionally wherein the cancer is selected from one or more of colorectal cancer, melanoma, breast cancer, non-small-cell lung carcinoma, bladder cancer, and renal cell carcinoma;
the antagonist is a PD-1 antagonist optionally selected from one or more of an antibody or antigen-binding fragment or small molecule that specifically binds thereto, nivolumab, pembrolizumab, MK-3475, AMP-224, AMP-514PDR001, and pidilizumab, optionally wherein the PD-1 antagonist is nivolumab and the cancer is optionally selected from one or more of Hodgkin's lymphoma, melanoma, non-small cell lung cancer, hepatocellular carcinoma, renal cell carcinoma, and ovarian cancer;
the PD-1 antagonist is pembrolizumab and the cancer is optionally selected from one or more of melanoma, non-small cell lung cancer, small cell lung cancer, head and neck cancer, and urothelial cancer;
the antagonist is a CTLA-4 antagonist optionally selected from one or more of an antibody or antigen-binding fragment or small molecule that specifically binds thereto, ipilimumab, tremelimumab, optionally wherein the cancer is selected from one or more of melanoma, prostate cancer, lung cancer, and bladder cancer;
the antagonist is an IDO antagonist optionally selected from one or more of an antibody or antigen-binding fragment or small molecule that specifically binds thereto, indoximod (NLG-8189), 1-methyl-tryptophan (1MT), β-Carboline (norharmane; 9H-pyrido[3,4-b]indole), rosmarinic acid, and epacadostat, and wherein the cancer is optionally selected from one or more of metastatic breast cancer and brain cancer optionally glioblastoma multiforme, glioma, gliosarcoma or malignant brain tumor;
the antagonist is a TDO antagonist optionally selected from one or more of an antibody or antigen-binding fragment or small molecule that specifically binds thereto, 680C91, and LM10;
the antagonist is a TIM-3 antagonist optionally selected from one or more of an antibody or antigen-binding fragment or small molecule that specifically binds thereto;
the antagonist is a LAG-3 antagonist optionally selected from one or more of an antibody or antigen-binding fragment or small molecule that specifically binds thereto, and BMS-986016;
the antagonist is a BTLA, CD160, and/or HVEM antagonist optionally selected from one or more of an antibody or antigen-binding fragment or small molecule that specifically binds thereto; and/or
the antagonist is a TIGIT antagonist optionally selected from one or more of an antibody or antigen-binding fragment or small molecule that specifically binds thereto.

44. The method of claim 41, wherein the stimulatory immune checkpoint molecule is selected from one or more of CD40, OX40, Glucocorticoid-Induced TNFR Family Related Gene (GITR), CD137 (4-1BB), CD27, CD28, CD226, and Herpes Virus Entry Mediator (HVEM).

45. The method of claim 44, wherein:
the agonist is a CD40 agonist optionally selected from one or more of an antibody or antigen-binding fragment or small molecule or ligand that specifically binds thereto, APX005, APX005M, CP-870,893, dacetuzumab, Chi Lob 7/4, ADC-1013, and rhCD40L, and wherein the cancer is optionally selected from one or more of melanoma, pancreatic carcinoma, mesothelioma, and hematological cancers optionally lymphoma such as Non-Hodgkin's lymphoma;
the agonist is an OX40 agonist optionally selected from one or more of an antibody or antigen-binding fragment or small molecule or ligand that specifically binds thereto, OX86, Fc-OX40L, and GSK3174998;
the agonist is a GITR agonist optionally selected from one or more of an antibody or antigen-binding fragment or small molecule or ligand that specifically binds thereto, INCAGN01876, DTA-1, and MED11873;
the agonist is a CD137 agonist optionally selected from one or more of an antibody or antigen-binding fragment or small molecule or ligand that specifically binds thereto, utomilumab, and 4-1BB ligand;
the agonist is a CD27 agonist optionally selected from one or more of an antibody or antigen-binding fragment or small molecule or ligand that specifically binds thereto, varlilumab, and CDX-1127 (1F5);
the agonist is a CD28 agonist optionally selected from one or more of an antibody or antigen-binding fragment or small molecule or ligand that specifically binds thereto, and TAB08; and/or
the agonist is an HVEM agonist optionally selected from one or more of an antibody or antigen-binding fragment or small molecule or ligand that specifically binds thereto.

46. The method of claim 39, wherein the cancer vaccine is selected from one or more of Oncophage, a human papillomavirus HPV vaccine optionally Gardasil or Cervarix, a hepatitis B vaccine optionally Engerix-B, Recombivax HB, or Twinrix, and sipuleucel-T (Provenge), or comprises a cancer antigen selected from one or more of human Her2/neu, Her1/EGF receptor (EGFR), Her3, A33 antigen, B7H3, CD5, CD19, CD20, CD22, CD23 (IgE Receptor), MAGE-3, C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin, optionally wherein the subject has or is at risk for having a cancer that comprises the corresponding cancer antigen.

47. The method of claim 39, wherein the oncolytic virus selected from one or more of talimogene laherparepvec (T-VEC), coxsackievirus A21 (CAVATAK™), Oncorine (H101), pelareorep (REOLYSIN®), Seneca Valley virus (NTX-010), Senecavirus SVV-001, ColoAd1, SEPREHVIR (HSV-1716), CGTG-102 (Ad5/3-D24-GMCSF), GL-ONC1, MV-NIS, and DNX-2401.

48. The method of claim 39, wherein the cytokine selected from one or more of interferon (IFN)-α, IL-2, IL-12, IL-7, IL-21, and Granulocyte-macrophage colony-stimulating factor (GM-CSF).

49. The method of claim 39, wherein the cell-based immunotherapy agent comprises cancer antigen-specific T-cells, optionally ex vivo-derived T-cells.

50. The method of claim 49, wherein the cancer antigen-specific T-cells are selected from one or more of chimeric antigen receptor (CAR)-modified T-cells, and T-cell Receptor (TCR)-modified T-cells, tumor infiltrating lymphocytes (TILs), and peptide-induced T-cells.

51. The method of any one of claims 35-50, wherein the anti-VISTA antibody, or antigen-binding fragment thereof, and the at least one cancer immunotherapy agent are administered separately, as separate compositions.

52. The method of any one of claims 35-50, wherein the anti-VISTA antibody, or antigen-binding fragment thereof, and the at least one cancer immunotherapy agent are administered together as part of the same composition.

53. A method for treating a patient having an infectious disease, comprising administering to the patient a composition of claim 34, thereby treating the infectious disease.

54. The method of claim 53, wherein the infectious disease is a viral, bacterial, fungal optionally yeast, or protozoal infection.

55. A method of treating cancer in a patient in need thereof, comprising:
(a) incubating ex vivo-derived, autologous immune cells obtained from the patient with an anti-VISTA antibody, or antigen-binding fragment thereof, according to any one of claims 1-30; and
(b) administering the autologous immune cells to the patient.

56. The method of claim 55, wherein the autologous immune cells comprise lymphocytes, natural killer (NK) cells, macrophages, and/or dendritic cells.

57. The method of claim 56, wherein the lymphocytes comprise T-cells, optionally cytotoxic T-lymphocytes (CTLs).

58. The method of claim 57, wherein the T-cells comprise cancer antigen-specific T-cells.

59. The method of claim 58, wherein the cancer antigen-specific T-cells are selected from one or more of chimeric antigen receptor (CAR)-modified T-cells, T-cell Receptor (TCR)-modified T-cells, tumor infiltrating lymphocytes (TILs), and peptide-induced T-cells.
